SEQUENCE LISTING (49 sequences)

SEQ ID NO: 1 - protein, 25 aa, Artificial Sequence
  Signal peptide
MAVMAPRTLVLLLSGALALTQTWAG

SEQ ID NO: 2 - protein, 9 aa, Artificial Sequence
  Fusion peptide
ESSCDVKLV

SEQ ID NO: 3 - protein, 110 aa, Artificial Sequence
  CH2 domain, human IgG1 (allele *01)
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 4 - protein, 109 aa, Artificial Sequence
  CH2 domain, human IgG2 (allele *01)
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTK

SEQ ID NO: 5 - protein, 109 aa, Artificial Sequence
  CH2 domain, human IgG2 (allele *02)
APPVAGPSVFLFPPKPKDTLMISRTPEVTCVVVDVSHEDPEVQFNWYVDGMEVHNAKTKPREEQFNSTFRVVSVLTVVHQDWLNGKEYKCKVSNKGLPAPIEKTISKTK

SEQ ID NO: 6 - protein, 110 aa, Artificial Sequence
  CH2 domain, human IgG3 (allele *01)
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTK

SEQ ID NO: 7 - protein, 110 aa, Artificial Sequence
  CH2 domain, human IgG3 (allele *16)
APELLGGPSVFLFPPKPKDTLMISRTPEVTCVVVDVSHEDPEVQFKWYVDGVEVHNAKTKLREEQYNSTFRVVSVLTVLHQDWLNGKEYKCKVSNKALPAPIEKTISKAK

SEQ ID NO: 8 - protein, 110 aa, Artificial Sequence
  CH2 domain, human IgG3 (allele *09)
APELLGGPSVFLFPPKPKDTLMISRTPEVTCVVVDVSHEDPEVQFKWYVDGVEVHNAKTKPREEQYNSTFRVVSVLTVVHQDWLNGKEYKCKVSNKALPAPIEKTISKTK

SEQ ID NO: 9 - protein, 110 aa, Artificial Sequence
  CH2 domain, human IgG3 (allele *11)
APELLGGPSVFLFPPKPKDTLMISRTPEVTCVVVDVSHEDPEVQFKWYVDGVEVHNAKTKPREEQFNSTFRVVSVLTVLHQDWLNGKEYKCKVSNKALPAPIEKTISKTK

SEQ ID NO: 10 - protein, 110 aa, Artificial Sequence
  CH2 domain, human IgG3 (allele *14)
APELLGGPSVFLFPPKPKDTLMISRTPEVTCVVVDVSHEDPEVQFKWYVDGVEVHNAKTKLREEQYNSTFRVVSVLTVLHQDWLNGKEYKCKVSNKALPAPIEKTISKTK

SEQ ID NO: 11 - protein, 110 aa, Artificial Sequence
  CH2 domain, human IgG3 (allele *18)
APELLGGPSVFLFPPKPKDTLMISRTPEVTCVVVDVSHEDPEVQFKWYVDGVEVHNAKTKPWEEQYNSTFRVVSVLTVLHQDWLNGKEYKCKVSNKALPAPIEKTISKTK

SEQ ID NO: 12 - protein, 110 aa, Artificial Sequence
  CH2 domain, human IgG4 (allele *01)
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 13 - protein, 110 aa, Artificial Sequence
  CH2 domain, human IgG4 (allele *02)
APEFLGGPSVFLFPPKPKDTLMISRTPEVTCVVVDVSQEDPEVQFNWYVDGVEVHNAKTKPREEQFNSTYRVVSVLTVVHQDWLNGKEYKCKVSNKGLPSSIEKTISKAK

SEQ ID NO: 14 - protein, 106 aa, Artificial Sequence [sequence omitted]

SEQ ID NO: 15 - protein, 106 aa, Artificial Sequence
  CH3 domain, human IgG1 (allele *04)
GQPREPQVYTLPPSRDELTKNQVSLTCLVKGFYPSDIAVEWESNGQPENNYKTTPPVLDSDGSFFLYSKLTVDKSRWQQGNIFSCSVMHEALHNHYTQKSLSLSPG

SEQ ID NO: 16 - protein, 106 aa, Artificial Sequence
  CH3 domain, human IgG1 (allele *03)
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 17 - protein, 106 aa, Artificial Sequence
  CH3 domain, human IgG2 (allele *01)
GQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 18 - protein, 106 aa, Artificial Sequence
  CH3 domain, human IgG2 (allele *06)
GQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPG

SEQ ID NO: 19 - protein, 106 aa, Artificial Sequence
  CH3 domain, human IgG3 (allele *15)
GQPREPQVYTLPPSREEMTKNQVSLTCLVKGFYPSDIAVEWESNGQPENNYKTTPPMLDSDGSFFLYSKLTVDKSRWQQGNIFSCSVMHEALHNRYTQKSLSLSPG

SEQ ID NO: 20 - protein, 106 aa, Artificial Sequence
  CH3 domain, human IgG3 (allele *17)
GQPREPQVYTLPPSREEMTKNQVSLTCLVKGFYPSDIAMEWESSGQPENNYKTTPPVLDSDGSFFLYSKLTVDKSRWQQGNIFSCSVMHEALHNHYTQKSLSLSPG

SEQ ID NO: 21 - protein, 106 aa, Artificial Sequence
  CH3 domain, human IgG3 (allele *14)
GQPREPQVYTLPPSREEMTKNQVSLTCLVKGFYPSDIAVEWESNGQPENNYNTTPPMLDSDGSFFLYSKLTVDKSRWQQGNIFSCSVMHEALHNRYTQKSLSLSPG

SEQ ID NO: 22 - protein, 106 aa, Artificial Sequence
  CH3 domain, human IgG3 (allele *06)
GQPREPQVYTLPPSREEMTKNQVSLTCLVKGFYPSDIAVEWESSGQPENNYKTTPPMLDSDGSFFLYSKLTVDKSRWQQGNIFSCSVMHEALHNRFTQKSLSLSPG

SEQ ID NO: 23 - protein, 106 aa, Artificial Sequence
  CH3 domain, human IgG3 (allele *08)
GQPREPQVYTLPPSREEMTKNQVSLTCLVKGFYPSDIAVEWESNGQPENNYNTTPPMLDSDGSFFLYSKLTVDKSRWQQGNIFSCSVMHEALHNRFTQKSLSLSPG

SEQ ID NO: 24 - protein, 106 aa, Artificial Sequence
  CH3 domain, human IgG3 (allele *01)
GQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPG

SEQ ID NO: 25 - protein, 106 aa, Artificial Sequence
  CH3 domain, human IgG3 (allele *03)
GQPREPQVYTLPPSREEMTKNQVSLTCLVKGFYPSDIAVEWESSGQPENNYNTTPPVLDSDGSFFLYSRLTVDKSRWQEGNVFSCSVMHEALHNRFTQKSLSLSPG

SEQ ID NO: 26 - protein, 106 aa, Artificial Sequence
  CH3 domain, human IgG3 (allele *13)
GQPREPQVYTLPPSREEMTKNQVSLTCLVKGFYPSDIAVEWESSGQPENNYKTTPPMLDSDGSFFLYSKLTVDKSRWQEGNIFSCSVMHEALHNRFTQKSLSLSPG

SEQ ID NO: 27 - protein, 106 aa, Artificial Sequence
  CH3 domain, human IgG4 (allele *03)
GQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 28 - protein, 106 aa, Artificial Sequence
  CH3 domain, human IgG4 (allele *01)
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 29 - protein, 107 aa, Artificial Sequence [sequence omitted]

SEQ ID NO: 30 - protein, 107 aa, Artificial Sequence
  CL domain, human kappa light chain (allele *04)
RTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 31 - protein, 107 aa, Artificial Sequence
  CL domain, human kappa light chain (allele *05)
RTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 32 - protein, 107 aa, Artificial Sequence
  CL domain, human kappa light chain (allele *02)
RTVAAPSVFIFPPSDEQLKSGTASVVCLLNNFYPREAKVQWKVDNALQSGNSQESVTEQESKDSTYSLSSTLTLSKADYEKHKVYAGEVTHQGLSSPVTKSFNRGEC

SEQ ID NO: 33 - protein, 107 aa, Artificial Sequence
  CL domain, human kappa light chain (allele *03)
RTVAAPSVFIFPPSDEQLKSGTASVVCLLNNFYPREAKVQRKVDNALQSGNSQESVTEQESKDSTYSLSSTLTLSKADYEKHKVYACEVTHQGLSSPVTKSFNRGEC

SEQ ID NO: 34 - protein, 106 aa, Artificial Sequence
  CL domain, human lambda light chain (allele 3*02)
GQPKAAPSVTLFPPSSEELQANKATLVCLISDFYPGPVTVAWKADSSPVKAGVETTTPSKQSNNKYAASSYLSLTPEQWKSHKSYSCQVTHEGSTVEKTVAPTECS

SEQ ID NO: 35 - protein, 106 aa, Artificial Sequence
  CL domain, human lambda light chain (allele 3*03)
GQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 36 - protein, 106 aa, Artificial Sequence
  CL domain, human lambda light chain (allele 6*01)
GQPKAAPSVTLFPPSSEELQANKATLVCLISDFYPGAVKVAWKADGSPVNTGVETTTPSKQSNNKYAASSYLSLTPEQWKSHRSYSCQVTHEGSTVEKTVAPAECS

SEQ ID NO: 37 - protein, 106 aa, Artificial Sequence
  CL domain, human lambda light chain (allele 2*01)
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 38 - protein, 106 aa, Artificial Sequence
  CL domain, human lambda light chain (allele 7*01)
GQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 39 - protein, 106 aa, Artificial Sequence
  CL domain, human lambda light chain (allele 7*03)
GQPKAAPSVTLFPPSSEELQANKATLVCLVSDFNPGAVTVAWKADGSPVKVGVETTKPSKQSNNKYAASSYLSLTPEQWKSHRSYSCRVTHEGSTVEKTVAPAECS

SEQ ID NO: 40 - protein, 106 aa, Artificial Sequence
  CL domain, human lambda light chain (allele 1*02)
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 41 - protein, 98 aa, Artificial Sequence
  CH1 domain, human IgG1 (allele *01)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 42 - protein, 98 aa, Artificial Sequence
  CH1 domain, human IgG1 (allele *03)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 43 - protein, 98 aa, Artificial Sequence
  CH1 domain, human IgG2 (allele *04)
ASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKTV

SEQ ID NO: 44 - protein, 98 aa, Artificial Sequence
  CH1 domain, human IgG2 (allele *01)
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 45 - protein, 98 aa, Artificial Sequence
  CH1 domain, human IgG2 (allele *02)
ASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTV

SEQ ID NO: 46 - protein, 98 aa, Artificial Sequence
  CH1 domain, human IgG3 (allele *01)
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRV

SEQ ID NO: 47 - protein, 98 aa, Artificial Sequence
  CH1 domain, human IgG3 (allele *18)
ASTKGPSVFPLAPCSRSTSGGTAALGCLVKDYFPEPVTVSWNSGALTSGVHTFPAVLQYSGLYSLSSVVTVPSSSLGTQTYTCNVNHKPSNTKVDKRV

SEQ ID NO: 48 - protein, 98 aa, Artificial Sequence
  CH1 domain, human IgG3 (allele *17)
ASTKGPSVFPLAPCSRSTSGGTAALGCLVKDYFPEPVTVSWNSGALTSGVHTFPAVLQSSGLYSLSSVVTVPSSNFGTQTYTCNVNHKPSNTKVDKRV

SEQ ID NO: 49 - protein, 98 aa, Artificial Sequence
  CH1 domain, human IgG4 (allele *01)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

(19) United States
(12) Patent Application Publication
(10) Pub. No.: US 2026/0042847 A1
(43) Pub. Date: Feb. 12, 2026

(54) CYSTEINE ENGINEERED ANTIBODY CONSTRUCTS, CONJUGATES AND METHODS OF USE

(71) Applicant: ZYMEWORKS INC., Vancouver (CA)

(72) Inventors: James R. Rich, Vancouver (CA); Marlo Sanches, Vancouver (CA); Samir Das, Vancouver (CA); Patrick Farber, Vancouver (CA)

(21) Appl. No.: 18/551,851

(22) PCT Filed: Mar. 25, 2022
(86) PCT No.: PCT/CA2022/050453

§ 371 Date: Sep. 21, 2023

Related U.S. Application Data
(60) Provisional application No. 63/166,450, filed on Mar. 26, 2021.

(51) Int. Cl. C07K 16/28; A61K 47/68

(52) U.S. Cl. CPC C07K 16/2863; A61K 47/6803; A61K 47/68031; A61K 47/68037; C07K 16/28; C07K 2317/52; C07K 2317/526; C07K 2317/55; C07K 2317/73; C07K 2317/92

(57) ABSTRACT

Antibody constructs engineered to introduce at least one cysteine insertion mutation (“cysteine engineered antibody constructs”) are described. The inserted cysteine residue(s) may be used as a site for conjugation of one or more active agents to the antibody construct to provide conjugates, such as antibody-drug conjugates.

### FIELD

The present disclosure relates to the field of antibodies and, in particular, to antibodies engineered to include one or more cysteine insertion mutations and to conjugates comprising these antibodies and an active agent.

### BACKGROUND

Antibody drug conjugates (ADCs) represent a relatively new and promising class of therapeutics. ADCs are generally composed of a monoclonal antibody linked to a small molecule therapeutic (“drug”) via a linker. The drug-to-antibody ratio (DAR) and the specific sites of drug conjugation can influence the stability and exposure of the ADC (Hamblett, et al., 2004, Clin. Cancer Res., 10 (20): 7063-7070; Shen, et al., 2012, Nature Biotechnology, 30:184-189). Traditional methods for generation of ADCs through conjugation via native cysteine or lysine residues typically results in heterogeneous mixtures of conjugates, both in terms of drug load and conjugation, leading to possible liabilities in one or more of antibody stability, specificity, in vivo distribution, pharmacokinetics and/or efficacy.

Replacement of native amino acids in an antibody with cysteine residues for the purposes of site-specific conjugation was first described by Lyons in the context of labelling antibodies (Lyons, et al., 1990, Protein Eng Des Sel., 3 (8): 703-708). Site-specific drug conjugation at engineered cysteine residues in antibodies to solve the heterogeneity issue was later described (THIOMAB™) (Junutula, et al., 2008, Nat Biotechnol., 26:925-932; Vollmar, et al., 2017, Bioconjug Chem, 28 (10): 2538-2548; Ohri, et al., 2018, Bioconjug Chem., 29 (2): 473-485). Other examples of site-directed mutagenic incorporation of cysteine residues have also been described (for example, Sussman, et al., 2018, Protein Eng Des Sel., 31 (2): 47-54).

A related strategy involving insertion of a cysteine residue rather than substitution of a native amino acid with a cysteine residue has also been reported (Dimasi, et al., 2017, Mol. Pharmaceutics, 14 (5): 1501-1516; U.S. Patent Application Publication No. US 2018/0169255). Insertion of a cysteine residue after the site of one of the previously reported THIOMAB™ cysteine substitution positions (S239C), referred to as C239i, was used in MEDI2228, a clinical stage ADC. The C239i insertion results in abolition of FcγR binding and antibody-dependent cellular cytotoxicity (ADCC). A structural basis for these changes in function has been described (Gallagher, et al., 2019, Pharmaceutics, 11:546). Other cysteine insertion approaches have also been described (U.S. Patent Application Publication No. US 2020/0129635 and International Patent Application Publication No. WO 2018/233572).

This background information is provided for the purpose of making known information believed by the applicant to be of possible relevance to the present disclosure. No admission is necessarily intended, nor should be construed, that any of the preceding information constitutes prior art against the claimed invention.

### SUMMARY

Described herein are cysteine engineered antibody constructs, conjugates and methods of use.

In one aspect, the present disclosure relates to a cysteine engineered antibody construct comprising a VH domain, a VH domain and a VL domain, an Fc region, or a combination thereof, the Fc region comprising a CH2 domain and/or a CH3 domain, the antibody construct comprising one or more cysteine insertion mutations selected from: (a) an insertion of a cysteine residue between positions 39 and 40 in the VL domain; (b) an insertion of a cysteine residue between positions 40 and 41 in the VL domain; (c) an insertion of a cysteine residue between positions 126 and 127 in the CL domain; (d) an insertion of a cysteine residue between positions 148 and 149 in the CL domain; (e) an insertion of a cysteine residue between positions 149 and 150 in the CL domain; (f) an insertion of a cysteine residue between positions 9 and 10 in the VH domain; (g) an insertion of a cysteine residue between positions 169 and 170 in the CH1 domain; (h) an insertion of a cysteine residue between positions 237 and 238 in the CH2 domain; (i) an insertion of a cysteine residue between positions 295 and 296 in the CH2 domain, and (j) an insertion of a cysteine residue between positions 299 and 300 in the CH2 domain, wherein the numbering of amino acids in the VL, CL, VH and CH1 domains is Kabat numbering and the numbering of amino acids in the CH2 domain is EU numbering, and wherein antibody construct is based on an immunoglobulin G (IgG).

In another aspect, the present disclosure relates to a conjugate comprising the cysteine engineered antibody construct as described in any one of the embodiments disclosed herein, and one or more active agents conjugated to each of the one or more inserted cysteine residues.

In another aspect, the present disclosure relates to a conjugate having Formula (I):

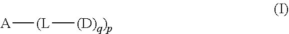

wherein A is a cysteine engineered antibody construct; L is a linker; D is an active agent; q is an integer between 1 and 4, and p is an integer between 1 and 8, wherein the cysteine engineered antibody construct comprises a VH domain, a VH domain and a VL domain, an Fc region, or a combination thereof, the Fc region comprising a CH2 domain and/or a CH3 domain, and wherein the cysteine engineered antibody construct comprises one or more cysteine insertion mutations selected from: (a) an insertion of a cysteine residue between positions 39 and 40 in the VL domain; (b) an insertion of a cysteine residue between positions 40 and 41 in the VL domain; (c) an insertion of a cysteine residue between positions 126 and 127 in the CL domain; (d) an insertion of a cysteine residue between positions 148 and 149 in the CL domain; (e) an insertion of a cysteine residue between positions 149 and 150 in the CL domain; (f) an insertion of a cysteine residue between positions 9 and 10 in the VH domain; (g) an insertion of a cysteine residue between positions 169 and 170 in the CH1 domain; (h) an insertion of a cysteine residue between positions 237 and 238 in the CH2 domain; (i) an insertion of a cysteine residue between positions 295 and 296 in the CH2 domain, and (j) an insertion of a cysteine residue between positions 299 and 300 in the CH2 domain, wherein the numbering of amino acids in the VL, CL, VH and CH1 domains is Kabat numbering and the numbering of amino acids in the CH2 domain is EU numbering, wherein the cysteine engineered antibody construct is based on an immunoglobulin G (IgG), and wherein each D is linked to an inserted cysteine residue via L.

In another aspect, the present disclosure relates to a composition comprising a conjugate as described in any one of the embodiments disclosed herein, and a pharmaceutically acceptable carrier or diluent.

In another aspect, the present disclosure relates to a method of treating a disease or disorder in a subject in need thereof comprising administering an effective amount of a conjugate as described herein, where the active agent comprised by the conjugate is a therapeutic agent.

In another aspect, the present disclosure relates to a conjugate as described herein for use in therapy, where the active agent comprised by the conjugate is a therapeutic agent.

In another aspect, the present disclosure relates to a use of a conjugate as described in herein in the manufacture of a medicament for the treatment of a subject in need thereof, where the active agent comprised by the conjugate is a therapeutic agent.

In another aspect, the present disclosure relates to a method of preparing a conjugate as described in any one of the embodiments disclosed herein comprising submitting the cysteine engineered antibody construct to reducing conditions such that the thiol group of the one or more inserted cysteine residues is reduced, and reacting a thiol reactive linker-active agent with the antibody construct under conditions that permit formation of a bond between the linker and the reduced thiol.

In another aspect, the present disclosure relates to a method of preparing an antibody-drug conjugate having a pre-determined drug-to-antibody ratio (DAR), the method comprising: (i) providing a cysteine engineered antibody construct comprising a VH domain, a VH domain and a VL domain, an Fc region, or a combination thereof, the Fc region comprising a CH2 domain and/or a CH3 domain, and the antibody construct comprising one or more cysteine insertion mutations selected from: (a) an insertion of a cysteine residue between positions 39 and 40 in the VL domain; (b) an insertion of a cysteine residue between positions 40 and 41 in the VL domain; (c) an insertion of a cysteine residue between positions 126 and 127 in the CL domain; (d) an insertion of a cysteine residue between positions 148 and 149 in the CL domain; (e) an insertion of a cysteine residue between positions 149 and 150 in the CL domain; (f) an insertion of a cysteine residue between positions 9 and 10 in the VH domain; (g) an insertion of a cysteine residue between positions 169 and 170 in the CH1 domain; (h) an insertion of a cysteine residue between positions 237 and 238 in the CH2 domain; (i) an insertion of a cysteine residue between positions 295 and 296 in the CH2 domain, and (j) an insertion of a cysteine residue between positions 299 and 300 in the CH2 domain, and (ii) reacting the cysteine engineered antibody construct with a drug-linker to provide the antibody-drug conjugate; wherein the pre-determined DAR is 1, 2, 3, 4, 5, 6, 7 or 8, and the cysteine engineered antibody construct comprises the same number of cysteine insertion mutations as the pre-determined DAR, wherein the numbering of amino acids in the VL, CL, VH and CHI domains is Kabat numbering and the numbering of amino acids in the CH2 domain is EU numbering, and wherein the cysteine engineered antibody construct is based on an immunoglobulin G (IgG).

In another aspect, the present disclosure relates to a polynucleotide or set of polynucleotides encoding a cysteine engineered antibody construct as described in any one of the embodiments disclosed herein.

In another aspect, the present disclosure relates to a vector comprising one or more polynucleotides encoding a cysteine engineered antibody construct as described in any one of the embodiments disclosed herein.

In another aspect, the present disclosure relates to a host cell comprising a vector comprising one or more polynucleotides encoding a cysteine engineered antibody construct as described in any one of the embodiments disclosed herein.

### BRIEF DESCRIPTION OF THE DRAWINGS

FIG. 1 shows the structures of the drug linkers: (A) MCvcPAB-Tubulysin M, (B) MCvcPABC-MMAE, and (C) MTvcCompound 1.

FIG. 2 shows (A) non-reducing capillary-electrophoresis SDS (CE-SDS) gel analysis, and (B) reducing CE-SDS gel analysis, of 30 exemplary antibody-drug conjugates (ADCs) prepared using cysteine insertion variants, as compared to unconjugated control (v17427) and control ADCs. Lane A: Unconjugated control (v17427); Lanes B-D. variant v22760 (L_K39.5C) conjugated to MCvcPABC-MMAE (B), MCvcPAB-Tubulysin M (C) and MTvcCompound 1 (D); Lanes E-G: variant v22761 (L_K126.5C) conjugated to MCvcPABC-MMAE (E), MCvcPAB-Tubulysin M (F) and MTvcCompound 1 (G); Lanes H-J: variant v22765 (H_G237.5C) conjugated to MCvcPABC-MMAE (H), MCvcPAB-Tubulysin M (I) and MTvcCompound 1 (J); Lanes K-M: variant v22768 (H_Q295.5C) conjugated to MCvcPABC-MMAE (K), MCvcPAB-Tubulysin M (L) and MTvcCompound 1 (M); Lanes N-P: variant v27321 (L_W148.5C) conjugated to MCvcPABC-MMAE (N), MCvcPAB-Tubulysin M (O) and MTvcCompound 1 (P); Lanes Q-S: variant v27322 (L_K149.5C) conjugated to MCvcPABC-MMAE (Q), MCvcPAB-Tubulysin M (R) and MTvcCompound 1 (S); Lanes T-V: variant v28983 (L_P40.5C) conjugated to MCvcPABC-MMAE (T), MCvcPAB-Tubulysin M (U) and MTvcCompound 1 (V); Lanes W-Y: variant v28989 (H_A9.5C) conjugated to MCvcPABC-MMAE (W), MCvcPAB-Tubulysin M (X) and MTvcCompound 1 (Y); Lanes Z-BB: variant v28993 (H_G169.5C) conjugated to MCvcPABC-MMAE (Z), MCvcPAB-Tubulysin M (AA) and MTvcCompound 1 (BB); Lanes CC-EE: variant v29001 (H_T299.5C) conjugated to MCvcPABC-MMAE (CC), MCvcPAB-Tubulysin M (DD) and MTvcCompound 1 (EE); Lanes FF-HH: variant v22758 (H_A114C; control) conjugated to MCvcPABC-MMAE (FF), MCvcPAB-Tubulysin M (GG) and MTvcCompound 1 (HH); Lanes II-KK: variant v29013 (H_S239.5C; control) conjugated to MCvcPABC-MMAE (II), MCvcPAB-Tubulysin M (JJ) and MTvcCompound 1 (KK). MW markers (from top to bottom): 119, 68, 48, 29, 21, 16 kDa.

FIG. 3 shows the results of immunoprecipitation mass spectrometry (IPMS)-mediated DAR analysis of antibody-drug conjugates (ADCs) comprising cysteine insertion variants conjugated to the drug-linker MTvcCompound 1 after incubation with mouse plasma. Both remaining DAR (closed circles; left-hand axis) and % maleimide ring-opening (open circles; right-hand axis) are shown. (A) variant v22760 (L_K39.5C), (B) variant v22761 (L_K126.5C), (C) variant v22765 (H_G237.5C), (D) variant v22768 (H_Q295.5C), (E) variant v27321 (L_W148.5C), (F) variant v27322 (L_K149.5C), (G) variant v28983 (L_P40.5C), (H) variant v28989 (H_A9.5C), (I) variant v28993 (H_G169.5C), (J) variant v29001 (H_T299.5C), (K) variant v22758 (H_A114C; control), and (L) variant v29013 (H_S239.5C; control).

FIG. 4 shows the results of immunoprecipitation mass spectrometry (IPMS)-mediated DAR analysis of antibody-drug conjugates (ADCs) comprising cysteine insertion variants conjugated to the drug-linker MCvcPABC-MMAE after incubation with mouse plasma. Both remaining DAR (closed circles; left-hand axis) and % maleimide ring-opening (open circles; right-hand axis) are shown. (A) variant v22760 (L_K39.5C), (B) variant v22761 (L_K126.5C), (C) variant v22765 (H_G237.5C), (D) variant v22768 (H_Q295.5C), (E) variant v27321 (L_W148.5C), (F) variant v27322 (L_K149.5C), (G) variant v28983 (L_P40.5C), (H) variant v28989 (H_A9.5C), (I) variant v28993 (H_G169.5C), (J) variant v29001 (H_T299.5C), (K) variant v22758 (H_A114C; control), and (L) variant v29013 (H_S239.5C; control).

FIG. 5 shows the results of immunoprecipitation mass spectrometry (IPMS)-mediated DAR analysis of antibody-drug conjugates (ADCs) comprising cysteine insertion variants conjugated to the drug-linker MCvcPAB-Tubulysin M after incubation with mouse plasma. Remaining DAR (closed circles, solid line; left-hand axis), % maleimide ring-opening (open circles; right-hand axis) and % decomposition (acetyl loss) of MCvcPAB-Tubulysin M (closed circles, dashed line) are shown. (A) variant v22760 (L_K39.5C), (B) variant v22761 (L_K126.5C), (C) variant v22765 (H_G237.5C), (D) variant v22768 (H_Q295.5C), (E) variant v27321 (L_W148.5C), (F) variant v27322 (L_K149.5C), (G) variant v28983 (L_P40.5C), (H) variant v28989 (H_A9.5C), (I) variant v28993 (H_G169.5C), (J) variant v29001 (H_T299.5C), (K) variant v22758 (H_A114C; control), and (L) variant v29013 (H_S239.5C; control).

FIG. 6 shows the results of in vitro cytotoxicity testing of ADCs comprising cysteine insertion variants conjugated to MTvcCompound 1 at DAR 1, 2 or 3 on different c-Met expressing cell lines, (A) EBC-1 cell line (high c-Met-expressing), and (B) HT-29 cell line (mid c-Met-expressing), compared to control ADC (v17427-MTvcCompound 1, DAR 4).

FIG. 7 shows the in vivo anti-tumor activity of ADCs comprising cysteine insertion variants conjugated to MTvcCompound 1 at DAR 1, 2 or 3 in mid c-Met expressing colorectal cancer xenograft model HT-29 compared to DAR4 controls at toxin-matched doses of (A) 6 mg/kg (DAR1), 3 mg/kg (DAR2), 2 mg/kg (DAR3) and 1.5 mg/kg (DAR4), and (B) 12 mg/kg (DAR1), 6 mg/kg (DAR2), 4 mg/kg (DAR3) and 3 mg/kg (DAR4).

FIG. 8 shows the in vivo anti-tumor activity of ADCs comprising cysteine insertion variants conjugated to MTvcCompound 1 at DAR 1, 2 or 3 in high c-Met expressing non-small cell lung cancer xenograft model H1975 compared to DAR4 controls at toxin-matched doses of (A) 4 mg/kg (DAR1), 2 mg/kg (DAR2), 1.3 mg/kg (DAR3) and 1 mg/kg (DAR4), and (B) 24 mg/kg (DAR1), 12 mg/kg (DAR2), 8 mg/kg (DAR3) and 6 mg/kg (DAR4).

FIG. 9 presents a sequence alignment of the CH1 domains of human IgG1 (allele *01 [SEQ ID NO:41] and allele *03 [SEQ ID NO:42]), IgG3 (allele *01 [SEQ ID NO:43], allele *18 [SEQ ID NO:44] and allele *17 [SEQ ID NO:45]), IgG2 (allele *04 [SEQ ID NO:46]), IgG4 allele *01 [SEQ ID NO:47], IgG2 allele *01 [SEQ ID NO:48]) and IgG2 (allele *02 [SEQ ID NO:49]).

FIG. 10 presents a sequence alignment of the CH2 domains of human IgG1 (allele *01 [SEQ ID NO:3]), IgG3 (allele *01 [SEQ ID NO:4], IgG3 (allele *16 [SEQ ID NO:4], allele *09 [SEQ ID NO:5], allele *09 [SEQ ID NO:6], allele *11 [SEQ ID NO:7], allele *14 [SEQ ID NO:8] and allele *18 [SEQ ID NO:9]), IgG4 (allele *01 [SEQ ID NO:10] and allele *02 [SEQ ID NO: 11]), and IgG2 (allele *01 [SEQ ID NO:12], and allele *02 [SEQ ID NO:13].

FIG. 11 presents a sequence alignment of the CH3 domains of human IgG1 (allele *01 [SEQ ID NO: 14], allele *04 [SEQ ID NO:15] and allele *03 [SEQ ID NO:16]), IgG2 (allele *01 [SEQ ID NO: 17] and allele *06 [SEQ ID NO: 18]), IgG3 (allele *15 [SEQ ID NO: 19], allele *17 [SEQ ID NO:20], human IgG4 (allele *03 [SEQ ID NO:21]), human IgG3 (allele *14 [SEQ ID NO: 22]), human IgG4 (allele *01 [SEQ ID NO:23]), human IgG3 (allele *06 [SEQ ID NO:24]), human IgG3 (allele *08 [SEQ ID NO:25]), human IgG3 (allele *01 [SEQ ID NO:26]), human IgG3 (allele *03 [SEQ ID NO:27]), human IgG3 (allele *13 [SEQ ID NO:28]),

FIG. 12 presents a sequence alignment of the CL domains of human kappa light chain (allele *01 [SEQ ID NO:29], allele *04 [SEQ ID NO:30], allele *05 [SEQ ID NO:31], allele *02 [SEQ ID NO:32] and allele *03 [SEQ ID NO:33]) and human lambda light chain (allele 3*02 [SEQ ID NO:34], allele 3*03 [SEQ ID NO:35], allele 6*01 [SEQ ID NO:36], allele 2*01 [SEQ ID NO: 37], allele 7*01 [SEQ ID NO:38], allele 7*03 [SEQ ID NO:39] and allele 1*02 [SEQ ID NO: 40]).

FIG. 13 shows hydrophobic interaction chromatography (HIC) profiles for (A) variant v29013 (H_S239.5C; control) conjugated to the drug-linker MCvcPABC-MMAE, and (B) variant v29001 (H_T299.5C) conjugated to the drug-linker MCvcPABC-MMAE.

FIG. 14 shows hydrophobic interaction chromatography (HIC) profiles for (A) variant v29013 (H_S239.5C; control) conjugated to the drug-linker MTvcCompound 1, and (B) variant v29001 (H_T299.5C) conjugated to the drug-linker MTvcCompound 1.

FIG. 15 shows differential scanning calorimetry (DSC) profiles for (A) variant v29013 (H_S239.5C; control), and (B) variant v29001 (H_T299.5C), each compared to a control variant comprising a cysteine substitution mutation (v27320, L_K149C).

FIG. 16 shows (A) the hydrophobic interaction chromatography (HIC) profile of the ADC v35074-MTvcCompound 1 (DAR 6) where two distinct peaks were observed; peak eluted at 7.07 mins represents DAR 5 and peak eluted at 7.5 mins represents DAR 6, and (B) the size exclusion chromatography (SEC) profile of the same ADC where fractions eluted at 3.3 mins represent monomer (99%).

FIG. 17 shows the reduced LC-MS profiles for the light chain (LC) (A), heavy chain 1 (B) and heavy chain 2 (C) of the ADC v35074-MTvcCompound 1 (DAR 6) after EndoS treatment.

FIG. 18 shows the capillary electrophoresis-SDS (CE-SDS) profile of the cysteine insertion variant v35074 as unconjugated antibody and conjugated to the drug-linker MTvcCompound 1. Lane 1: protein ladder; Lane 2: Trastuzumab control (non-reduced (NR)); Lane 3: unconjugated v35074 (NR); Lane 4: v35074-MTvcCompound 1 (NR); Lane 5: trastuzumab (reduced (R)); Lane 6: unconjugated v35074 (R); Lane 7: v35074-MTvcCompound 1 (R)

### DETAILED DESCRIPTION

The present disclosure relates to antibody constructs engineered to introduce at least one cysteine insertion mutation (“cysteine engineered antibody constructs”). A “cysteine insertion mutation” in this context refers to a non-native cysteine residue that is introduced between two amino acid residues present in the parental antibody construct sequence. The inserted cysteine residue(s) may be used as a site for conjugation of one or more active agents, such as therapeutic, diagnostic and labelling agents, to the antibody construct to provide conjugates.

Certain embodiments of the present disclosure relate to conjugates comprising a cysteine engineered antibody construct and an active agent covalently attached to an inserted cysteine residue in antibody construct. The conjugates may find use in various therapeutic and diagnostic applications.

### Definitions

Unless defined otherwise, all technical and scientific terms used herein have the same meaning as commonly understood by one of ordinary skill in art.

As used herein, the term “about” refers to an approximately +/−10% variation from a given value. It is to be understood that such a variation is always included in any given value provided herein, whether or not it is specifically referred to.

The use of the word “a” or “an” when used herein in conjunction with the term “comprising” may mean “one,” but it is also consistent with the meaning of “one or more,” “at least one” and “one or more than one.”

As used herein, the terms “comprising,” “having,” “including” and “containing,” and grammatical variations thereof, are inclusive or open-ended and do not exclude additional, unrecited elements and/or method steps. The term “consisting essentially of” when used herein in connection with a composition, use or method, denotes that additional elements and/or method steps may be present, but that these additions do not materially affect the manner in which the recited composition, method or use functions. The term “consisting of” when used herein in connection with a composition, use or method, excludes the presence of additional elements and/or method steps. A composition, use or method described herein as comprising certain elements and/or steps may also, in certain embodiments consist essentially of those elements and/or steps, and in other embodiments consist of those elements and/or steps, whether or not these embodiments are specifically referred to.

The term “antibody construct” as used herein, encompasses full-length antibodies and functional fragments of full-length antibodies. Functional antibody fragments include antigen-binding fragments (such as Fab′ fragments, F(ab′)₂ fragments, Fab fragments, single chain variable regions (scFv) and single domain antibodies (sdAbs)), as well as Fc fragments comprising an Fc region capable of binding to one or more Fc receptors (FcR). The term “antibody constructs” also encompasses Fc fusion proteins comprising an Fc region and one or more heterologous polypeptides.

A full-length antibody comprises a heavy chain and a light chain assembled as a hetero-tetramer containing two heavy chains and two light chains. The heavy chain typically comprises the domains (from N- to C-terminus): VH-CH1-hinge-CH2-CH3, and the light chain typically comprises the domains (from N- to C-terminus): VL-CL. Unless otherwise specified, numbering of amino acids in the VH, CHI, VL and CL domains used herein is Kabat numbering, and numbering of amino acid residues in the CH2 and CH3 domains and the hinge region used herein is EU numbering, also called the EU index (both numbering systems are described in Kabat et al, Sequences of Proteins of Immunological Interest, 5th Ed. Public Health Service, National Institutes of Health, Bethesda, MD (1991)).

The terms “Fc region” and “Fc,” as used interchangeably herein, refer to a C-terminal region of an immunoglobulin heavy chain. Although the boundaries of the Fc region of an immunoglobulin heavy chain may vary, the human IgG heavy chain Fc region sequence is usually defined as extending from position 239 (EU numbering) to the C-terminus of the heavy chain. An “Fc polypeptide” of a dimeric Fc refers to one of the two polypeptides forming the dimeric Fc domain, i.e. a polypeptide comprising C-terminal constant regions of an immunoglobulin heavy chain that is capable of stable self-association. An Fc region typically comprises a CH2 domain and a CH3 domain, but in some embodiments may comprise just a CH2 domain or just a CH3 domain. The Fc region may also be considered to encompass the hinge region in certain embodiments.

The “CH2 domain” of a human IgG Fc region is typically defined as extending from position 239 to position 340. The “CH3 domain” is typically defined as comprising the amino acids residues C-terminal to the CH2 domain in an Fc region, i.e. from position 341 to position 447. The “hinge region” of human IgGI is generally defined as extending from position 216 to position 238 (Burton, 1985, Molec. Immunol., 22:161-206). Hinge regions of other IgG isotypes may be aligned with the IgGI sequence by aligning the first and last cysteine residues that form inter-heavy chain disulfide bonds.

An “Fc fusion protein,” in the context of the present disclosure, is a protein comprising all or a part (for example, a CH2 domain or a CH3 domain) of a Fc region fused to a heterologous protein or polypeptide.

The terms “derived from” and “based on” when used herein to describe an amino acid sequence, mean that the subject amino acid sequence is substantially identical to the stated reference amino acid sequence.

The term “substantially identical” when herein in connection with an amino acid sequence means that, when optimally aligned (for example using the methods described below), the amino acid sequence shares at least 85%, at least 90%, at least 95%, at least 96%, at least 97%, at least 98%, or at least 99% sequence identity with its reference amino acid sequence. Percent identity between two amino acid sequences may be determined in various ways known in art, for example, using publicly available computer software such as Smith Waterman Alignment (Smith & Waterman, 1981, J Mol Biol 147:195-7); “BestFit” (Smith & Waterman, 1981, Advances in Applied Mathematics, 482-489); BLAST (Basic Local Alignment Search Tool; (Altschul, et al., 1990, J Mol Biol, 215:403-10) and variations and updates thereof; ALIGN, ALIGN-2, CLUSTAL or Megalign (DNASTAR) software. In addition, those skilled in art can determine appropriate parameters for measuring alignment, including algorithms needed to achieve maximal alignment over the length of the sequences being compared. In general, for peptides, the length of comparison sequences will be at least 10 amino acids, but one skilled in art will understand that the actual length will depend on the overall length of the sequences being compared. In certain embodiments, the length of comparison sequences may be the full-length of the protein or peptide sequence.

The term “isolated,” as used herein with reference to a material, means that the material is removed from its original environment (for example, the natural environment if it is naturally occurring). For example, a naturally occurring polynucleotide or polypeptide present in a living animal is not isolated, but the same polynucleotide or polypeptide separated from some or all of the co-existing materials in the natural system, is isolated. Such polynucleotides could be part of a vector and/or such polynucleotides or polypeptides could be part of a composition, and still be isolated in that such vector or composition is not part of its natural environment.

It is to be understood that the positive recitation of a feature in one embodiment described herein serves as a basis for excluding the feature in an alternative embodiment. In particular, where a list of options is presented for a given embodiment or claim, it is to be understood that one or more option may be deleted from the list and the shortened list may form an alternative embodiment, whether or not such an alternative embodiment is specifically referred to.

It is contemplated that any embodiment discussed herein can be implemented with respect to any method, use or composition disclosed herein, and vice versa.

### Cysteine Engineered Antibody Constructs

The cysteine engineered antibody constructs of the present disclosure are antibody constructs comprising one or more cysteine insertion mutations. The cysteine engineered antibody construct may be, for example, a full-length antibody, a functional fragment of a full-length antibody or an Fc fusion protein. Functional antibody fragments include, for example, antigen-binding fragments and Fc fragments. Examples of antigen-binding fragments include, but are not limited to, variable regions of light and/or heavy chains of an antibody (VL, VH), variable fragments (Fv), Fab′ fragments, F(ab′)₂ fragments, Fab fragments, single chain variable regions (scFv), complementarity determining regions (CDRs) and single domain antibodies (sdAbs). Fc fragments typically include the CH2 and CH3 domains of an antibody and are capable of binding one or more Fc receptors (FcR). Fc fragments may optionally comprise a hinge region.

Fc fusion proteins comprise an Fc region fused or covalently attached to one or more heterologous polypeptides. In certain embodiments, the Fc fusion proteins comprise an Fc region fused or covalently attached to one or more target binding domains. Examples of target binding domains that may be included in an Fc fusion protein in certain embodiments include, but are not limited to, receptors, receptor fragments (such as extracellular portions), ligands, cytokines and heterologous antigen-binding antibody fragments (such as an antigen-binding fragment from a different antibody class or subclass). The one or more heterologous polypeptides may be fused or covalently attached to the Fc region directly or via a linker, for example, an amino acid-based linker.

Certain embodiments of the present disclosure relate to cysteine engineered antibody constructs that comprise an antigen-binding domain, an Fc region, or both an antigen-binding domain and an Fc region. In some embodiments, the cysteine engineered antibody constructs comprise an antigen-binding domain that comprises a VH domain or a VH domain and a VL domain. In some embodiments, the cysteine engineered antibody constructs comprise an Fc region that comprises a CH2 domain and/or a CH3 domain. In some embodiments, the cysteine engineered antibody constructs comprise an Fc region that comprises a CH2 domain and a CH3 domain.

Certain embodiments of the present disclosure relate to cysteine engineered antibody constructs that are full-length antibodies. In such embodiments, the cysteine engineered antibody may be, for example, a monoclonal antibody, a human antibody, a chimeric antibody or a humanized antibody. In this context, the full-length antibody may comprise one or more than one Fab region. For example, the full-length antibody may be a one-armed (monovalent) antibody (OAA), a bivalent antibody or a multivalent antibody.

Certain embodiments relate to cysteine engineered antibody constructs that are functional antibody fragments. In some embodiments, the cysteine engineered antibody construct is a functional fragment comprising at least one antigen-binding domain, such as a Fab, an scFv or an sdAb. In some embodiments, the cysteine engineered antibody construct comprises more than one antigen-binding domain, where the antigen-binding domains may be, for example, Fabs, scFvs or a combination thereof. In some embodiments, the cysteine engineered antibody construct comprises two or more antigen-binding domains joined with a linker, such as in a tandem scFv format or an scFv-Fab format.

In some embodiments, the cysteine engineered antibody may be a bispecific or multispecific antibody comprising two or more antigen-binding domains, each binding to a different antigenic epitope.

Certain embodiments relate to cysteine engineered antibody constructs that are Fc fusion proteins.

When the cysteine engineered antibody constructs comprise one or more antigen-binding domains, each antigen-binding domain binds to a target antigen. Target antigens are typically cell surface molecules, such as proteins, lipids or polysaccharides, found on the surface of a target cell, such as a tumor cell, a virally infected cell, a bacterially infected cell, a damaged red blood cell, an arterial plaque cell, an inflamed tissue cell or a fibrotic tissue cell. Examples of target antigens include, but are not limited to, tumor-associated antigens (TAA), cell surface receptor proteins, transmembrane proteins, signalling proteins, cell survival regulatory factors, cell proliferation regulatory factors, molecules associated with tissue development or differentiation, lymphokines, cytokines, molecules involved in cell cycle regulation, molecules involved in vasculogenesis and molecules associated with angiogenesis. Certain embodiments relate to cysteine engineered antibody constructs comprising at least one antigen-binding domain that binds to a tumor-associated antigen (TAA).

The cysteine engineered antibody constructs of the present disclosure are derived from an immunoglobulin G (IgG). In certain embodiments, the cysteine engineered antibody construct is derived from a human IgG. In some embodiments, the cysteine engineered antibody construct is derived from a human IgG1, IgG2, IgG3 or IgG4. In some embodiments, the cysteine engineered antibody construct is derived from an IgG1. In some embodiments, the cysteine engineered antibody construct is derived from a human IgG1.

In certain embodiments in which the cysteine engineered antibody construct comprises a cysteine insertion in the light chain, the antibody construct may comprise a kappa light chain or a lambda light chain. In some embodiments, in which the cysteine engineered antibody construct comprises a cysteine insertion in the light chain, the antibody construct comprises a kappa light chain.

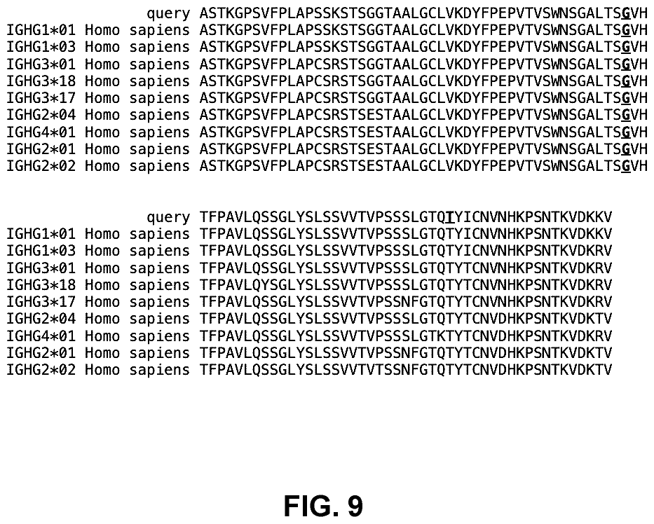

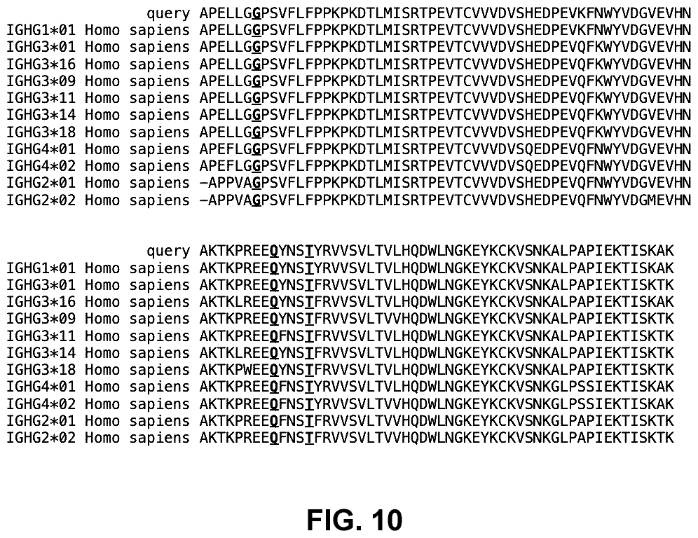

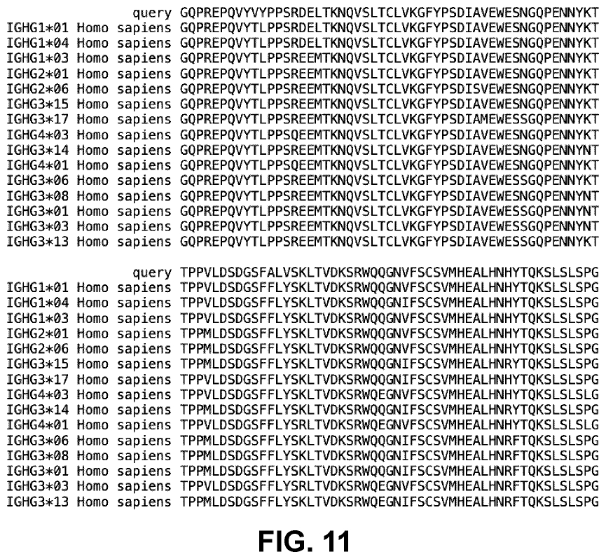

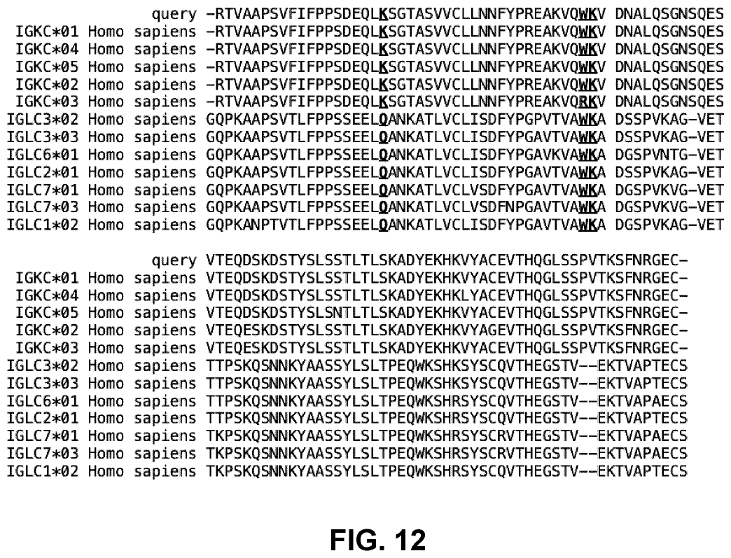

The amino acid sequences of the CH1, CH2 and CH3 domains for human IgG1, IgG2, IgG3 and IgG4, and of the kappa and lambda light chains are known in art (see, for example, the sequences provided on the International ImMunoGeneTics information system (IMGTR) website). Representative amino acid sequences of the CH1, CH2 and CH3 domains for various alleles of human IgG1, IgG2, IgG3 and IgG4 are also provided in FIGS. 9-11, respectively, and representative amino acid sequences for alleles of kappa and lambda CL domains are provided in FIG. 12.

In certain embodiments, the cysteine insertion mutation has no effect or a minimal effect on the stability of the cysteine engineered antibody construct as determined by melting temperature (Tm). By “no effect or minimal effect” it is meant that the Tm of the domain of the cysteine engineered antibody construct into which the cysteine residue is inserted is within (i.e. ±) 0° C. to 8° C. of the Tm of the same domain in the corresponding parental antibody construct (that lacks the cysteine insertion mutation). For example, for a cysteine engineered antibody construct comprising a cysteine residue inserted in the CH2 domain, the CH2 domain Tm of the cysteine engineered antibody construct is within 0° C. to 8° C. of the CH2 domain Tm of the corresponding parental antibody construct. In some embodiments, the Tm of the domain of the cysteine engineered antibody construct into which the cysteine residue is inserted is within 0° C. to 7° C. of the Tm of the same domain in the corresponding parental antibody construct. In some embodiments, the Tm of the domain of the cysteine engineered antibody construct into which the cysteine residue is inserted is within 0° C. to 6° C., or within 0° C. to 5° C., of the Tm of the same domain in the corresponding parental antibody construct.

The Tm of an antibody construct may be determined by various techniques known in the art, for example, circular dichroism (CD), differential scanning calorimetry (DSC) or differential scanning fluorimetry (DSF). In certain embodiments, the Tm difference between the cysteine engineered antibody construct and the corresponding parental antibody construct is determined by DSC.

In certain embodiments, the cysteine engineered antibody constructs of the present disclosure include the same cysteine insertion mutation in each chain of the antibody construct, for example in both heavy chains or in both light chains, resulting in an antibody construct that when conjugated to an active agent has an average drug-to-antibody ratio (DAR) of 2.

Certain embodiments of the present disclosure relate to “DAR-tuned” cysteine engineered antibody constructs. A “DAR-tuned” antibody construct in this context is a cysteine engineered antibody construct that comprises a cysteine insertion mutation in only one chain of the construct (allowing for DAR 1 conjugates) or that comprises a combination of cysteine insertion mutations (allowing for conjugates having DAR≥2, for example, DAR 3, DAR 4 or DAR 6).

### Cysteine Insertion Mutations

By combining structure-based computational approaches and experimental testing, appropriate sites for cysteine insertion mutations were identified in the IgG structure as described in the Examples herein. In certain embodiments, the cysteine engineered antibody constructs of the present disclosure comprise one or more cysteine insertion mutations selected from:
(a) an insertion of a cysteine residue between positions 39 and 40 in the VL domain;
(b) an insertion of a cysteine residue between positions 40 and 41 in the VL domain;
(c) an insertion of a cysteine residue between positions 126 and 127 in the CL domain;
(d) an insertion of a cysteine residue between positions 148 and 149 in the CL domain;
(e) an insertion of a cysteine residue between positions 149 and 150 in the CL domain;
(f) an insertion of a cysteine residue between positions 9 and 10 in the VH domain;
(g) an insertion of a cysteine residue between positions 169 and 170 in the CH1 domain;
(h) an insertion of a cysteine residue between positions 237 and 238 in the CH2 domain;
(i) an insertion of a cysteine residue between positions 295 and 296 in the CH2 domain, and
(j) an insertion of a cysteine residue between positions 299 and 300 in the CH2 domain.

It will be appreciated that the cysteine insertion mutation(s) that can be included in a given antibody construct will be dependent on the format of the antibody construct. A full-length antibody construct, for example, may comprise cysteine insertion mutation(s) as described above in any of the VH, VL, CL, CH1 and/or CH2 domains, whereas an antibody construct that comprises only an Fc region, such as an Fc fusion protein, may comprise cysteine insertion mutation(s) as described above in the CH2 domain. Similarly, an antibody construct that comprises an antigen binding domain, such as an scFv or a Fab, but lacks an Fc region, may comprise cysteine insertion mutation(s) in the VH, VL, CL and/or CH1 domains.

In some embodiments, the cysteine engineered antibody construct comprises a cysteine insertion mutation in the Fc region selected from:
(a) an insertion of a cysteine residue between positions 237 and 238 in the CH2 domain;
(b) an insertion of a cysteine residue between positions 295 and 296 in the CH2 domain, and
(c) an insertion of a cysteine residue between positions 299 and 300 in the CH2 domain.

In some embodiments, the cysteine engineered antibody construct comprises a cysteine insertion mutation in the Fab region selected from:
(a) an insertion of a cysteine residue between positions 39 and 40 in the VL domain;
(b) an insertion of a cysteine residue between positions 40 and 41 in the VL domain;
(c) an insertion of a cysteine residue between positions 126 and 127 in the CL domain;
(d) an insertion of a cysteine residue between positions 148 and 149 in the CL domain;
(e) an insertion of a cysteine residue between positions 149 and 150 in the CL domain;
(f) an insertion of a cysteine residue between positions 9 and 10 in the VH domain, and
(g) an insertion of a cysteine residue between positions 169 and 170 in the CH1 domain.

In some embodiments, the cysteine engineered antibody construct comprises a cysteine insertion mutation in the CL domain or CH1 domain selected from:
(a) an insertion of a cysteine residue between positions 126 and 127 in the CL domain;
(b) an insertion of a cysteine residue between positions 148 and 149 in the CL domain;
(c) an insertion of a cysteine residue between positions 149 and 150 in the CL domain, and
(d) an insertion of a cysteine residue between positions 169 and 170 in the CH1 domain.

In some embodiments, the cysteine engineered antibody construct comprises a cysteine insertion mutation in the variable region selected from:
(a) an insertion of a cysteine residue between positions 39 and 40 in the VL domain;
(b) an insertion of a cysteine residue between positions 40 and 41 in the VL domain, and
(c) an insertion of a cysteine residue between positions 9 and 10 in the VH domain.

In some embodiments, the cysteine engineered antibody construct comprises a cysteine insertion mutation as described above in the CH2 domain or in the variable region. In some embodiments, the cysteine engineered antibody construct comprises a cysteine insertion mutation as described above in the CH2 domain or in the variable region, where the cysteine insertion mutation is selected from:
(a) an insertion of a cysteine residue between positions 40 and 41 in the VL domain;
(b) an insertion of a cysteine residue between positions 9 and 10 in the VH domain;
(c) an insertion of a cysteine residue between positions 237 and 238 in the CH2 domain, and
(d) an insertion of a cysteine residue between positions 299 and 300 in the CH2 domain.

The cysteine insertion mutations described herein may be introduced into an antibody construct symmetrically (i.e. the same cysteine insertion mutation is introduced into each respective heavy chain or light chain) or they may be introduced asymmetrically (i.e. one cysteine insertion mutation is introduced into one heavy or light chain and a different cysteine insertion mutation, or no cysteine insertion mutation, is introduced into the other heavy or light chain). In certain embodiments, the cysteine engineered antibody constructs comprise symmetric cysteine insertion mutations. In some embodiments, the cysteine engineered antibody constructs comprise one or more asymmetric cysteine insertion mutations. In some embodiments, the cysteine engineered antibody constructs comprise a combination of symmetric and asymmetric cysteine insertion mutations.

Introducing two cysteine insertion mutations symmetrically into an antibody construct, for example into both heavy chains or both light chains, results in a cysteine engineered antibody construct that when conjugated to an active agent has an average drug-to-antibody ratio (DAR) of 2. Introducing asymmetrical cysteine insertion mutations and/or combinations of cysteine insertion mutations into an antibody construct allows the DAR of the final conjugate to be “tuned.” For example, antibody constructs which comprise a cysteine insertion mutation in only one chain of the construct allows for DAR 1 conjugates, and antibody constructs which comprise a combination of cysteine insertion mutations allows for conjugates having DAR≥2. In those embodiments in which the cysteine engineered antibody construct comprises a combination of cysteine insertion mutations, the mutations may be introduced symmetrically (i.e. the same cysteine insertion mutations are included in both chains of the antibody construct), asymmetrically (i.e. the cysteine insertion mutation or mutations in one chain of the antibody construct are different to or absent from the other chain of the antibody construct), or a combination thereof (i.e. at least one cysteine insertion mutation in one chain of the antibody construct is the same as a cysteine insertion mutation in the other chain of the antibody construct, and at least one cysteine insertion mutation is different or absent from the other chain). Typically, when the antibody construct comprises a single cysteine insertion mutation or asymmetric cysteine insertion mutations, the cysteine insertion mutation(s) are introduced into the heavy chain of the antibody construct. However, asymmetrical light chain cysteine insertion mutations are contemplated in certain embodiments.

Certain embodiments of the present disclosure relate to cysteine engineered antibody constructs comprising two cysteine insertion mutations that are symmetrical (i.e. each inserted cysteine residue is at the same position on each respective heavy or light chain).

Certain embodiments of the present disclosure relate to “DAR-tuned” cysteine engineered antibody constructs that comprise one or a combination of the cysteine insertion mutations described herein. In some embodiments, the cysteine engineered antibody construct comprises between 1 and 8 cysteine insertion mutations. In some embodiments, the cysteine engineered antibody construct comprises between 1 and 6 cysteine insertion mutations. In some embodiments, the cysteine engineered antibody construct comprises between 1 and 4 cysteine insertion mutations.

Certain embodiments of the present disclosure relate to a DAR-tuned cysteine engineered antibody construct comprising an odd number of cysteine insertion mutations, for example, 1, 3, 5 or 7 cysteine insertion mutations. Some embodiments relate to a DAR-tuned cysteine engineered antibody construct comprising 1, 3 or 5 cysteine insertion mutations. Some embodiments relate to a DAR-tuned cysteine engineered antibody construct comprising 1 or 3 cysteine insertion mutations.

Certain embodiments relate to a cysteine engineered antibody construct that comprises a single (1) cysteine insertion mutation. In some embodiments, the cysteine engineered antibody construct comprises a single cysteine insertion mutation in a heavy chain of the antibody construct. In some embodiments, the cysteine engineered antibody construct comprises a single cysteine insertion mutation selected from:
(a) an insertion of a cysteine residue between positions 9 and 10 in the VH domain;
(b) an insertion of a cysteine residue between positions 169 and 170 in the CH1 domain;
(c) an insertion of a cysteine residue between positions 237 and 238 in the CH2 domain;
(d) an insertion of a cysteine residue between positions 295 and 296 in the CH2 domain, and
(e) an insertion of a cysteine residue between positions 299 and 300 in the CH2 domain.

In some embodiments, the cysteine engineered antibody construct comprises a single cysteine insertion mutation selected from:
(a) an insertion of a cysteine residue between positions 9 and 10 in the VH domain;
(b) an insertion of a cysteine residue between positions 237 and 238 in the CH2 domain, and
(c) an insertion of a cysteine residue between positions 299 and 300 in the CH2 domain.

Certain embodiments of the present disclosure relate to a cysteine engineered antibody construct that comprises three cysteine insertion mutations as described herein. In such embodiments, the cysteine engineered antibody construct may comprise three different (asymmetric) cysteine insertion mutations, or it may comprise two symmetric cysteine insertion mutations (i.e. at the same position on each respective heavy or light chain) and one asymmetric cysteine insertion (one inserted cysteine residue on one light or heavy chain). In some embodiments, the cysteine engineered antibody construct comprises 3 cysteine insertion mutations, two of which are the same (symmetric) and one which is different (asymmetric).

In certain embodiments, the cysteine engineered antibody construct comprises 3 cysteine insertion mutations, two of which are the same (symmetric) and one which is different (asymmetric), where the symmetric cysteine insertion mutations are selected from:
(a) an insertion of a cysteine residue between positions 39 and 40 in the VL domain;
(b) an insertion of a cysteine residue between positions 40 and 41 in the VL domain;
(c) an insertion of a cysteine residue between positions 126 and 127 in the CL domain;
(d) an insertion of a cysteine residue between positions 148 and 149 in the CL domain;
(e) an insertion of a cysteine residue between positions 149 and 150 in the CL domain;
(f) an insertion of a cysteine residue between positions 9 and 10 in the VH domain;
(g) an insertion of a cysteine residue between positions 169 and 170 in the CH1 domain;
(h) an insertion of a cysteine residue between positions 237 and 238 in the CH2 domain;
(i) an insertion of a cysteine residue between positions 295 and 296 in the CH2 domain, and
(j) an insertion of a cysteine residue between positions 299 and 300 in the CH2 domain,

and the asymmetric cysteine insertion mutation is selected from:
(i) an insertion of a cysteine residue between positions 9 and 10 in the VH domain;
(ii) an insertion of a cysteine residue between positions 169 and 170 in the CH1 domain;
(iii) an insertion of a cysteine residue between positions 237 and 238 in the CH2 domain;
(iv) an insertion of a cysteine residue between positions 295 and 296 in the CH2 domain, and
(v) an insertion of a cysteine residue between positions 299 and 300 in the CH2 domain.

In certain embodiments, the cysteine engineered antibody construct comprises 3 cysteine insertion mutations, two of which are the same (symmetric) and one which is different (asymmetric), where the symmetric cysteine insertion mutations are selected from:
(a) an insertion of a cysteine residue between positions 40 and 41 in the VL domain;
(b) an insertion of a cysteine residue between positions 9 and 10 in the VH domain, and
(c) an insertion of a cysteine residue between positions 299 and 300 in the CH2 domain, and the asymmetric cysteine insertion mutation is selected from:
(i) an insertion of a cysteine residue between positions 9 and 10 in the VH domain;
(ii) an insertion of a cysteine residue between positions 237 and 238 in the CH2 domain, and
(iii) an insertion of a cysteine residue between positions 299 and 300 in the CH2 domain.

Certain embodiments of the present disclosure relate to a DAR-tuned cysteine engineered antibody construct comprising an even number of cysteine insertion mutations, for example, 4, 6 or 8 cysteine insertion mutations. Some embodiments relate to a DAR-tuned cysteine engineered antibody construct comprising 4 or 6 cysteine insertion mutations. Typically, in such embodiments, the cysteine insertion mutations are symmetric cysteine insertion mutations. However, asymmetric cysteine insertion mutations are also contemplated in some embodiments.

In certain embodiments, the cysteine engineered antibody construct comprises 4, 6 or 8 cysteine insertion mutations, where the cysteine insertion mutations are selected from:
(i) an insertion of a cysteine residue between positions 40 and 41 in the VL domain;
(ii) an insertion of a cysteine residue between positions 126 and 127 in the CL domain;
(iii) an insertion of a cysteine residue between positions 9 and 10 in the VH domain;
(iv) an insertion of a cysteine residue between positions 237 and 238 in the CH2 domain, and
(v) an insertion of a cysteine residue between positions 299 and 300 in the CH2 domain.

In certain embodiments, the cysteine engineered antibody construct comprises 4 or 6 cysteine insertion mutations, where the cysteine insertion mutations are selected from:
(i) an insertion of a cysteine residue between positions 40 and 41 in the VL domain;
(ii) an insertion of a cysteine residue between positions 126 and 127 in the CL domain; (iii) an insertion of a cysteine residue between positions 9 and 10 in the VH domain;
(iv) an insertion of a cysteine residue between positions 237 and 238 in the CH2 domain, and
(v) an insertion of a cysteine residue between positions 299 and 300 in the CH2 domain.

### Additional Mutations

In certain embodiments of the present disclosure, the cysteine engineered antibody constructs may comprise additional mutations known in art to provide a desired change in functionality to the antibody construct. For example, in some embodiments mutations may be introduced into the CH2 domain of the cysteine engineered antibody construct to alter binding to one or more Fc receptors and/or mutations may be introduced into the CH3 domain of the cysteine engineered antibody construct to improve heterodimer formation when the antibody construct comprises a heterodimeric Fc region. In some embodiments in which the antibody construct is bispecific or multispecific, mutations may also be introduced into the Fab regions in order to promote correct pairing between each heavy chain and light chain. Examples of such Fab region mutations include those described in International Patent Application Publication Nos. WO 2014/082179, WO 2015/181805 and WO 2017/059551.

### CH2 Domain Mutations

In certain embodiments, the cysteine engineered antibody construct may comprise one or more additional mutations in the CH2 domain, for example, the cysteine engineered antibody construct may comprise a modified CH2 domain having altered binding to one or more Fc receptors, such as receptors of the FcγRI, FcγRII and FcγRIII subclasses.

Various amino acid mutations to the CH2 domain that selectively alter affinity for different Fcγ receptors are known in art. Amino acid mutations that result in increased binding and amino acid modifications that result in decreased binding can both be useful in certain indications. For example, increasing binding affinity of an Fc for FcγRIIIa (an activating receptor) results in increased antibody dependent cell-mediated cytotoxicity (ADCC), which in turn results in increased lysis of the target cell. Decreased binding to FcγRIIb (an inhibitory receptor) likewise may be beneficial in some circumstances. Increased binding to FcγRIIb, or decreased or eliminated binding of the Fc region to all of the Fcγ receptors (“knock-out” variants) may be useful when a decrease in, or elimination of, ADCC and complement-mediated cytotoxicity (CDC) is desirable.

Examples of amino acid mutations that alter binding by Fcγ receptors include, but are not limited to, S298A/E333A/K334A and S298A/E333A/K334A/K326A (increased affinity for FcγRIIIa) (Lu, et al., 2011, J Immunol Methods, 365 (1-2): 132-41); F243L/R292P/Y300L/V305I/P396L (increased affinity for FcγRIIIa) (Stavenhagen, et al., 2007, Cancer Res, 67 (18): 8882-90); F243L/R292P/Y300L/L235V/P396L (increased affinity for FcγRIIIa) (Nordstrom, et al., 2011, Breast Cancer Res, 13 (6): R123); F243L (increased affinity for FcγRIIIa) (Stewart, et al., 2011, Protein Eng Des Sel., 24 (9): 671-8); S298A/E333A/K334A (increased affinity for FcγRIIIa) (Shields, et al., 2001, J Biol Chem, 276 (9): 6591-604); S239D/1332E/A330L and S239D/1332E (increased affinity for FcγRIIIa) (Lazar, et al., 2006, Proc Natl Acad Sci USA, 103 (11): 4005-10), and S239D/S267E and S267E/L328F (increased affinity for FcγRIIb) (Chu, et al., 2008, Mol Immunol, 45 (15): 3926-33).

Additional modifications that affect Fc binding to Fcγ receptors are described in Therapeutic Antibody Engineering (Strohl & Strohl, Woodhead Publishing series in Biomedicine No 11, ISBN 1 907568 37 9, October 2012, page 283).

Various publications describe strategies that have been used to engineer antibodies to produce “knock-out” variants (see, for example, Strohl, 2009, Curr Opin Biotech 20:685-691; Strohl & Strohl, “Antibody Fc engineering for optimal antibody performance” In Therapeutic Antibody Engineering, Cambridge: Woodhead Publishing, 2012, pp 225-249). These strategies include reduction of effector function through modification of glycosylation, use of IgG2/IgG4 scaffolds, or the introduction of mutations in the hinge or CH2 domain of the Fc (see also, U.S. Patent Publication Nos. 2011/0212087, 2012/0225058 and 2012/0251531, International Publication No. WO 2006/105338, and Strop et al., 2012, J. Mol. Biol., 420:204-219).

Specific, non-limiting examples of known amino acid mutations to reduce FcγR and/or complement binding to the Fc include, but are not limited to, N297A; L234A/L235A; C220S/C226S/C229S/P238S; C226S/C229S/E3233P/L235V/L235A; L234F/L235E/P331S; IgG2 V234A/G237A; IgG2 H268Q/V309L/A330S/A331S; IgG4 L235A/G237A/E318A and IgG4 S228P/L236E. Additional examples include Fc regions engineered to include the amino acid modifications L235A/L236A/D265S, and the asymmetric amino acid modifications described in International Patent Application Publication No. WO 2014/190441.

### CH3 Domain Mutations

In certain embodiments, the cysteine engineered antibody constructs described herein may comprise one or more additional mutations in the CH3 domain, for example, the cysteine engineered antibody constructs may comprise a modified CH3 domain comprising one or more amino acid mutations that promote formation of a heterodimeric Fc over formation of a homodimeric Fc. Heterodimeric Fc regions can be useful, for example, in bispecific antibody constructs and in those cysteine engineered antibody constructs comprising a single cysteine insertion mutation or asymmetric combinations of cysteine insertion mutations.

Various amino acid mutations that may be made to the CH3 domain of an Fc in order to promote formation of a heterodimeric Fc are known in art and include, for example, those described in International Patent Application Publication No. WO 96/027011 (“knobs into holes”), Gunasekaran et al., 2010, J Biol Chem, 285, 19637-46 (“electrostatic steering”), Davis et al., 2010, Prot Eng Des Sel, 23 (4): 195-202 (strand exchange engineered domain (SEED) technology) and Labrijn et al., 2013, Proc Natl Acad Sci USA, 110 (13): 5145-50 (Fab-arm exchange). Other examples include asymmetrically modified Fc regions as described in International Patent Application Publication Nos. WO 2012/058768 and WO 2013/063702.

In certain embodiments, the cysteine engineered antibody construct comprises a modified CH3 domain in which one Fc polypeptide comprises an amino acid mutation at position F405 selected from F405A, F405S, F405T and F405V, and an amino acid mutation at position Y407 selected from Y4071 and Y407V, and the other Fc polypeptide comprises an amino acid mutation at position T366 selected from T366I, T366L or T366M, and the amino acid mutation T394W. In some embodiments, the amino acid mutation at position T366 is T3661 or T366L.

In some embodiments, one Fc polypeptide comprises amino acid mutations at positions F405 and Y407 as described above, and further includes the amino acid mutation L351Y.

In some embodiments, one Fc polypeptide comprises amino acid mutations at positions T366 and T394 as described above, and further includes an amino acid mutation at position K392 selected from K392F, K392L or K392M. In some embodiments, the amino acid mutation at position K392 is K392L or K392M.

In some embodiments, the cysteine engineered antibody construct comprises a modified CH3 domain as described above in which one Fc polypeptide comprises amino acid mutations at positions F405 and Y407, and optionally further comprises an amino acid mutation at position L351, and the other Fc polypeptide comprises amino acid mutations at positions T366 and T394, and optionally further comprises an amino acid mutation at position K392, and one or both of the Fc polypeptides further comprises the amino acid mutation T350V.

In certain embodiments, the cysteine engineered antibody construct comprises a modified CH3 domain in which one Fc polypeptide comprises the amino acid mutation F405A, F405S, F405T or F405V together with the amino acid mutation Y4071 or Y407V, and optionally further includes the amino acid mutation L351Y, and the other Fc polypeptide comprises the amino acid mutation T3661 or T366L, together with the amino acid mutation T394W, and optionally further includes the amino acid mutation K392L or K392M. In some embodiments, one or both of the Fc polypeptides further comprises the amino acid mutation T350V. In some embodiments, both Fc polypeptides further comprise the amino acid mutation T350V.

In certain embodiments, the cysteine engineered antibody construct comprises a modified CH3 domain comprising the amino acid mutations as set forth for any one of Variant 1, Variant 2, Variant 3, Variant 4 or Variant 5 in Table 1.

TABLE 1
Modified CH3 Domains

| Variant # | Chain | Mutations |
|---|---|---|
| 1 | A | L351Y_F405A_Y407V |
| | B | T366L_K392M_T394W |
| 2 | A | L351Y_F405A_Y407V |
| | B | T366L_K392L_T394W |
| 3 | A | T350V_L351Y_F405A_Y407V |
| | B | T350V_T366L_K392L_T394W |
| 4 | A | T350V_L351Y_F405A_Y407V |
| | B | T350V_T366L_K392M_T394W |
| 5 | A | T350V_L351Y_S400E_F405A_Y407V |
| | B | T350V_T366L_N390R_K392M_T394W |

### Preparation of Cysteine Engineered Antibody Constructs

The cysteine engineered antibody constructs described herein may be prepared using standard recombinant methods. Recombinant production generally involves synthesizing one or more polynucleotides encoding the cysteine engineered antibody construct, cloning the one or more polynucleotides into an appropriate vector or vectors, and introducing the vector(s) into a suitable host cell for expression of the cysteine engineered antibody construct. Recombinant production of proteins is well-known in art and may be achieved using standard techniques as described, for example, in Sambrook et al., Molecular Cloning: A Laboratory Manual, 3rd ed., Cold Spring Harbor Laboratory Press, Cold Spring Harbor, NY (2001); Ausubel et al., Current Protocols in Molecular Biology, (1987 & updates), John Wiley & Sons, New York, NY; and Harlow and Lane, Antibodies: A Laboratory Manual, Cold Spring Harbor Laboratory Press, Cold Spring Harbor, NY (1990).

Certain embodiments of the present disclosure thus relate to an isolated polynucleotide or set of polynucleotides encoding a cysteine engineered antibody construct as described herein. A polynucleotide in this context thus may encode all or part of a cysteine engineered antibody construct.

The terms “polynucleotide,” “nucleic acid” and “nucleic acid molecule” are used interchangeably herein and refer to a polymeric form of nucleotides of any length, either deoxyribonucleotides or ribonucleotides, or analogs thereof. A polynucleotide that “encodes” a given polypeptide is a polynucleotide that is transcribed (in the case of DNA) and translated (in the case of mRNA) into a polypeptide in vivo when placed under the control of appropriate regulatory sequences. The boundaries of the coding sequence are determined by a start codon at the 5′ (amino) terminus and a translation stop codon at the 3′ (carboxy) terminus. A transcription termination sequence may be located 3′ to the coding sequence.

For expression of the cysteine engineered antibody construct, one or more polynucleotides encoding the cysteine engineered antibody construct may be inserted into a suitable expression vector, either directly or after one or more subcloning steps, using standard ligation techniques. Examples of suitable vectors include, but are not limited to, plasmids, phagemids, cosmids, bacteriophage, baculoviruses, retroviruses or DNA viruses. The vector is typically selected to be functional in the particular host cell that will be employed, i.e. the vector is compatible with the host cell machinery, permitting amplification and/or expression of the polynucleotide(s). Selection of appropriate vector and host cell combinations in this regard is well within the ordinary skills of a worker in art.

Certain embodiments of the present disclosure thus relate to vectors (such as expression vectors) comprising one or more polynucleotides encoding a cysteine engineered antibody construct as described herein. The polynucleotide(s) may be comprised by a single vector or by more than one vector. In some embodiments, the polynucleotides are comprised by a multicistronic vector.

Typically, expression vectors will contain one or more regulatory elements for plasmid maintenance and for cloning and expression of exogenous polynucleotide sequences. Examples of such regulatory elements include promoters, enhancer sequences, origins of replication, transcriptional termination sequences, donor and acceptor splice sites, leader sequences for polypeptide secretion, ribosome binding sites, polyadenylation sequences, polylinker regions for inserting the polynucleotide encoding the polypeptide to be expressed, and selectable markers.

Regulatory elements may be homologous (i.e. from the same species and/or strain as the host cell), heterologous (i.e. from a species other than the host cell species or strain), hybrid (i.e. a combination of regulatory sequences from more than one source) or synthetic. As such, the source of a regulatory sequence may be any prokaryotic or eukaryotic organism provided that the regulatory sequence is functional in, and can be activated by, the machinery of the host cell being employed.

Optionally, the vector may contain a “tag”-encoding sequence, that is a nucleic acid sequence located at the 5′ or 3′ end of the coding sequence that encodes a heterologous peptide sequence, such as a polyHis (for example, 6xHis), FLAGR, HA (hemaglutinin influenza virus), myc, metal-affinity, avidin/streptavidin, glutathione-S-transferase (GST) or biotin tag. This tag typically remains fused to the expressed protein and can serve as a means for affinity purification or detection of the protein. Optionally, the tag can subsequently be removed from the purified protein by various means, for example, by using certain peptidases for cleavage.

Various expression vectors are readily available from commercial sources. Alternatively, when a commercial vector containing all the desired regulatory elements is not available, an expression vector may be constructed using a commercially available vector as a starting vector. Where one or more of the desired regulatory elements are not already present in the vector, they may be individually obtained and ligated into the vector. Methods for obtaining various regulatory elements and constructing expression vectors are well known to one skilled in art.

Once the expression vector including the polynucleotide(s) encoding the cysteine engineered antibody construct has been constructed, the vector may be inserted into a suitable host cell for amplification and/or protein expression. The transformation of an expression vector into a selected host cell may be accomplished by well-known methods including transfection, infection, calcium phosphate co-precipitation, electroporation, microinjection, lipofection, DEAE-dextran mediated transfection, and other known techniques. The method selected will in part be dependent on the type of host cell to be used. These methods and other suitable methods are well known to the skilled person (see, for example, Sambrook, et al., ibid.).

A host cell transformed with the expression vector, when cultured under appropriate conditions, expresses the protein encoded by the vector and the protein can subsequently be collected from the culture medium (if the host cell secretes the protein) or directly from the host cell producing it (if the protein is not secreted). The host cell may be prokaryotic (for example, a bacterial cell) or eukaryotic (for example, a yeast, fungi, plant or mammalian cell). The selection of an appropriate host cell can be readily made by the skilled person taking into account various factors, such as desired expression levels, polypeptide modifications that are desirable or necessary for activity (such as glycosylation or phosphorylation) and ease of folding into a biologically active molecule.

Certain embodiments of the present disclosure thus relate to host cells comprising polynucleotide(s) encoding a cysteine engineered antibody construct or one or more vectors comprising the polynucleotide(s) encoding the cysteine engineered antibody construct. In certain embodiments, the host cell is a eukaryotic cell.

For example, eukaryotic microbes such as filamentous fungi or yeast may be employed as host cells, including fungi and yeast strains whose glycosylation pathways have been “humanized” (see, for example, Gerngross, 2004, Nat. Biotech., 22:1409-1414, and Li et al., 2006, Nat. Biotech., 24:210-215). Plant cells may also be utilized as host cells (see, for example, U.S. Pat. Nos. 5,959,177; 6,040,498; 6,420,548; 7,125,978 and 6,417,429, describing PLANTIBODIES™ technology).

In some embodiments, the host cell is a mammalian cell. Various mammalian cell lines may be used as host cells. Examples of useful mammalian host cell lines include, but are not limited to, monkey kidney CV1 line transformed by SV40 (COS-7), human embryonic kidney line 293 (HEK293 cells as described, for example, in Graham, et al., 1977, J. Gen Virol., 36:59), baby hamster kidney cells (BHK), mouse sertoli cells (TM4 cells as described, for example, in Mather, 1980, Biol. Reprod., 23:243-251), monkey kidney cells (CV1), African green monkey kidney cells (VERO-76), human cervical carcinoma cells (HeLa), canine kidney cells (MDCK), buffalo rat liver cells (BRL 3A), human lung cells (W138), human liver cells (Hep G2), mouse mammary tumour (MMT 060562), TRI cells (as described, for example, in Mather, et al., 1982, Annals N.Y. Acad. Sci., 383:44-68), MRC 5 cells, FS4 cells, Chinese hamster ovary (CHO) cells (including DHFR CHO cells as described in Urlaub, et al., 1980, Proc. Natl. Acad. Sci. USA, 77:4216) and myeloma cell lines (such as YO, NSO and Sp2/0). See also, Yazaki and Wu, 2003, Methods in Molecular Biology, Vol. 248, pp. 255-268 (B.K.C. Lo, ed., Humana Press, Totowa, N.J.).

Certain embodiments of the present disclosure relate to methods of preparing a cysteine engineered antibody construct as described herein, comprising transfecting a host cell with one or more polynucleotides encoding the cysteine engineered antibody construct, for example as one or more vectors comprising the polynucleotide(s), and culturing the host cell under conditions suitable for expression of the encoded cysteine engineered antibody construct.

Typically, the cysteine engineered antibody construct is isolated from the host cell after expression and may optionally be purified. Methods for isolating and purifying expressed proteins are well-known in art. Standard purification methods include, for example, chromatographic techniques, such ion exchange, hydrophobic interaction, affinity, sizing, gel filtration or reversed-phase, which may be carried out at atmospheric pressure or at medium or high pressure using systems such as FPLC, MPLC and HPLC. Other purification methods include electrophoretic, immunological, precipitation, dialysis, and chromatofocusing techniques. Ultrafiltration and diafiltration techniques, in conjunction with protein concentration, may also be useful.

A variety of natural proteins are known in art to bind Fc regions or other regions of antibodies, and these proteins can therefore be used in the purification of Fc-containing proteins. For example, the bacterial proteins A and G bind to the Fc region. Likewise, the bacterial protein L binds to the Fab region of some antibodies. Purification can often be enabled by a particular fusion partner or affinity tag as described above. For example, antibodies may be purified using glutathione resin if a GST fusion is employed, Ni 2 affinity chromatography if a His-tag is employed, or immobilized anti-flag antibody if a flag-tag is used. Examples of useful purification techniques are described in Harlow and Lane, Antibodies: A Laboratory Manual, Cold Spring Harbor Laboratory Press, Cold Spring Harbor, NY (1990), and Protein Purification: Principles and Practice, 3rd Ed., Scopes, Springer-Verlag, NY (1994).

### Conjugates

Certain embodiments of the present disclosure relate to conjugates comprising a cysteine engineered antibody construct as described herein and an active agent conjugated to the antibody construct via an inserted cysteine residue. The active agent may be, for example, a therapeutic agent, a diagnostic agent or a labelling agent.

Conjugation of the selected active agent to a cysteine engineered antibody construct can be accomplished in a variety of ways known in art and may be direct or via a linker. Linkers for conjugation of active agents are bifunctional or multifunctional moieties capable of linking one or more active agents to an antibody construct. A bifunctional (or monovalent) linker links a single active agent to a single site on the antibody construct, whereas a multifunctional (or multivalent) linker links more than one active agent to a single site on the antibody construct. Linkers capable of linking one active agent to more than one site on the antibody construct may also be considered to be multifunctional.

When a linker is employed to conjugate an active agent to the cysteine engineered antibody construct, the linker comprises a thiol-reactive functional group allowing it to react with an inserted cysteine residue in antibody construct. Examples of thiol-reactive functional groups include, but are not limited to, maleimide, α-haloacetyl, activated esters such as succinimide esters, 4-nitrophenyl esters, pentafluorophenyl esters, tetrafluorophenyl esters, anhydrides, acid chlorides, sulfonyl chlorides, isothiocyanates and isocyanates.

The linker also includes a functional group capable of reacting with a target group on the active agent. Suitable functional groups are known in art and include those described, for example, in Bioconjugate Techniques (G.T. Hermanson, 2013, Academic Press). Groups on the active agent that may serve as target groups for linker attachment include, but are not limited to, thiol, hydroxyl, carboxyl, amine, aldehyde and ketone groups.

Non-limiting examples of functional groups for reacting with thiols are described above. Non-limiting examples of functional groups for reacting with amines include activated esters (such as N-hydroxysuccinamide (NHS) esters and sulfo-NHS esters), imido esters (such as Traut's reagent), isothiocyanates, aldehydes and acid anhydrides (such as diethylenetriaminepentaacetic anhydride (DTPA)). Other examples include succinimido-1,1,3,3-tetra-methyluronium tetrafluoroborate (TSTU) and benzotriazol-1-yl-oxytripyrrolidinophosphonium hexafluorophosphate (PyBOP).

Non-limiting examples of functional groups capable of reacting with an electrophilic group on the active agent (such as an aldehyde or ketone carbonyl group) include hydrazide, oxime, amino, hydrazine, thiosemicarbazone, hydrazine carboxylate and arylhydrazide.

Linkers may be cleavable or non-cleavable. A cleavable linker is typically susceptible to cleavage under intracellular conditions, for example, through lysosomal processes. Examples include linkers that are protease-sensitive, acid-sensitive or reduction-sensitive. Non-cleavable linkers by contrast, rely on the degradation of the antibody in the cell, which typically results in the release of an amino acid-linker-active agent moiety.

Suitable cleavable linkers include, for example, peptide-containing linkers cleavable by an intracellular protease, such as lysosomal protease or an endosomal protease. For example, the linker may include a dipeptide, such as a valine-citrulline (Val-Cit) or a phenylalanine-lysine (Phe-Lys). Other examples of suitable dipeptides for inclusion in linkers include Val-Lys, Ala-Lys, Phe-Lys, Val-Cit, Phe-Cit, Leu-Cit, Ile-Cit, Trp-Cit, Phe-Arg, Ala-Phe, Val-Ala, Met-Lys, Asn-Lys, Ile-Pro, Ile-Val, Asp-Val, His-Val, Met-(D) Lys, Asn-(D) Lys, Val-(D) Asp, NorVal-(D) Asp, Ala-(D) Asp, Me3Lys-Pro, PhenylGly-(D) Lys, Met-(D) Lys, Asn-(D) Lys, Pro-(D) Lys and Met-(D) Lys. Linkers may also include longer peptide sequences, such as the tripeptides Met-Cit-Val, Gly-Cit-Val, (D) Phe-Phe-Lys or (D) Ala-Phe-Lys, or the tetrapeptides Gly-Phe-Leu-Gly, Gly-Gly-Phe-Gly or Ala-Leu-Ala-Leu.

Additional examples of cleavable linkers include disulfide-containing linkers. Examples of disulfide-containing linkers include, but are not limited to, N-succinimydyl-4-(2-pyridyldithio) butanoate (SPBD) and N-succinimydyl-4-(2-pyridyldithio)-2-sulfo butanoate (sulfo-SPBD). Disulfide-containing linkers may optionally include additional groups to provide steric hindrance adjacent to the disulfide bond in order to improve the extracellular stability of the linker, for example, inclusion of a geminal dimethyl group. Other suitable linkers include linkers hydrolyzable at a specific pH or within a pH range, such as hydrazone linkers.

A further example of a cleavable linker is a linker comprising a β-glucuronide, which is cleavable by β-glucuronidase, an enzyme present in lysosomes and tumor interstitium (see, for example, De Graaf, et al., 2002, Curr. Pharm. Des. 8:1391-1403).

Cleavable linkers may optionally further comprise one or more additional functionalities such as self-immolative and self-elimination groups, stretchers or hydrophilic moieties.

Self-immolative and self-elimination groups that find use in linkers include, for example, p-aminobenzyloxycarbonyl(PAB or PABC) and p-aminobenzyl ether (PABE) groups, and methylated ethylene diamine (MED). Other examples of self-immolative groups include, but are not limited to, aromatic compounds that are electronically similar to the PABC or PABE group such as heterocyclic derivatives, for example 2-aminoimidazol-5-methanol derivatives as described in U.S. Pat. No. 7,375,078. Other examples include groups that undergo cyclization upon amide bond hydrolysis, such as substituted and unsubstituted 4-aminobutyric acid amides (Rodrigues, et al., 1995, Chemistry Biology 2:223-227) and 2-aminophenylpropionic acid amides (Amsberry, et al., 1990, J. Org. Chem. 55:5867-5877). Self-immolative/self-elimination groups, alone or in combination are often included in peptide-based linkers, and may also be included in other types of linkers.

Stretchers that find use in linkers for ADCs include, for example, alkylene groups and stretchers based on aliphatic acids, diacids, amines or diamines, such as diglycolate, malonate, caproate and caproamide. Other stretchers include, for example, glycine-based stretchers and polyethylene glycol (PEG) or monomethoxy polyethylene glycol (mPEG) based stretchers.

Various non-cleavable linkers are also known in art for linking active agents to antibodies. Examples include, but are not limited to, linkers based on N-succinimidyl 4-(maleimidomethyl)cyclohexanecarboxylate (SMCC), sulfosuccinimidyl-4-[N-maleimidomethyl]cyclohexane-1-carboxylate (sulfo-SMCC), N-succinimidyl-4-(N-maleimidomethyl)-cyclohexane-1-carboxy-(6-amidocaproate) (“long chain” SMCC or LC-SMCC), κ-maleimidoundecanoic acid N-succinimidyl ester (KMUA), γ-maleimidobutyric acid N-succinimidyl ester (GMBS), ¿-maleimidocaproic acid N-hydroxysuccinimide ester (EMCS), m-maleimidobenzoyl-N-hydroxysuccinimide ester (MBS), N-(α-maleimidoacetoxy)-succinimide ester (AMAS), succinimidyl-6-(β-maleimidopropionamido) hexanoate (SMPH), N-succinimidyl 4-(p-maleimidophenyl)-butyrate (SMPB), N-(p-maleimidophenyl) isocyanate (PMPI), N-succinimidyl-4-(iodoacetyl)-aminobenzoate (SIAB), N-succinimidyl iodoacetate (SIA), N-succinimidyl bromoacetate (SBA) and N-succinimidyl 3-(bromoacetamido) propionate (SBAP).

The number of active agent molecules that may be conjugated to a given cysteine engineered antibody construct (drug-to-antibody ratio or DAR) will depend on the number of cysteine insertion mutations comprised by the antibody construct and on the type of linker employed (monovalent or multivalent).

Certain embodiments of the present disclosure relate to a conjugate having Formula (I):

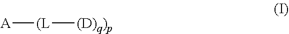

wherein:
A is a cysteine engineered antibody construct as described herein;
L is a linker (for example, a linker as described in any one of the embodiments described above);
D is an active agent;
q is an integer between 1 and 4, and
p is an integer between 1 and 8,
where D is linked to the inserted cysteine residue in the cysteine engineered antibody construct via L.

In some embodiments in Formula (I), q is 1, 2 or 3. In some embodiments in Formula (I), q is 1 or 2. In some embodiments in Formula (I), p is an integer between 1 and 6. In some embodiments in Formula (I), p is 1, 2, 3 or 4. In some embodiments in Formula (I), p is 6.

In some embodiments in Formula (I), q is 1, 2 or 3, and p is 1, 2, 3 or 4. In some embodiments in Formula (I), q is 1 or 2, and p is an integer between 1 and 8. In some embodiments in Formula (I), q is 1 or 2, and p is 1, 2, 3 or 4. In some embodiments in Formula (I), q is 1 or 2, and p is 6.

In certain embodiments, the conjugate has Formula (II):

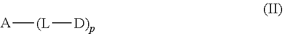

wherein:
A is a cysteine engineered antibody construct as described herein;
L is a linker (for example, a linker as described in any one of the embodiments described above);
D is an active agent, and
p is an integer between 1 and 8,
where D is linked to the inserted cysteine residue in the cysteine engineered antibody construct via L.

In some embodiments in Formula (II), p is an integer between 1 and 6. In some embodiments in Formula (II), p is 1, 2, 3 or 4. In some embodiments in Formula (II), p is 1, 2 or 3. In some embodiments in Formula (II), p is 2. In some embodiments in Formula (II), p is 1 or 3. In some embodiments in Formula (II), p is 4 or 6.

Methods for conjugating various agents to free thiol groups on proteins, including antibodies, are known in art (see, for example, in Bioconjugate Techniques (G.T. Hermanson, 2013, Academic Press) and exemplary methods are also described in the Examples herein.

Certain embodiments of the present disclosure relate to methods of preparing conjugates comprising a cysteine engineered antibody construct of the present disclosure. In some embodiments, the method comprises submitting a cysteine engineered antibody construct comprising at least one inserted cysteine residue as described herein to reducing conditions such that the thiol group of the inserted cysteine residue is reduced, and reacting a thiol reactive linker-active agent with the antibody construct under conditions that permit formation of a bond between the linker and the reduced thiol.

Certain embodiments of the present disclosure relate to methods of preparing an antibody-drug conjugate having a pre-determined drug-to-antibody ratio (DAR), the method comprising reacting a cysteine engineered antibody construct comprising one or more cysteine insertion mutations as described herein with a drug-linker to provide the antibody-drug conjugate, where the pre-determined DAR is 1, 2, 3, 4, 5, 6, 7 or 8, and the cysteine engineered antibody construct comprises the same number of cysteine insertion mutations as the pre-determined DAR. In certain embodiments of this method, the pre-determined DAR is 2. In some embodiments, the pre-determined DAR is 1 or 3. In some embodiments, the pre-determined DAR is 4 or 6.

In some embodiments, the pre-determined DAR is 1 or 3 and the cysteine insertion mutations comprised by the cysteine engineered antibody construct are selected from:
(a) an insertion of a cysteine residue between positions 9 and 10 in the VH domain;
(b) an insertion of a cysteine residue between positions 40 and 41 in the VL domain;
(c) an insertion of a cysteine residue between positions 237 and 238 in the CH2 domain, and
(d) an insertion of a cysteine residue between positions 299 and 300 in the CH2 domain.

In some embodiments, the pre-determined DAR is 1 or 3 and the cysteine insertion mutations comprised by the cysteine engineered antibody construct are selected from:
(i) an insertion of a cysteine residue between positions 40 and 41 in the VL domain;
(ii) an insertion of a cysteine residue between positions 126 and 127 in the CL domain;
(iii) an insertion of a cysteine residue between positions 9 and 10 in the VH domain;
(iv) an insertion of a cysteine residue between positions 237 and 238 in the CH2 domain, and
(v) an insertion of a cysteine residue between positions 299 and 300 in the CH2 domain.

### Active Agents

Active agents that may be conjugated to the cysteine engineered antibody constructs include therapeutic agents, diagnostic agents and labelling agents.

Examples of therapeutic agents include, but are not limited to, antimetabolites, alkylating agents, anthracyclines, antibiotics, anti-mitotic agents, toxins, apoptotic agents, thrombotic agents, anti-angiogenic agents, biological response modifiers, growth factors, radioactive materials and macrocyclic chelators useful for conjugating radiometal ions. Examples of diagnostic agents include, but are not limited to, various imaging agents such as fluorescent materials, luminescent materials and radioactive materials. Examples of labelling agents include, but are not limited to, enzymes, prosthetic groups, fluorescent materials, luminescent materials and radioactive materials.

Certain embodiments of the present disclosure relate to conjugates comprising a cysteine engineered antibody construct as described herein and a therapeutic agent. Some embodiments relate to conjugates comprising a cysteine engineered antibody construct as described herein and an anti-cancer agent. Exemplary anti-cancer agents include, but are not limited to, maytansinoids, auristatins, hemiasterlins, tubulysins, dolastatins, trichothecenes, duocarmycins, camptothecins, calicheamicins and other enediyne antibiotics, taxanes, anthracyclines, Pseudomonas exotoxin (PE), pyrrolobenzodiazapenes (PBD), and analogues and derivatives thereof.

### Pharmaceutical Compositions

Certain embodiments of the present disclosure relate to pharmaceutical compositions for therapeutic or diagnostic use comprising a conjugate as described herein and a pharmaceutically acceptable carrier or diluent. The compositions may be prepared by known procedures using well-known and readily available ingredients and may be formulated for administration to a subject by, for example, oral (including, for example, buccal or sublingual), topical, parenteral, rectal or vaginal routes, or by inhalation or spray. The term “parenteral” as used herein includes injection or infusion by subcutaneous, intradermal, intra-articular, intravenous, intramuscular, intravascular, intrasternal or intrathecal routes.

The composition will typically be formulated in a format suitable for administration to the subject by the chosen route, for example, as a syrup, elixir, tablet, troche, lozenge, hard or soft capsule, pill, suppository, oily or aqueous suspension, dispersible powder or granule, emulsion, injectable or solution. Compositions may be provided as unit dosage formulations.

Pharmaceutically acceptable carriers are generally nontoxic to recipients at the dosages and concentrations employed. Examples of such carriers include, but are not limited to, buffers such as phosphate, citrate, and other organic acids; antioxidants such as ascorbic acid and methionine; preservatives such as octadecyldimethylbenzyl ammonium chloride, hexamethonium chloride, benzalkonium chloride, benzethonium chloride, phenol, butyl alcohol, benzyl alcohol, alkyl parabens (such as methyl or propyl paraben), catechol, resorcinol, cyclohexanol, 3-pentanol and m-cresol; low molecular weight (less than about 10 residues) polypeptides; proteins such as serum albumin or gelatin; hydrophilic polymers such as polyvinylpyrrolidone; amino acids such as glycine, glutamine, asparagine, histidine, arginine or lysine; monosaccharides, disaccharides, and other carbohydrates such as glucose, mannose or dextrins; chelating agents such as EDTA; sugars such as sucrose, mannitol, trehalose or sorbitol; salt-forming counter-ions such as sodium; metal complexes such as Zn-protein complexes, and non-ionic surfactants such as polyethylene glycol (PEG).

In certain embodiments, the compositions may be in the form of a sterile injectable aqueous or oleaginous solution or suspension. Such suspensions may be formulated using suitable dispersing or wetting agents and/or suspending agents that are known in art. The sterile injectable solution or suspension may comprise the conjugate in a non-toxic parentally acceptable diluent or solvent. Acceptable diluents and solvents that may be employed include, for example, 1,3-butanediol, water, Ringer's solution or isotonic sodium chloride solution. In addition, sterile, fixed oils may be employed as a solvent or suspending medium. For this purpose, various bland fixed oils may be employed, including synthetic mono- or diglycerides. In addition, fatty acids such as oleic acid find use in the preparation of injectables. Adjuvants such as local anaesthetics, preservatives and/or buffering agents as known in art may also be included in the injectable solution or suspension.

Other pharmaceutical compositions and methods of preparing pharmaceutical compositions are known in art and are described, for example, in “Remington: The Science and Practice of Pharmacy” (formerly “Remingtons Pharmaceutical Sciences”); Gennaro, A., Lippincott, Williams & Wilkins, Philadelphia, PA (2000).

### Methods of Use

Conjugates comprising a cysteine engineered antibody construct of the present disclosure conjugated to an active agent may be used in methods of treatment, methods of diagnosis and screening methods. The exact nature of the method will be dependent on the nature of the conjugate, including the type of active agent conjugated to the cysteine engineered antibody construct.

For example, certain embodiments of the present disclosure relate to methods of treating a disease or disorder by administering to a subject having the disease or disorder a conjugate comprising a cysteine engineered antibody construct as described herein conjugated to a therapeutic agent. In some embodiments in which the therapeutic agent is an anti-cancer agent, the conjugates may be used in methods of treating cancer.

Certain embodiments of the present disclosure relate to methods of diagnosing a disease or disorder comprising administering to a subject suspected of having, or known to have, the disease or disorder a conjugate comprising a cysteine engineered antibody construct as described herein conjugated to a diagnostic agent. Some embodiments relate to methods of diagnosing a disease or disorder comprising contacting a biological sample taken from a subject suspected of having, or known to have, the disease or disorder with a conjugate comprising a cysteine engineered antibody construct as described herein conjugated to a diagnostic agent.

Certain embodiments of the present disclosure relate to methods of screening a biological sample, such as a sample taken from a subject, for the presence of a target moiety comprising contacting the sample with a conjugate comprising a cysteine engineered antibody construct as described herein conjugated to a labelling agent, where the cysteine engineered antibody construct specifically binds the target moiety.

The following Examples are provided for illustrative purposes and are not intended to limit the scope of the claimed invention in any way.

### Examples

### General Procedures

### 1. Cloning, Expression and Purification of Cysteine Engineered Variants

IgG1 antibodies targeting c-Met or FRα and having a heterodimeric Fc region (HetFc) were used to construct the cysteine engineered constructs and controls described in the following Examples. The HetFc includes the following mutations in the CH3 domain:
Chain A (HC-A): T350V_L351Y_F405A_Y407V
Chain B (HC-B): T350V_T366L_K392L_T394W

The cysteine engineered constructs and controls were cloned and expressed as follows. The genes encoding the antibody heavy and light chains were constructed via gene synthesis using codons optimized for human/mammalian expression. The signal peptide MAVMAPRTLVLLLSGALALTQTWAG [SEQ ID NO:1] was included at the N-terminus of each polypeptide sequence. In some instances, the light chain contained the peptide ESSCDVKLV [SEQ ID NO:2] fused directly to the C-terminal residue.

The final gene products were sub-cloned into the mammalian expression vector PTT5 (NRC-BRI, Canada) and expressed in CHO cells (Durocher, et al., 2002, Nucl Acids Res., 30 (2): E9). Briefly, CHO-3E7 cells were grown in suspension in FreeStyle™ F17 medium (Thermo Fisher Scientific, Waltham, MA) supplemented with 0.1% w/v Pluronic and 4 mM glutamine to a cell density of 1.5-2 million cells/ml with viability ≥97%. Transfection was carried out as described by Durocher and coworkers (Delafosse, et al., 2016, J Biotechnol, 227:103-111; Raymond, et al., 2015, MAbs, 7 (3): 571-83) using a mixture of plasmid DNA: 5% pTTo-GFP plasmid (green fluorescent protein to determine transfection efficiency), 15% pTT22-AKT plasmid, 21% of antibody construct DNA (at ratio 1:1:3 HC-A, HC-B, LC), 68.37% salmon sperm DNA. Following transfection, the shake flask containing cells was placed on an orbital shaker set to 120 rpm in a humidified incubator with 5% CO2 at 37° C. Twenty-four hours post-transfection, 1% w/v tryptone N1 (TN1) and 0.5 mM valproic acid were added to the cultures. The cultures were then transferred to an orbital shaker (120 rpm) placed in a humidified incubator with 5% CO2 at 32° C. At 24-48 hours, GFP positive cells should be between 30-60% as determined by flow cytometry. Cells were harvested 7-10 days post-transfection and spun at 4,000 rpm, then filter-sterilized (clarified) using a 0.45 μm filter (Millipore Sigma, Burlington, MA) and frozen at −80° C.

Thawed clarified culture medium was loaded onto a MabSelect™ SuRe™ Protein-A column (GE Healthcare, Chicago, IL) and washed with 10 column volumes of PBS buffer at pH 7.2. The antibody was eluted with 10 column volumes of citrate buffer at pH 3.6 with the pooled fractions containing the antibody neutralized with TRIS at pH 11.

The antibody-containing protein-A eluate was further purified by size exclusion chromatography (SEC). For SEC, samples were loaded onto a Sephadex 200 HiLoad® 16/60 200 prep grade column (GE Healthcare, Chicago, IL) using an AKTA™ purification system (GE Healthcare, Chicago, IL; Express, FPLC or Purifier system) at a flow-rate of 1 mL/min. PBS buffer at pH 7.4 was used at a flow-rate of 1 mL/min. Fractions corresponding to the purified antibody were pooled based on SDS-PAGE or capillary electrophoresis analysis (LabChip GX®; PerkinElmer, Inc., Waltham, MA) and if necessary concentrated to 5-10 mg/mL. In addition, if low endotoxin was a requirement for downstream analytics, then systems, columns and resin (where applicable) were depyrogenated using NaOH solutions with standard protocols prior to protein purification.

### 2. Drug Conjugation

Cysteine engineered variants and controls were expressed in cysteine capped form with an L-cysteine cap, a glutathione cap, or a combination of both. To reduce sample heterogeneity and increase conjugation efficiency, all antibodies were subjected to a reduction-oxidation step prior to conjugation with the maleimide activated drug-linkers. A representative procedure is provided below.

3 mg of a 5 mg/mL solution of variant v28983 (see Table 2.2; MW 146301 Da) was reduced in PBS, pH 7.4 with 25 eq. molar excess of tris(2-carboxyethyl) phosphine (TCEP) for 3 hrs at 37° C. (water bath) in the presence of 1 mM diethylenetriaminepentaacetic acid (DTPA) based on the following calculation (Table A):

TABLE A
Sample Reduction of Cysteine Insertion Variant, v28983

| Antibody | v28983 |
|---|---|
| Target SH/mAb | 2 |
| mAb MW (Da) | 146301 |
| mAb Conc, mg/mL | 5.00 |
| mAb Conc, M | 3.42E−05 |
| amt mAb, mg | 3 |
| mAb, μl | 600.0 |
| TCEP eq | 25 |
| 10 mM TCEP, μl | 51.3 |
| 5 mM DTPA in PBS pH 7.4, μl | 170.0 |
| DTPA Final Conc, mM | 1 |
| PBS, μl | 28.7 |
| Total vol, μl | 850.0 |
| Final conc, mg/mL | 3.5 |

After reduction was complete, the excess TCEP was removed using a 5 mL 40 kD Zeba™ Spin Desalting Column (Thermo Fisher Scientific, Waltham, MA) equilibrated with PBS, pH 7.4. The reduced antibody was subjected to overnight oxidation (18 hrs) with 25 molar excess dehydroascorbic acid (DHAA) (assuming 100% recovery from the Zeba™ column purification) at 4° C. to re-form the interchain disulphide bonds while keeping the inserted cysteine in reduced (free thiol) form. Addition of DHAA was based on the following calculation (Table B):

TABLE B
Sample Oxidation of Cysteine Insertion Variant, v28983

| Antibody | v28983 |
|---|---|
| Target SH/mAb | 2 |
| mAb MW (Da) | 146301 |
| mAb Conc, mg/mL | 5.00 |
| mAb Conc, M | 3.42E−05 |
| amt mAb, mg | 3 |
| DHAA eq | 25 |
| 15 mM DHAA, μl | 51.3 |
| DHAA mM | 10.0 |

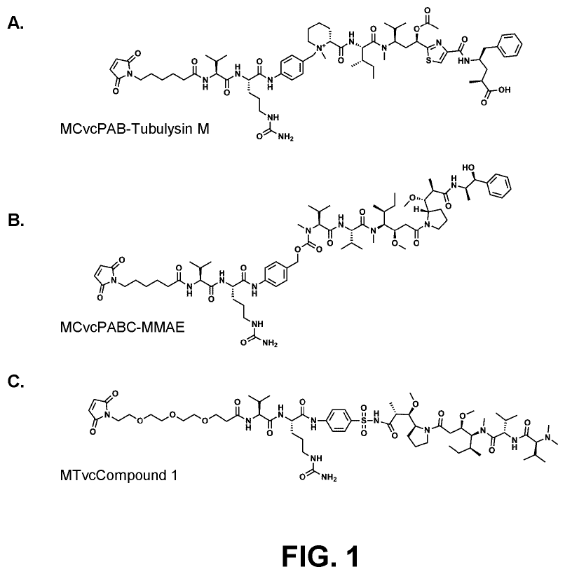

The oxidized antibody was divided into three aliquots of 1 mg for conjugation to three different drug linkers: MTvcCompound 1, MCvcPABC-MMAE and MCvcPAB-Tubulysin M. The structures of the three drug-linkers are shown in FIG. 1. Conjugation was achieved by incubation with 5 molar excess of drug-linker at room temperature. Drug-linkers were prepared as 10 or 20 mM DMSO stocks and added to the reaction based on following calculation (Table C):

TABLE C
Conjugation of Cysteine Insertion Variant,
v28983 with Three Different Drug-Linkers

| Antibody | v28983 | v28983 | v28983 |
|---|---|---|---|
| Target SH/mAb | 2 | 2 | 2 |
| Drug-Linker | MTvcCom- | MCvcPABC- | MCvcPAB- |
| | pound 1 | MMAE | Tubulysin M |
| Drug-Linker Conc, mM | 20 | 10 | 10 |
| Drug-Linker eq | 5 | 5 | 5 |
| mAb, mg | 1 | 1 | 1 |
| mAb MW (Da) | 146301 | 146301 | 146301 |
| Drug-Linker Volume, ul | 1.7 | 3.5 | 3.5 |

### 3. Differential Scanning Calorimetry (DSC)

The thermal stability of cysteine engineered antibodies was measured using DSC as follows. 400 μL of purified sample at concentrations of either 0.2 mg/ml or 0.4 mg/mL in PBS were used for DSC analysis with a MicroCal VP-Capillary DSC™ (GE Healthcare, Chicago, IL). At the start of each DSC run, 5 buffer blank injections were performed to stabilize the baseline, and a buffer injection was placed before each sample injection for referencing. Each sample was scanned from 20 to 100° C. at a 60° C./hr rate, with low feedback, 8 sec filter, 5 min preTstat, and 70 psi nitrogen pressure. The resulting thermograms were referenced and analyzed using Origin 7 software (OriginLab Corporation, Northampton, MA).

### 4. Hydrophobic Interaction Chromatography (HIC)

For HIC runs, TSKgel Butyl-NPR (2.5 μm, 4.6×35 mm) column (TOSOH Bioscience GmbH, Griesheim, Germany) was equilibrated with 5 column volumes of Buffer A (1.5 M (NH₄)₂SO₄, 25 mM PO4³⁻, pH 6.95) at room temperature. Typically, 20-30 ug of sample at 2-3 mg/mL concentration was loaded on the column with 95% Buffer A and 5% Buffer B (75% 25 mM PO₄³⁻ pH 6.95, plus 25% isopropanol) and run for 15 mins at 0.5 mL/min using the following gradient (Table D):

TABLE D
HIC Gradient

| Time (min) | % Buffer A | % Buffer B |
|---|---|---|
| 0 | 95 | 5 |
| 0.1 | 95 | 5 |
| 5 | 80 | 20 |
| 9.5 | 65 | 35 |
| 11.5 | 50 | 50 |
| 12.5 | 5 | 95 |
| 13.5 | 5 | 95 |
| 12.6 | 95 | 5 |
| 15 | 95 | 5 |

For each sample, the HIC chromatogram was integrated using appropriate parameters that provided complete, baseline-to-baseline integration of each peak, followed by integration of each peak showing reasonable separations. The peaks corresponding to the distinct DAR species within the samples were identified. The DAR 0 peak exhibited consistent retention time with the naked (reduced-oxidized) antibody. For an ADC comprising a single cysteine insertion variant, each subsequent peak represents DAR 1 and DAR 2.

The DAR by HIC was calculated based on the AUC for individual DAR species (0, 1 and 2):

$$Calculated DAR = ( \% AUCx 0 + \% AUCx 1 + \% AUCx 2 ) / 100$$

The HIC retention time (HIC-RRT) for individual ADCs were calculated as follows:

$$HIC - RRT = RT of Target DAR / RT of DAR 0$$

To minimize the effect of cysteine capping of antibodies on HIC-RRT, RT of DARO refers to the retention time of reduced-oxidized antibody without conjugation to payload. Each variant has its own DARO RT for the HIC-RRT calculation.

### 5. Analytical Size Exclusion Chromatography (SEC)

For analytical SEC runs, an Agilent Advance Bio SEC column (300 Å, 2.7 μm, 7.8×150 mm) (Agilent Technologies, Inc., Santa Clara, CA; serial #6377910-24) was equilibrated with 5 column volumes of Buffer A (150 mM NaₓPO₄, pH 6.95) at room temperature. Typically, 20-30 ug of sample at 2-3 mg/mL concentration was loaded onto the column and run for 7 mins at 1 mL/min in an isocratic manner and absorbance at 280 nm was reported. For each sample, the chromatogram was integrated to provide complete, baseline-to-baseline integration of each peak, with reasonably placed separation between partially resolved peaks. The peak corresponding to the major component for IgG (approximate retention time 3.3 min) was reported as the monomer based on the SEC profile of the control IgGI antibody, trastuzumab. Any peak occurring prior to 3.3 min was designated as HMWS, and any peak occurring after 3.3 min was designated as LMWS, excluding solvent peaks (over 5.2 min).

### 6. Liquid Chromatography-Mass Spectrometry (LC-MS)

For DAR by LC-MS, ADCs were diluted to 1 mg/mL in PBS, pH 7.4, then deglycosylated. For deglycosylation, typically lug EndoS was employed for every 10 ug ADC and the reaction mix was incubated at room temperature for an hour. Samples were reduced by adding 3 uL 500 mM TCEP to each 10 uL sample followed by incubation at 70° C. for an hour. Finally, samples were run on LC-MS quadrupole time-of-flight (QTOF) system (Agilent 1290 HPLC connected to Agilent 6545 QTOF; Agilent Technologies, Inc., Santa Clara, CA), 1 uL injection each. The detailed procedure is described below.
Column: PLRP-S 1000 Å, 8 uM, 50×2.1 mm (Agilent Technologies, Inc., Santa Clara, CA)
Mobile phase C: 0.1% formic acid, 0.025% trifluoroacetic acid and 10% isopropyl alcohol in H₂O
Mobile Phase D: 0.1% formic acid and 10% isopropyl alcohol in acetonitrile
Detection: signal A (280 nm, 4.0 band width), signal B (220 nm, 4.0 band width)
Gradient:

| Time (Min) | Buffer C | Buffer D |
|---|---|---|
| 0 | 80 | 20 |
| 20 | 60 | 40 |
| 22 | 10 | 90 |
| 22.5 | 1 | 99 |
| 24 | 1 | 99 |

Post Run Time: 2 minutes

### 7. Capillary Electrophoresis-SDS (CE-SDS)

Initially, all samples were diluted to 1 mg/mL before preparing the samples in a 96-well PCR plate following manufacturer's protocol (Protein Express Assay LabChip™; PerkinElmer, Inc., Waltham, MA). Briefly, 2 uL ADC was mixed with 7 uL Protein Express buffer in the presence or absence of 400 mM dithiothreitol (DTT) as reducing agent, followed by heat denaturation at 95° C. for 5 minutes. Samples were then diluted in dH₂O at a 1:2 ratio before data acquisition. After each CE-SDS run, the gel and corresponding electropherogram was analyzed using LabChip™ Reviewer (PerkinElmer, Inc., Waltham, MA).

### Example 1: Identification of Potential Cysteine Insertion Sites by In Silico ENGINEERING

Identification of putative cysteine insertion sites in IgGI were carried out on explicit-solvent molecular dynamics (MD) trajectories for a model Fab (derived from PDB ID 1JPT, D3H44) and model Fc molecule (derived from PDB ID 1E4K) according to the following guiding criteria:
Exclusion of CDRs
Avoidance of secondary structures
Relative solvent accessible surface area (SASA): rSASA>30%
Avoidance of interference with protein A and FcRn binding

Positions were then labelled according to rSASA and root mean square fluctuation (RMSF) as follows:
Type 1: desirable rSASA (30-60%) and mobile (RMSF above mean+1 standard deviation (SD) threshold)
Type 2: desirable rSASA (30-60%) but ordered (RMSF below mean+1SD threshold)
Type 3: exposed (rSASA>60%) and mobile (RMSF above mean+1SD threshold)
Type 4: exposed (rSASA>60%) but ordered (RMSF below mean+1SD threshold)

Cysteine insertion sites (“designs”) in Phase 1 were proposed based on a structure-guided, semi-rational approach. Putative insertion sites were ranked based on their risk (interference with other known ligands and disulphide scrambling), environment (the Type 1-4 label as described above) and presumed likelihood of structural impact and conjugation stability. A total of 13 designs were proposed in Phase I that sampled different structural regions of IgG1.

Phase 2 involved conducting modelling experiments for each individual putative cysteine insertion and selection of variants based on parameters calculated from the implicit-solvent molecular dynamics (MD) trajectories for the modelled insertions. Briefly, each loop (n residues) of interest was removed and an n+1 loop from a deposited structure in the RCSB PDB (Research Collaboratory for Structural Bioinformatics-Protein Data Bank) was grafted in its place based on lowest root mean square deviation (RMSD) with the anchoring boundary residues. Each loop was then mutated to match the original sequence with a cysteine residue inserted at each relevant position. For each grafted-loop model, an implicit-solvent MD trajectory was calculated and designs were ranked according to parameters and criteria described in Table 1.1 below.

For the few cases in which the loop-grafting algorithm failed to provide a solution, designs from those loops were selected using the methodology described for the Phase 1 designs. In total 32 variants were generated by this process, 13 variants in Phase 1 and 19 variants in Phase 2.

TABLE 1.1
Parameters and Criteria for Ranking Designs

| Metric | Expected Range or Behaviour | Correlates With | Relevance |
|---|---|---|---|
| CYS pKa | >9.5 preferred | Conjugate | High |
| | 9.0-9.5 acceptable | stability | |
| Visual | Anchor residue positions | Protein stability | High |
| Inspection | Graft conformation confidence | Metrics confidence | |
| | Likelihood of affecting | | |
| | secondary structures | | |
| | Exposure/burial of polar/apolar | | |
| | residues | | |
| CYS SASA | rSASA: optimum range ~10-50% | Conjugation | Medium |
| | SASA: <60 Å | efficiency | |
| | | Conjugate stability | |
| Loop per | Residues in the middle of the loop | Protein stability | Medium |
| residue RMSF | more mobile than residues close to | Conjugation | |
| | the loop anchors, as judged by | efficiency | |
| | RMSF. | | |
| | RMSF <1: very rigid | | |
| Amber_folding | Internal loop comparison. | Protein | Low |
| | Lower value: more stable | stability | |
| Average Total | Overall negative environment | Conjugate | Low |
| Charge | purportedly preferable | stability | |
| Presence | Presence of +ve charge close to | | |
| of +ve residue | CYS-linkage purportedly beneficial | | |

### EXAMPLE 2: In Vitro CHARACTERIZATION OF INITIAL VARIANTS

The 32 variants from Example 1, together with the controls shown in Table 2.1, were cloned and expressed as described in General Procedure 1. Two additional constructs, v27321 and v27322, were also included in this initial characterization. These two variants include a cysteine insertion before and after position K149, respectively. A cysteine substitution at position K149 has previously been described (Vollmar, et al., 2017, Bioconjug Chem, 28 (10): 2538-2548).

Throughout the Examples, the following nomenclature is used for the cysteine insertion mutations. Position numberings are Kabat for the Fab region (VH, VL, CHI, and CL) and EU for the Fc region (CH2 and CH3). All cysteine insertions are numbered based the residue preceding the insertion with reference to the unmodified Heavy Chain (H) or Light Chain (L) followed by “0.5”. For example, L_K149.5C indicates a Cysteine inserted after the residue Lys 149 in the Light Chain.

TABLE 2.1
Controls and Additional Variants for
Initial in vitro Characterization

| | Insertion/ | | |
|---|---|---|---|
| Variant | Substitution¹ | Domain | Description |
| v22758 | H_A114C | Fab/CH1 | Control (THIOMAB ™) |
| | | | Cysteine substitution² |
| v29008 | H_S239C | Fc/CH2 | Control |
| | | | Cysteine substitution³ |
| v29013 | H_S239.5C | Fc/CH2 | Control |
| | | | Cysteine insertion |
| v27320 | L_K149C | Fab/CL | Control |
| | | | Cysteine substitution⁴ |
| v27323 | H_A138C | Fab/CH1 | Control |
| | | | Cysteine substitution |
| v27321 | L_W148.5C | Fab/CL | Cysteine insertion |
| v27322 | L_K149.5C | Fab/CL | Cysteine insertion |

¹H = heavy chain; L = light chain
²Junutula, et al., 2008, Nature Biotechnology, 26: 925-932
³Sussman, et al., 2018, Protein Engineering, Design and Selection, 31(2): 47-54
⁴Vollmar, et al., 2017, Bioconjug Chem, 28(10): 2538-2548

Each antibody was then conjugated to MTvcCompound 1 or MCvcPABC-MMAE as described in General Procedure 2. Preliminary in vitro characterization of each antibody and ADC was conducted as described in General Procedures 3-6. The variants were ranked based on the following criteria:
DSC: Tm difference from parental antibody defined as
   No Change (0° C.≤Tm difference≤3° C.)
   Small (3° C.<Tm difference≤8° C.)
   High (Tm difference>8° C.) high
Yield of antibody from a single 500 mL transfection in CHO cells
DAR (MS) drug-antibody ratio determined by LC-MS
DAR (HIC) drug-antibody ratio determined by HIC
Relative retention time (RRT D2/DO) calculated by dividing the HIC retention time of the DAR 2 species by the HIC retention time of the DAR 0 species

The 10 variants that showed the most favourable characteristics were selected for further characterization. The properties determined from the preliminary characterization of these 10 variants and the 5 controls are shown in Table 2.2.

TABLE 2.2
Preliminary Characterization of Variants and ADCs

| | | | | | | | MCvcPABC-MMAE | | | MTvcCompound 1 | | |
|---|---|---|---|---|---|---|---|---|---|---|---|---|
| | Insertion/ | | | | ΔTm (by | Yield | DAR | DAR | RRT | DAR | DAR | RRT |
| Variant | Substitution | Phase | Domain | Loop | DSC) | (mg) | (MS) | (HIC) | (D2/D0) | (MS) | (HIC) | (D2/D0) |
| v22758 | H_A114C | Control | Fab/ | — | No Change | 6 | 1.7 | 1.6 | 1.36 | 1.7 | 1.7 | 1.1 |
| | | | CH1 | | | | | | | | | |
| v29008 | H_S239C | Control | Fc/CH2 | — | No Change | 5.32 | 1.5 | 1.2 | 1.29 | 1.7 | 1.7 | 1.08 |
| v29013 | H_S239.5C | Control | Fc/CH2 | — | High | 10.91 | 1.9 | 1.9 | 1.2 | 1.8 | 2 | 1.01 |
| v27320 | L_K149C | Control | Fab/CL | — | No Change | 4.29 | 0.9 | 1 | 1.58 | 1.3 | 1 | 1.14 |
| v27323 | H_A138C | Control | Fab/ | — | No Change | 8.84 | 0.9 | 0.9 | 1.36 | 0.8 | 1.0 | 1.09 |
| | | | CH1 | | | | | | | | | |
| v22760¹ | L_K39.5C | 1 | Fab/VL | L3 | Small | 1.6 | 2 | 1.6 | 1.16 | 1.9 | 1.8 | 1.04 |
| v22761¹ | L_K126.5C | 1 | Fab/CL | L8 | No Change | 2.8 | 1.6 | 2 | 1.36 | 1.4 | 1.8 | 1.08 |
| v22765 | H_G237.5C | 1 | Fc/CH2 | FC1 | No Change | 11.5 | 1.8 | 1.8 | 1.35 | 1.8 | 1.7 | 1.08 |
| v22768 | H_Q295.5C | 1 | Fc/CH2 | FC6 | No Change | 2.3 | 1.9 | 1.5 | 1.36 | 1.7 | 1.5 | 1.12 |
| v28983 | L_P40.5C | 2 | Fab/VL | L3 | No Change | 2.54 | 2 | 1.8 | 1.17 | 2 | 1.8 | 1.04 |
| v28989 | H_A9.5C | 2 | Fab/VH | H1 | No Change | 6.48 | 1.8 | 1.9 | 1.16 | 1.9 | 2 | 1.06 |
| v28993 | H_G169.5C | 2 | Fab/ | H9a | No Change | 13.75 | 1.9 | 1.8 | 1.48 | 1.9 | 1.9 | 1.14 |
| | | | CH1 | | | | | | | | | |
| v29001 | H_T299.5C | 2 | Fc/CH2 | FC6 | Small | 5.76 | 2 | 2 | 1.05 | 2 | 2 | 1.02 |
| v27322 | L_K149.5C | 2 | Fab/CL | — | No Change | 0.94 | 1.9 | 1.8 | 1.41 | 2 | 1.8 | 1.08 |
| v27321² | L_W148.5C | 2 | Fab/CL | — | ND³ | ND | ND | ND | ND | ND | ND | ND |

¹Variant included the peptide ESSCDVKLV [SEQ ID NO: 2] fused to the C-terminal residue of the light chain
²v27321 had limited expression
³ND = not determined

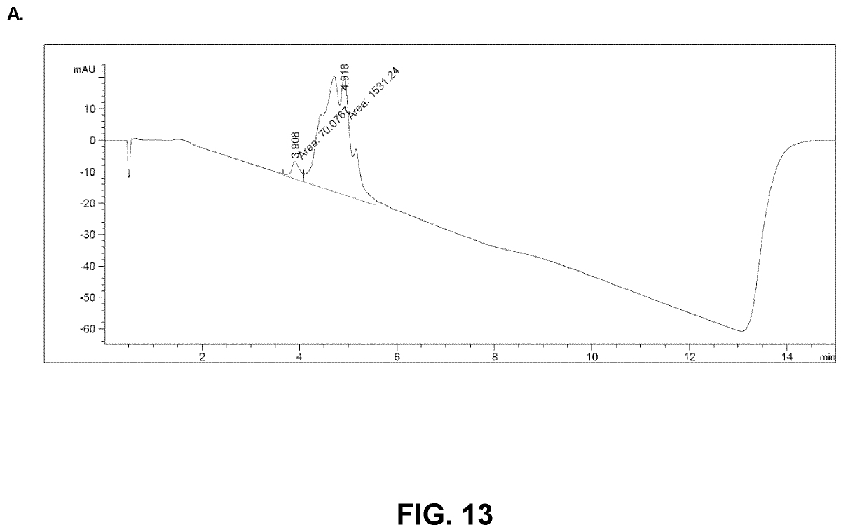

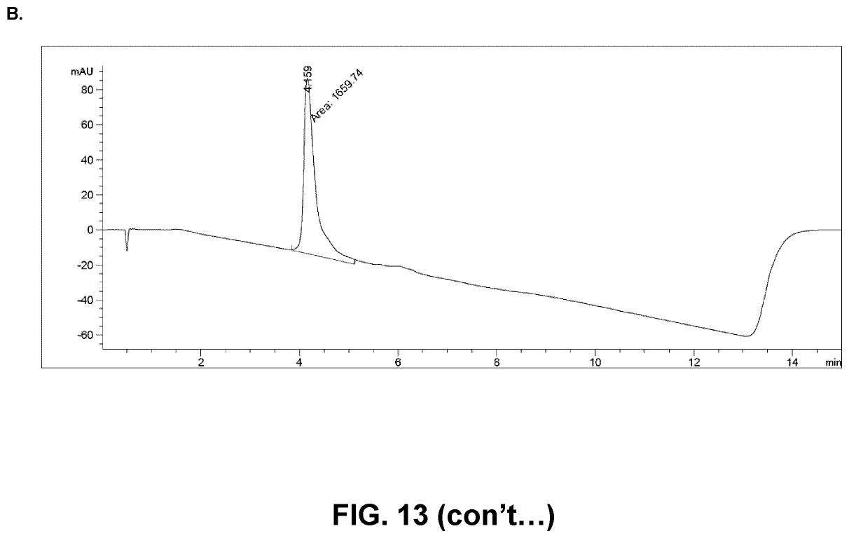

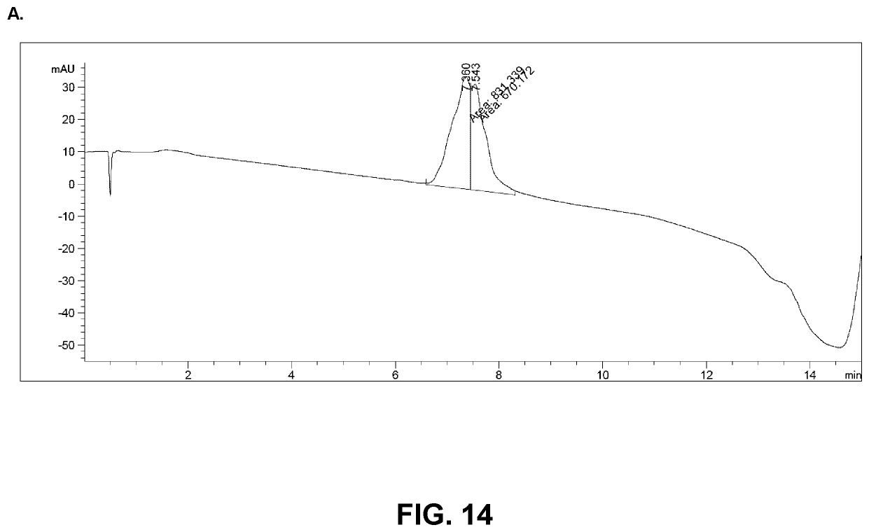

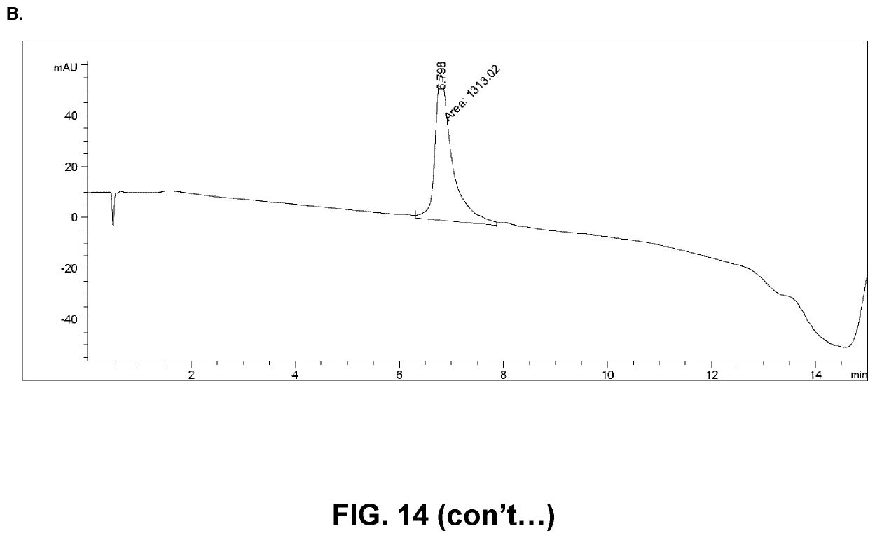

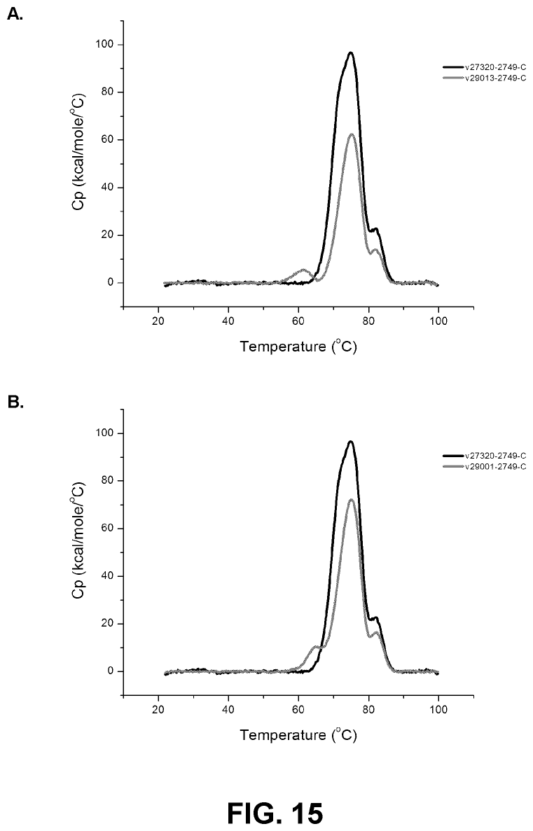

Examples of HIC profiles for one variant (v29001 (H_T299.5C)) and a control variant (v29013 (H_S239.5C)) conjugated to MCvcPABC-MMAE or MTvcCompound 1 are shown in FIGS. 13 and 14, respectively. The DSC profiles for the same two variants (unconjugated) are shown in FIG. 15.

The HIC profiles for control variant v29013 (H_S239.5C) can be seen to consist of multiple peaks indicating the presence of multiple species (FIGS. 13A and 14A), whereas the HIC profiles for variant v29001 (H_T299.5C) show a single monomeric peak (FIGS. 13B and 14B). FIG. 14 also shows that the MTvcCompound 1 conjugate generated with variant v29001 appeared less hydrophobic (lower HIC-RRT) than the MTvcCompound 1 conjugate generated with control variant v29013. The DSC profiles in FIG. 15 show a more pronounced destabilization was observed for control variant v29013 (Tm for the CH2 domain of 62° C.) than for variant v29001 (Tm for the CH2 domain of 65° C.). The Tm for the CH3 domain for both these variants was very similar, confirming the cysteine insertion has no effect on the stability of this domain.

### Example 3: Preparation of Antibody-Drug Conjugates Comprising Cysteine Insertion Variants

Each of the 10 variants shown in Table 2.2 together with two of the controls, v22758 (H_A114C) and v29013 (H_S239.5C), were conjugated to three different drug-linkers (MTvcCompound 1, MCvcPABC-MMAE and MCvcPAB-Tubulysin M; see FIG. 1) following General Procedure 2.

The resulting 36 ADCs were characterized by hydrophobic interaction chromatography (HIC), size-exclusion chromatography (SEC), liquid chromatography-mass spectrometry (LC-MS), capillary electrophoresis SDA (CE-SDS) and an on-cell binding assay as described in Examples 4-8.

### Example 4: In Vitro Characterization-Hydrophobic Interaction Chromatography

The ADCs from Example 3 were characterized by hydrophobic interaction chromatography (HIC) as described in General Procedure 4. HIC allows for separation of different proteins based on their inherent hydrophobicity and because it is a non-denaturing method, HIC allows for separation of the different DAR species comprised by a given ADC. HIC is also a useful technique to rank ADCs based on their relative retention time (RRT). As hydrophobic ADCs may clear more quickly from circulation in vivo, HIC-RRT values are a potentially valuable biophysical parameter to identify the most useful cysteine insertion sites.

HIC was employed to determine the DAR for all ADCs conjugated to MTvcCompound 1, MCvcPABC-MMAE and MCvcPAB-Tubulysin M, and the HIC-RRT for all ADCs conjugated to MTvcCompound 1 and MCvcPABC-MMAE. The results are shown in Tables 4.1 and 4.2.

TABLE 4.1
DAR Values as Determined by HIC

| | Insertion/ | Drug-Linker | | |
|---|---|---|---|---|
| | Substitution | MTvcCom- | MCvcPABC- | MCvcPAB- |
| Variant | Position | pound 1 | MMAE | Tubulysin M |
| v22760¹ | L_K39.5C | 1.8 | 1.7 | 1.7 |
| v22761¹ | L_K126.5 | 1.7 | 1.7 | 1.7 |
| v22765 | H_G237.5C | 1.5 | 1.7 | 1.5 |
| v22768 | H_Q295.5C | 1.5 | 1.4 | 1.4 |
| v27321 | L_W148.5C | 1.7 | 1.6 | 1.7 |
| v27322 | L_K149.5C | 1.6 | 1.7 | 1.7 |
| v28983 | L_P40.5C | 1.8 | 1.7 | 1.8 |
| v28989 | H_A9.5C | 2.0 | 1.8 | 1.9 |
| v28993 | H_G169.5C | 1.7 | 1.8 | 1.8 |
| v29001 | H_T299.5C | 2.0 | 2.0 | 2.0 |
| v22758 | H_A114C | 1.7 | 1.6 | 1.7 |
| (Control) | | | | |
| v29013 | H_S239.5C | 2.0 | 1.9 | 1.9 |
| (Control) | | | | |

¹Variant included the peptide ESSCDVKLV [SEQ ID NO: 2] fused to the C-terminal residue of the light chain

TABLE 4.2
HIC-RRT Values

| | Insertion/ | Drug-Linker | |
|---|---|---|---|
| | Substitution | | MCvcPABC- |
| Variant | Position | MTvcCompound 1 | MMAE |
| v22760¹ | L_K39.5C | 1.09 | 1.16 |
| v22761¹ | L_K126.5 | 1.12 | 1.36 |
| v22765 | H_G237.5C | 1.14 | 1.35 |
| v22768 | H_Q295.5C | 1.18 | 1.36 |
| v27321 | L_W148.5C | 1.15 | ND² |
| v27322 | L_K149.5C | 1.15 | 1.41 |
| v28983 | L_P40.5C | 1.09 | 1.18 |
| v28989 | H_A9.5C | 1.10 | 1.16 |
| v28993 | H_G169.5C | 1.19 | 1.48 |
| v29001 | H_T299.5C | 1.03 | 1.05 |
| v22758 | H_A114C | 1.15 | 1.36 |
| (Control) | | | |
| v29013 | H_S239.5C | 1.08 | ND |
| (Control) | | | |

¹Variant included the peptide ESSCDVKLV [SEQ ID NO: 2] fused to the C-terminal residue of the light chain
²ND = Not determined due to poor separation of DAR species

Overall, all the ADCs showed a DAR range between 1.4 and 2.0. One variant, v29001, when conjugated to each of the three drug-linkers showed a single peak on HIC with complete loading of DAR 2.0 in each case. Variant v22768 showed the lowest drug loading, only 1.4-1.5 with each drug-linker. Conjugation efficiency was expected to be site-dependent due to the impact of thiol pKa, SASA and local environment of the inserted cysteine and this was reflected in the DAR values (Table 4.1).

The drug-linker MTvcCompound 1 is relatively hydrophilic. Conjugation of this drug-linker to any of the site-specific cysteine insertion variants had minimal effect on the HIC retention time. For most of the variants conjugated to MTvcCompound 1, the HIC-RRT values were below 1.15, which was also the observed HIC-RRT value for the control v22758 ADC conjugated to the same drug-linker. Two variants, v22768 and v28993, conjugated to MTvcCompound 1 showed slightly higher HIC-RRT values than the control v22758 conjugated to the same drug-linker: 1.18 and 1.19, respectively. Variant v29001 conjugated to MTvcCompound 1 appeared to be less hydrophobic than both the controls (v22758 and v29013) conjugated to the same drug-linker.

The drug-linker MCvcPABC-MMAE is more hydrophobic than MTvcCompound 1 and hence the HIC-RRT values for all ADCs comprising MCvcPABC-MMAE were higher than the respective MTvcCompound 1 conjugates. Overall, a similar trend was observed for MCvcPABC-MMAE conjugates as was observed for MCvcCompound 1 conjugates: v28993 conjugated to MCvcPABC-MMAE showed the highest HIC-RRT value, and v29001 conjugated to MCvcPABC-MMAE showed the lowest HIC-RRT value (1.05). For one variant, v27321 conjugated to MCvcPABC-MMAE, as well as the control v29013 conjugated to the same drug-linker, the HIC-RRT values could not be determined due to poor resolution of the HIC profiles for each of these two ADCs.

### Example 5: In vitro Characterization—Size Exclusion Chromatography

The ADCs from Example 3 were further characterized by size exclusion chromatography (SEC). SEC is a useful technique for estimating the size of proteins and determining the presence of aggregation/high molecular weight species (HMWS) and fragmentation/low molecular weight species (LMWS) in a protein preparation.

During preparation of the ADCs, any improper oxidation resulting from disulphide bond formation through the inserted cysteine residues could lead to formation of concatemers and other HMWS. To investigate the molecular size and relative abundance of different species, each of the ADCs was analyzed by SEC as described in General Procedure 5.

The results are shown in Table 5.1.

TABLE 5.1
Percent of Monomer, HMWS and LMWS in ADC Preparations as Determined by HPLC-SEC

| | | Drug-Linker | | | | | | | | |
|---|---|---|---|---|---|---|---|---|---|---|
| | Insertion/ | MTvcCompound 1 | | | MCvcPABC-MMAE | | | MCvcPAB-Tubulysin M | | |
| | Substitution | Monomer | HMWS | LMWS | Monomer | HMWS | LMWS | Monomer | HMWS | LMWS |
| Variant | Position | % | % | % | % | % | % | % | % | % |
| v22760¹ | L_K39.5C | 99 | 1 | 0 | 98 | 1 | 1 | 97 | 1 | 2 |
| v22761¹ | L_K126.5 | 99 | 1 | 0 | 99 | 1 | 0 | 97 | 1 | 1 |
| v22765 | H_G237.5C | 98 | 1 | 1 | 97 | 1 | 2 | 97 | 1 | 2 |
| v22768 | H_Q295.5C | 94 | 6 | 0 | 97 | 3 | 0 | 97 | 3 | 0 |
| v27321 | L_W148.5C | 99 | 1 | 0 | 99 | 1 | 0 | 99 | 1 | 0 |
| v27322 | L_K149.5C | 94 | 3 | 2 | 96 | 2 | 2 | 93 | 2 | 5 |
| v28983 | L_P40.5C | 98 | 1 | 1 | 98 | 1 | 1 | 97 | 1 | 2 |
| v28989 | H_A9.5C | 99 | 1 | 0 | 98 | 1 | 1 | 99 | 1 | 0 |
| v28993 | H_G169.5C | 96 | 1 | 3 | 96 | 1 | 3 | 94 | 2 | 4 |
| v29001 | H_T299.5C | 99 | 1 | 0 | 97 | 1 | 3 | 92 | 1 | 7 |
| v22758 | H_A114C | 95 | 1 | 4 | 97 | 1 | 2 | 97 | 1 | 2 |
| (Control) | | | | | | | | | | |
| v29013 | H_S239.5C | 99 | 1 | 0 | 97 | 1 | 1 | 99 | 1 | 0 |
| (Control) | | | | | | | | | | |

¹Variant included the peptide ESSCDVKLV [SEQ ID NO: 2] fused to the C-terminal residue of the light chain

As can be seen from Table 5.1, all ADC preparations contained >90% monomer by HPLC-SEC. In general, the ADCs comprising the drug-linker MTvcCompound 1 showed the highest monomer content with the exception of variant v22768, which showed highest HMWS content (6%) and lowest monomer content (94%) for the MTvcCompound 1 conjugates. The control ADC, v22758 (A114C) conjugated to MTvcCompound 1, showed a monomer content of 95%.

ADCs comprising the MCvcPABC-MMAE drug-linker showed a similar trend to those comprising MTvcCompound 1, with a monomer range between 97% and 99%, with low amounts of HMWS and LMWS.

In contrast, ADCs comprising the MCvcPAB-Tubulysin M drug-linker showed lower monomer content than ADCs comprising either of the other two drug-linkers. The ADC, v29001-MCvcPAB-Tubulysin M, showed the lowest monomer content (92%) and highest LMWS content (7%) of all the ADCs tested.

### Example 6: In vitro Characterization—Liquid Chromatography-Mass Spectrometry (LC-MS)

The ADCs from Example 3 were further characterized by liquid chromatography-mass spectrometry (LC-MS) as described in General Procedure 6.

LC-MS is a standard analytical method for measuring the drug-antibody ratio (DAR) and the drug load distribution of ADCs. A general procedure for LC-MS based DAR measurement at intact ADC level involves deconvoluting the mass spectra to a series of “zero-charge” masses, and then obtaining DAR distribution or computing average DAR by integrating and weighting the spectral peak area or peak intensities.

For analysis of the ADCs from Example 3, ADCs were deglycosylated prior to LC-MS analysis by treatment with EndoS, which removed all the attached carbohydrate moieties apart from the reducing terminal N-acetyl glucosamine (GlcNAc) and fucose (Fuc). Some of the ADCs were also treated with IdeS, a protease which cleaves directly after the hinge cysteine at position ²³⁶G-G²³⁷ of the heavy chain. Reduction of IdeS-treated samples yielded three different species: Fc/2, Fd and LC. DAR determination by MS for IdeS-treated samples further improved DAR measurement accuracy and provided supplementary structural information of ADCs.

The average DAR as determined by LC-MS for each of the ADCs is shown in Table 6.1.

TABLE 6.1
DAR Values as Determined by LC-MS

| | Insertion/ | Drug-Linker | | |
|---|---|---|---|---|
| | Substitution | MTvcCom- | MCvcPABC- | MCvcPAB- |
| Variant | Position | pound 1 | MMAE | Tubulysin M |
| v22760¹ | L_K39.5C | 1.9 | 2.0 | 2.0 |
| v22761¹ | L_K126.5 | 1.8 | 1.8 | 1.8 |
| v22765 | H_G237.5C | 1.7 | 1.8 | 1.8 |
| v22768 | H_Q295.5C | 2.0 | 2.0 | 1.9 |
| v27321 | L_W148.5C | 1.8 | 1.8 | 1.8 |
| v27322 | L_K149.5C | 1.8 | 1.9 | 1.9 |
| v28983 | L_P40.5C | 2.0 | 2.0 | 2.0 |
| v28989 | H_A9.5C | 1.9 | 1.9 | 1.9 |
| v28993 | H_G169.5C | 1.9 | 1.9 | 1.9 |
| v29001 | H_T299.5C | 2.0 | 2.0 | 2.0 |
| v22758 | H_A114C | 1.8 | 1.7 | 1.7 |
| (Control) | | | | |
| v29013 | H_S239.5C | 2.0 | 2.0 | 1.9 |
| (Control) | | | | |

¹Variant included the peptide ESSCDVKLV [SEQ ID NO: 2] fused to the C-terminal residue of the light chain

As shown in Table 6.1, the DAR for the ADCs as determined by LC-MS ranged between 1.7 and 2.0, indicating that all three drug-linkers had similar conjugation efficiencies. No detectable conjugation was observed for hinge or interchain disulphide cysteine residues.

Overall, the DAR determined by LC-MS correlated well with the DAR obtained by HIC (see Table 4.1). For example, variant v29001 when conjugated to each of the three drug-linkers showed DAR 2 by both LC-MS and HIC measurement. For variants v27321 and v29013 conjugated to MCvcPABC-MMAE, calculation of DAR was unsuccessful by HIC due to poor separation of the relevant peaks. However, DAR for these two ADCs was determined successfully by LC-MS (DAR 1.8 and 2.0, respectively).

### Example 7: In vitro Characterization—Capillary Electrophoresis-SDS (CE-SDS)

Each of the ADCs from Example 3 was assessed by capillary electrophoresis-SDS (CE-SDS) under reducing and non-reducing conditions as described in General Procedure 7 in order to evaluate the purity of the samples.

Under reducing conditions, the inter-chain disulphide bonds in the ADC antibody are reduced to yield the corresponding heavy chains (HC) and light chains (LC), which can be separated by the difference in their molecular weights. In contrast, under non-reducing conditions, the antibody remains intact and can be separated from any partial antibodies or antibody fragments, as well as any concatemers. The intact full length IgG1 (2H-2L) has the highest molecular weight of approximately 150 kDa, followed by 2H-L, HH, HL, H, L fragments with molecular weights of approximately 125, 100, 75, 50 and 25 kDa, respectively. An incomplete or partial oxidation of the antibody during the preparation of the ADC will lead to the presence of some or all of these LMWS in the sample, whereas over-oxidation will lead to cysteine-cysteine concatenated oligomeric HMWS.

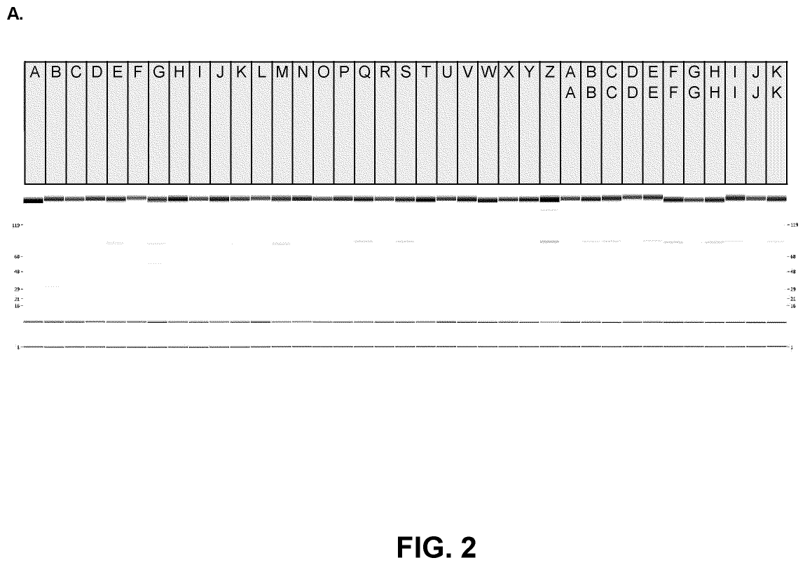

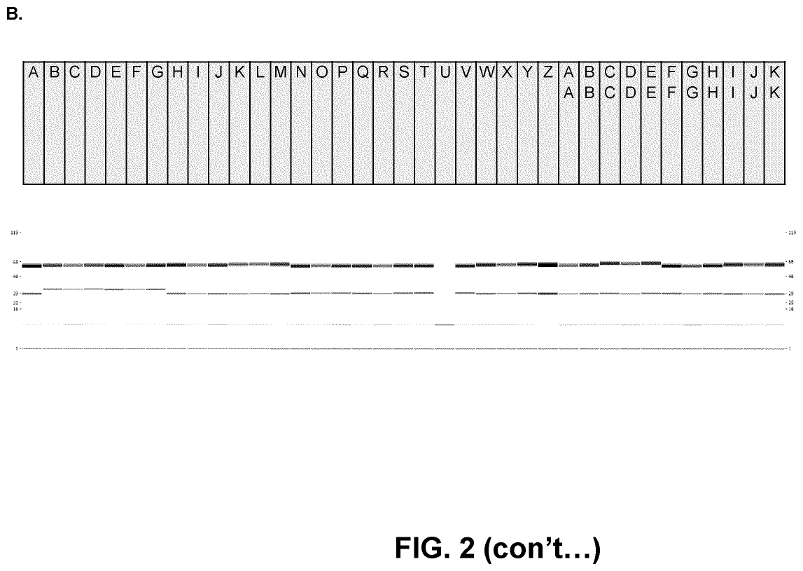

The results are shown in FIG. 2. Note that the intact non-reduced ADCs, including the unconjugated control (v17427), showed MWs of ˜160 kDa (as shown in FIG. 2(A)) rather than the calculated 150 kDa. This discrepancy is likely due either to the influence of binding dyes used in the LabChip™ protocol on the peptide bonds or to inherent accuracy limitations of the instrumentation (±20% size accuracy, based on manufacturer's protocol). Similarly, all the reduced samples, including the unconjugated control (v17427), showed MWs of ˜28 kDa for LC and ˜64 kDa for HC, both slightly higher than the calculated values of ˜23 kDa and 50 kDa, respectively (see FIG. 2(B)).

Comparing all 36 ADCs shown in FIG. 2 against the unconjugated control (v17427) indicates that the reduction-oxidation steps in the conjugation protocol were successful and that each antibody re-folded into the original full-length conformation prior to conjugation to the drug-linker.

### Example 8: On-Cell Antigen Binding Assay by Flow Cytometry

The apparent binding affinities of ADCs from Example 3 for their target c-Met, both as naked antibodies and as conjugates, were assessed by flow cytometry as described below and compared with the binding affinity of control unconjugated parental antibody (v17427).

Cells from the high c-Met-expressing cell line EBC-1 (4 million receptors/cell) (XenoTech, LLC, Lenexa, KS) were seeded at 25,000 cells/well with a minimum 100 uL seeding volume. Briefly, adherent EBC-1 cells were detached from their culture vessels using cell dissociation buffer and seeded in 96-well plates at 25,000 cells/well. Cells were kept on ice for 10 minutes, pelleted by centrifugation at 400g×3 minutes and supernatant was removed by flicking the inverted plate. Cell pellets were kept on ice. Test articles were titrated in cold FACS buffer and cells were treated with 50 uL per well of the designated treatment; the assay plate was parafilmed and incubated overnight at 4° C. Cells were then washed, incubated with secondary A647-Goat anti-Human Fc (Jackson ImmunoResearch Laboratories, Inc., West Grove, PA) at 2 μg/mL, and washed again. Cells were resuspended in 25 uL FACS buffer and analyzed using a BD LSRFortessa™ (X-20) High-Throughput Sampler (HTS) (BD Biosciences, San Jose, CA). GeoMean values obtained were then used to plot specific binding using Prism 8 software (GraphPad Prism Software) and used to calculate K_{D} and Bₘₐₓ values.

In this Example, all 10 cysteine insertion variants and both controls from Example 3 were assessed as naked antibodies and as the corresponding ADC comprising the MTvcCompound 1 drug-linker. Both controls and two selected cysteine insertion variants containing either a light or a heavy chain cysteine insertion (variants v27321 and v22765, respectively) were also tested as ADCs comprising the MCvcPABC-MMAE drug-linker. In addition, control v22758 (A114C) and the two selected cysteine insertion variants, v27321 and v22765, were tested as ADCs comprising the MCvcPAB-Tubulysin M drug-linker.

The results are shown in Table 8.1.

TABLE 8.1
Apparent K_{D} and Bₘₐₓ Values for Binding of
Naked Antibodies and ADCs to EBC-1 Cells¹

| | Insertion/ | | | Appar- |
|---|---|---|---|---|
| | Substitution | | | ent |
| Variant | Position | Drug-Linker | Bₘₐₓ | K_{d} (nM) |
| v17427 | None | None | 11163 | 0.10 |
| (Parent) | | | | |
| v17427 | None | MTvcCompound 1 | 12427 | 0.14 |
| (Parent) | | (stochastic DAR 4) | | |
| v22758 | H_A114C | None | 11348 | 0.16 |
| (Control) | | | | |
| v22758 | H_A114C | MTvcCompound 1 | 9929 | 0.26 |
| (Control) | | | | |
| v22758 | H_A114C | MCvcPABC-MMAE | 11742 | 0.18 |
| (Control) | | | | |
| v22758 | H_A114C | MCvcPAB-Tubulysin M | 11960 | 0.31 |
| (Control) | | | | |
| v29013 | H_S239.5C | None | 10080 | 0.16 |
| (Control) | | | | |
| v29013 | H_S239.5C | MTvcCompound 1 | 9162 | 0.22 |
| (Control) | | | | |
| v29013 | H_S239.5C | MCvcPABC-MMAE | 10654 | 0.21 |
| (Control) | | | | |
| v22760² | L_K39.5C | None | 11907 | 0.07 |
| v22760² | L_K39.5C | MTvcCompound 1 | 11557 | 0.22 |
| v22761² | L_K126.5C | None | 10097 | 0.13 |
| v22761² | L_K126.5C | MTvcCompound 1 | 11114 | 0.23 |
| v22765 | H_G237.5C | None | 12417 | 0.21 |
| v22765 | H_G237.5C | MTvcCompound 1 | 12097 | 0.20 |
| v22765 | H_G237.5C | MCvcPABC-MMAE | 11161 | 0.26 |
| v22765 | H_G237.5C | MCvcPAB-Tubulysin M | 11783 | 0.23 |
| v22768 | H_Q295.5C | None | 12335 | 0.16 |
| v22768 | H_Q295.5C | MTvcCompound 1 | 12290 | 0.23 |
| v27321 | L_W148.5C | None | 13536 | 0.23 |
| v27321 | L_W148.5C | MTvcCompound 1 | 12086 | 0.20 |
| v27321 | L_W148.5C | MCvcPABC-MMAE | 11562 | 0.25 |
| v27321 | L_W148.5C | MCvcPAB-Tubulysin M | 12695 | 0.37 |
| v27322 | L_K149.5C | None | 14520 | 0.34 |
| v27322 | L_K149.5C | MTvcCompound 1 | 12500 | 0.29 |
| v28983 | L_P40.5C | None | 12413 | 0.19 |
| v28983 | L_P40.5C | MTvcCompound 1 | 11870 | 0.20 |
| v28989 | H_A9.5C | None | 12111 | 0.19 |
| v28989 | H_A9.5C | MTvcCompound 1 | 12529 | 0.31 |
| v28993 | H_G169.5C | None | 12008 | 0.17 |
| v28993 | H_G169.5C | MTvcCompound 1 | 11290 | 0.22 |
| v29001 | H_T299.5C | None | 12112 | 0.57 |
| v29001 | H_T299.5C | MTvcCompound 1 | 10222 | 0.33 |

¹Control human IgG displayed no binding, as expected.
²Variant included the peptide ESSCDVKLV [SEQ ID NO: 2] fused to the C-terminal residue of the light chain

Overall, the cysteine insertion variants displayed comparable binding to the parental (v17427) naked antibody and the v17427-MTvcCompound 1 ADC. Variant v29001 and its MTvcCompound 1 conjugate, however, displayed lower binding (greater Kd, 5-fold) compared to control v17427.

All ADCs generally displayed very similar binding to their naked antibody counterpart. The MTvcCompound 1 ADCs of cysteine insertion variant v29001 and control v22758 displayed slightly lower Bₘₐₓ than their respective naked antibody counterparts.

Control v29013 (S239.5), both as naked antibody and as an ADC with either drug-linker, displayed lower Bₘₐₓ values compared to parental antibody (v17427).

Overall, this example demonstrates that, with the exception of variant v29001, ADCs comprising the cysteine insertion variants showed no differential binding compared to their naked antibody counterparts, and also showed comparable binding to parental antibody. Binding shown by the ADC comprising variant v29001, however, was still within a similar range to that of the corresponding ADCs comprising the controls v22758 and v29013.

### Example 9: In Vitro Cytotoxicity

Cytotoxicity of the ADCs from Example 3 comprising either MCvcPABC-MMAE or MTvcCompound 1 was tested in vitro against a variety of tumor cell lines expressing the target surface antigen (c-Met) as described below. The following cell lines were used (Table 9.1).

TABLE 9.1
Cell-lines used for Cytotoxicity Assay

| | | c-Met | Seeding | |
|---|---|---|---|---|
| | | Expression | Density | Incubation |
| Cell Line | Tumor Type | Level | (cells/well) | (days) |
| EBC-1 | Lung Carcinoma | High | 1500 | 4 |
| BT-20 | Breast Carcinoma | Low | 1000 | 4 |
| HCC827 | Lung Carcinoma | Mid | 700 | 4 |
| H292 | Lung Carcinoma | Mid | 700 | 4 |

Each of the cell lines shown in Table 9.1 was grown in the respective complete growth medium until assay day. After removal from the culture vessels using Trypsin-EDTA, cells were counted using a Scepter™ Cell Counter (Sigma-Aldrich Canada, Oakville, ON). Cells were diluted in complete growth medium to 20,000 cells/mL, such that 50 μL/well in 384-well plates equaled 1,000 cells/well, unless otherwise specified. All ADCs were diluted to 15 nM starting concentration in complete growth medium (RPMI 1640) followed by a 1:3 dilution in sterile 96-well dilution plates (final volume 200 μL/well). Samples (20 μL/well) were transferred into a 384-well plate in duplicate. Plates were then seeded with 20,000 cell/mL suspension of test cell line (50 μL/well), unless otherwise specified. Cells were allowed to settle at the bottom of wells by leaving at room-temperature for 5-10 min followed by incubation at 37° C./5% CO2 for 4 nights. After incubation, cell viability was quantified by adding 1×CellTiter-Glo® reagent (Promega Corporation, Madison, WI) at 10 μL/well. After incubating for 30 min in the dark, luminescence was measured using a Synergy™ H1 Hybrid Multi-Mode Reader (BioTek Instruments Inc., Winooski, VT). Data was analyzed with Prism 7 software (GraphPad Prism Software).

The results are shown in Table 9.2.

TABLE 9.2
EC₅₀ Values¹ for ADCs in C-Met Expressing Tumor Cell-lines

| | EC₅₀ (nM) | | | |
|---|---|---|---|---|
| ADC | EBC-1 | BT-20 | H292 | HCC827 |
| MMAE (free drug) | 0.16 | 0.13 | 0.11 | 0.36 |
| v22760²-MCvcPABC-MMAE | 0.08 | IC³ | >15 | >15 |
| v22761²-MCvcPABC-MMAE | 0.08 | 0.59 | >15 | >15 |
| v22765-MCvcPABC-MMAE | 0.05 | 2.03 | >15 | >15 |
| v22768-MCvcPABC-MMAE | 0.08 | 0.82 | >15 | >15 |
| v27321-MCvcPABC-MMAE | 0.07 | 1.65 | >15 | >15 |
| v27322-MCvcPABC-MMAE | 0.07 | 1.03 | >15 | >15 |
| v28983-MCvcPABC-MMAE | 0.05 | 0.64 | >15 | >15 |
| v28989-MCvcPABC-MMAE | 0.08 | 1.34 | >15 | >15 |
| v28993-MCvcPABC-MMAE | 0.07 | 8.13 | >15 | >15 |
| v29001-MCvcPABC-MMAE | 0.07 | 0.64 | >15 | >15 |
| v22758-MCvcPABC-MMAE | 0.08 | 0.58 | >15 | >15 |
| (Control) | | | | |
| v29013-MCvcPABC-MMAE | 0.04 | 1.27 | >15 | >15 |
| (Control) | | | | |
| Compound 1 (free drug) | 22.61 | 16.12 | 9.17 | 26.82 |
| v22760²-MTvcCompound 1 | 0.03 | 0.16 | 1.50 | 0.39 |
| v22761²-MTvcCompound 1 | 0.04 | 0.19 | 0.49 | 0.71 |
| v22765-MTvcCompound 1 | 0.03 | 0.11 | 0.46 | 0.24 |
| v22768-MTvcCompound 1 | 0.03 | 0.12 | 1.19 | 0.50 |
| v27321-MTvcCompound 1 | 0.03 | 0.15 | 1.04 | 0.85 |
| v27322-MTvcCompound 1 | 0.04 | 0.13 | >15 | 0.47 |
| v28983-MTvcCompound 1 | 0.02 | 0.08 | IC | 0.44 |
| v28989-MTvcCompound 1 | 0.03 | 0.09 | 0.39 | 0.32 |
| v28993-MTvcCompound 1 | 0.02 | 0.11 | 0.39 | 0.42 |
| v29001-MTvcCompound 1 | 0.03 | 0.09 | 0.40 | 0.31 |
| v22758-MTvcCompound 1 | 0.02 | 0.10 | 0.65 | 0.47 |
| (Control) | | | | |
| v29013-MTvcCompound 1 | 0.02 | 0.07 | 0.82 | 0.11 |
| (Control) | | | | |
| v17427-MTvcCompound 1 | 0.03 | 0.11 | 0.77 | 0.43 |
| (DAR 2 fraction) | | | | |
| (Wild-type control) | | | | |

¹n = 2
²Variant included the peptide ESSCDVKLV [SEQ ID NO: 2] fused to the C-terminal residue of the light chain
³IC = Test article showed cytotoxicity but curve obtained was not optimal for accurate EC₅₀ determination

As expected, ADCs comprising MTvcCompound 1 showed the greatest potency in the high c-Met expressing cell-line, EBC-1, compared to activity in lower c-Met expressing cell lines. All ADCs comprising this drug-linker, including the HIC-purified DAR 2 wild-type control (v17427-MTvcCompound 1), showed an EC₅₀ in the range 0.02-0.04 nM in the EBC-1 cell line. In the low c-Met expressing BT-20 cell line, higher potency was observed for cysteine insertion variants v28993 (EC50 0.11 nM), v29001 (EC50 0.12 nM), v28983 (EC₅₀ 0.12 nM), v28989 (EC50 0.13 nM), v22765 (EC50 0.14 nM) and v27321 (EC50 0.14 nM) than for the wild-type control (v17427-MTvcCompound 1 DAR 2 fraction) (EC₅₀ 0.15 nM) signifying the importance of site-specific conjugation over stochastic conjugation. All ADCs comprising the MTvcCompound 1 drug-linker showed EC50>15 nM in the mid c-MET expressing H292 cell line. For the mid c-Met expressing HCC827 cell line, the cysteine insertion variants conjugated to MTvcCompound 1 showed EC₅₀ values in the range 0.24-0.71 nM. The control ADC, v29013 (S239.5) conjugated to MTvcCompound 1, showed the highest in vitro potency (EC₅₀ 0.16 nM) in this cell line.

For ADCs comprising MCvcPABC-MMAE, control v29013 (S239.5) showed highest potency in the high c-Met expressing cell line, EBC-1 (EC₅₀ 0.04 nM). All 10 cysteine insertion variants conjugated to MCvcPABC-MMAE showed potencies in the range EC₅₀ 0.05-0.09 nM. In the low c-MET expressing BT-20 cell line, higher potency was observed for cysteine insertion variants v22761 (EC₅₀ 0.59 nM), v29001 (EC₅₀ 0.64 nM), v28983 (EC₅₀ 0.64 nM) and v27322 (EC₅₀ 1.03 nM) conjugated to MCvcPABC-MMAE than for the control, v29003 (S239.5) conjugated to MCvcPABC-MMAE (EC₅₀ 1.27 nM). All ADCs comprising the MCvcPABC-MMAE drug-linker showed EC₅₀ values >15 nM in the mid c-Met expressing cell lines H292 and HCC827.

Overall, this Example demonstrates that the potency of ADCs comprising cysteine insertion variants does not appear to be affected by the position of the individual cysteine insertion site.

### Example 10: Stability of Antibody Drug Conjugates in Mouse Plasma

For multiple reasons, including enzymatic metabolism and retro-Michael reactions, an ADC can lose its payload while in circulation in vivo or the payload may be modified in a manner that renders the ADC ineffective. The ADCs from Example 3 were assessed in a mouse plasma stability assay as described below to determine loss of payload (drug-linker).

ADCs were diluted in mouse plasma at 0.5 mg/mL and incubated in a water bath at 37° C. for 0, 1, 3 and 7 days, before drug loss for each was assessed. Samples were removed from the water bath at the noted time points and immediately frozen at −80° C. Time points within 24h were separately prepared for ADCs comprising the MCvcPAB-Tubulysin M drug-linker. ADC and antibody were recovered by immunoprecipitation. Samples were first deglycosylated with 250 ng of EndoS enzyme (2 ug ADC in 50 uL PBS) for 1 hr at room temperature (RT). Deglycosylated ADCs were then captured onto streptavidin magnetic beads (GE Healthcare Life Sciences, Chicago, IL) pre-coupled to biotinylated goat anti-human IgG F (ab′)2 capturing antibody (Jackson Immunoresearch Laboratories, Inc., West Grove, PA), 15 ug capturing antibody per 100 uL magnetic bead slurry per sample, for 1.5 hrs at RT. Following ADC capture, samples were reduced with 25 mM DTT (ThermoFisher Scientific, Waltham, MA) per 100 μL of sample for 1 hr at RT, and then eluted in 20 μL of elution buffer (20% acetonitrile, 1% formic acid, in dH₂O) for 1 hr at RT. Control ADC (v22758) spiked into mouse plasma at 2.0 ug was included as control to validate the immunoprecipitation procedure. DAR for each sample was assessed by LC-MS as described in General Procedure 6 in order to determine the amount of drug-linker loss.

In addition to potential loss of drug-linker, the maleimide ring in the linker can potentially undergo water-mediated ring opening, which in turn stabilizes the ADC. Maleimide ring opening would lead to an increase of 18 Da in the ADC mass. For all samples, the amount of maleimide ring opening was calculated in addition to drug-linker loss.

Tubulysin M is susceptible to metabolism via loss of an acetyl group while in circulation. Understanding whether this type of decomposition occurs in the cysteine insertion variant ADCs provides additional information regarding the stability/exposure/accessibility of the respective cysteine insertion site. To assess whether any of the cysteine insertion sites helps protect the Tubulysin M payload from the acetyl loss, plasma stability was monitored and compared against the ADCs comprising controls v22758 (Thiomab HC-A114C) and v29013 (S239.5). In general, % decomposition for Tubulysin M ADCs was calculated as the proportion of all drug-loaded species having lost the acetyl group mass (ring-opened and non-ring-opened) divided by the sum of all drug-loaded species.

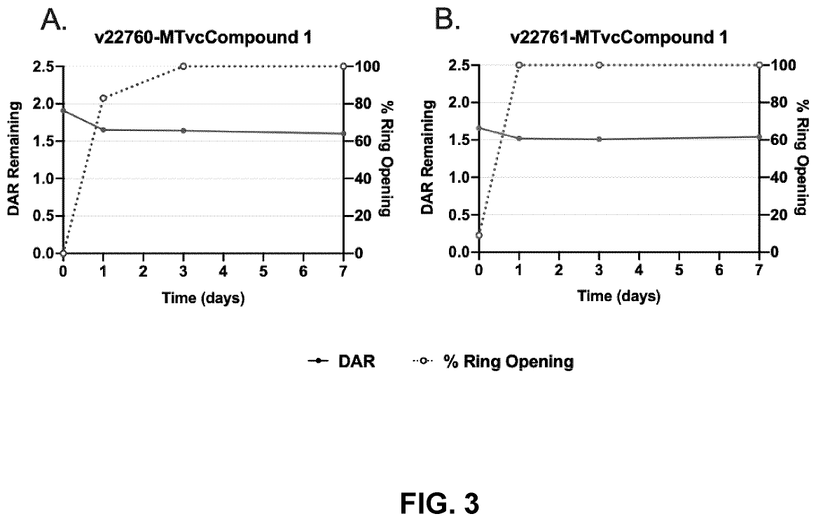

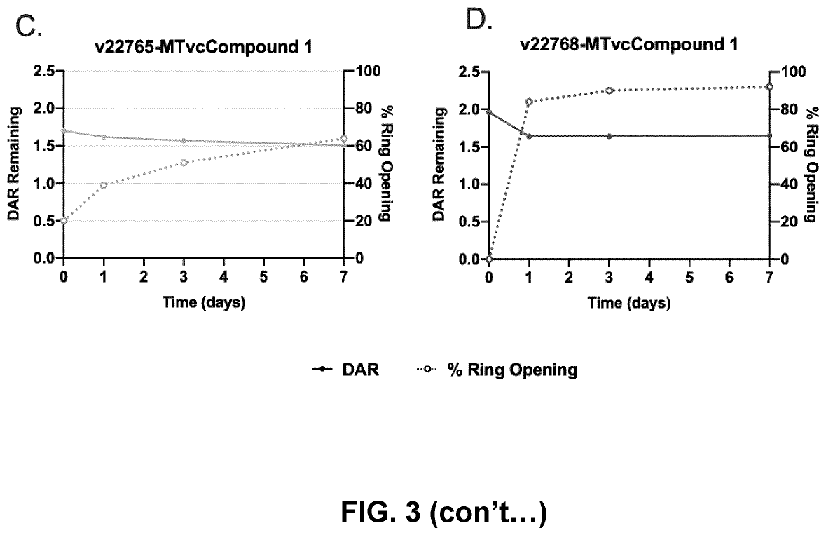

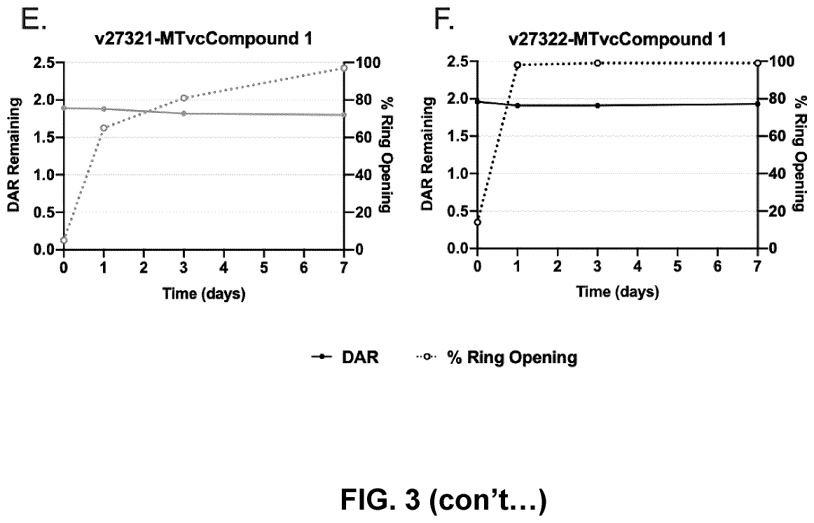

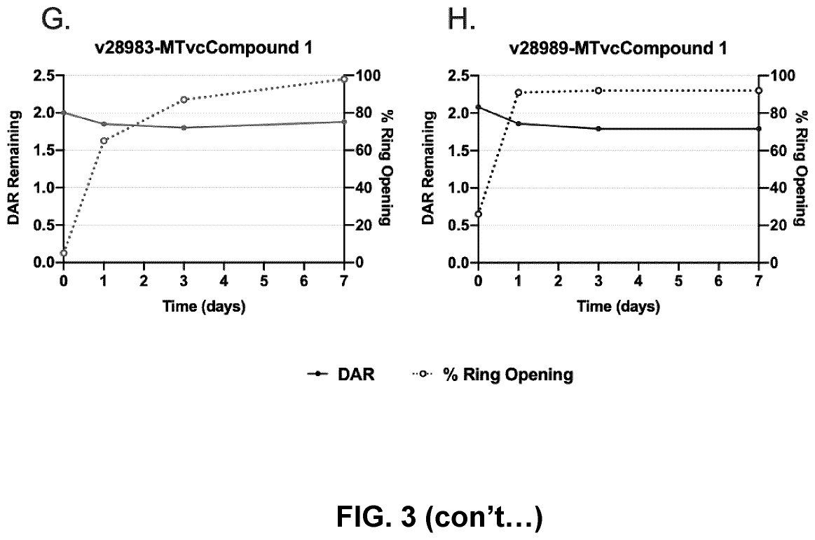

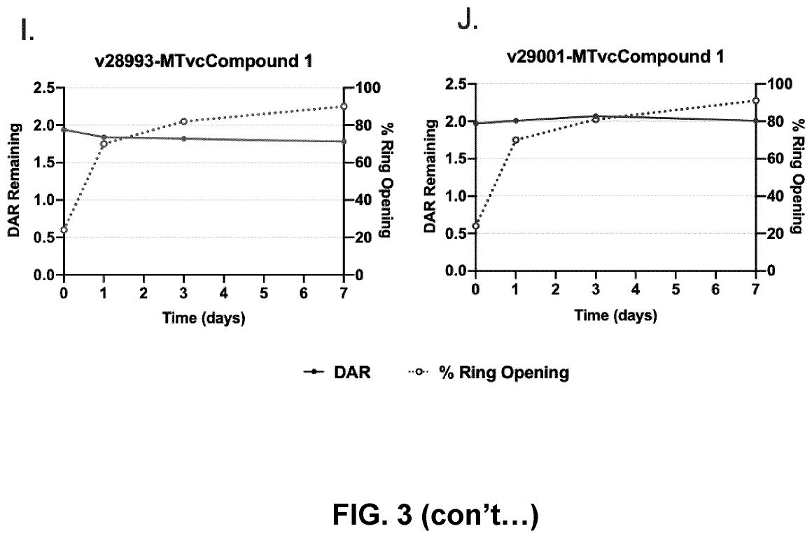

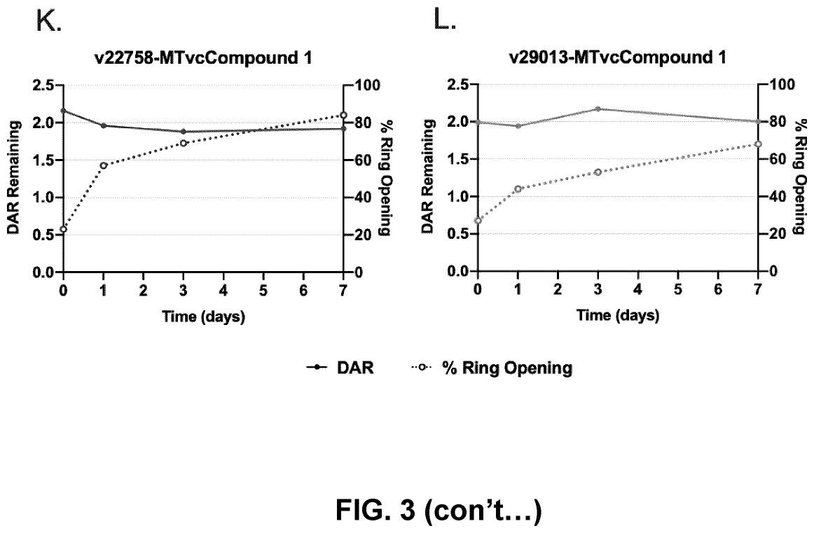

The results of the stability studies are shown in FIGS. 3, 4 and 5.

As can be seen from FIG. 3, for the ADCs comprising the drug-linker MTvcCompound 1, DAR loss was similar across most variants, with the largest decrease occurring in the first 24 hours and reaching a final DAR ˜1.6 by day 7. For ADCs comprising variants v27322, v29001 and control v29013, the DAR loss was nearly negligible over the entire incubation period. Maleimide ring opening for most variants started at 0-20% and progressed to fully ring-opened by 7 days. ADCs comprising variants v22765 and control v29013 reached only ˜70% ring opening by day 7.

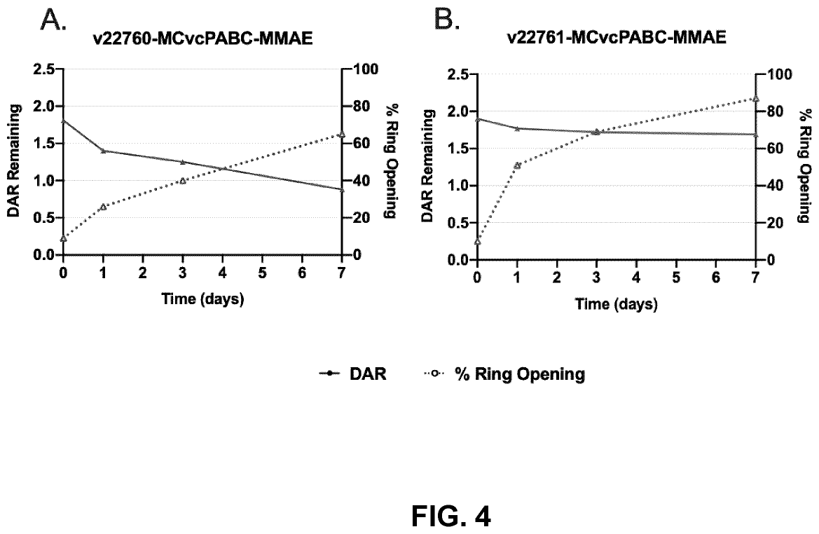

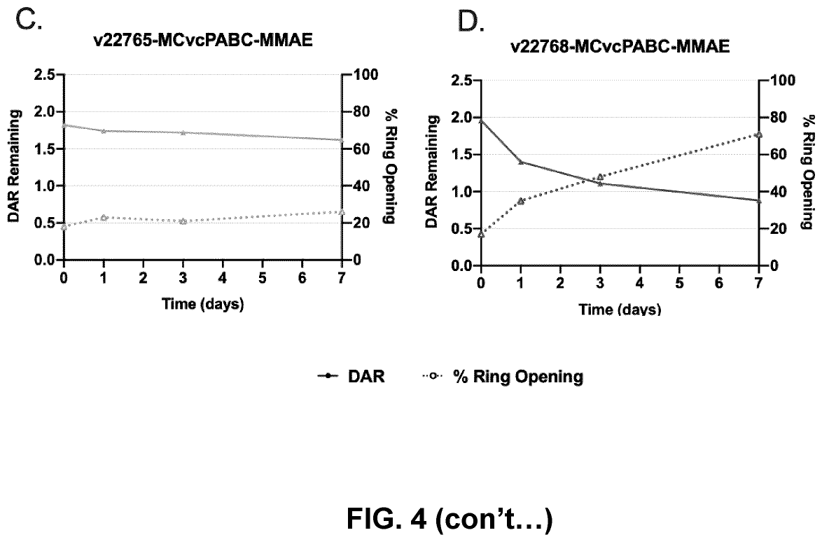

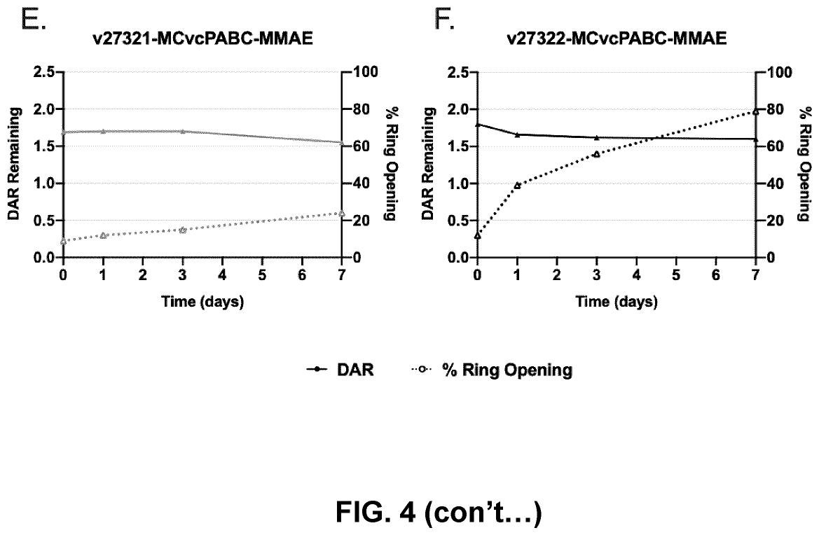

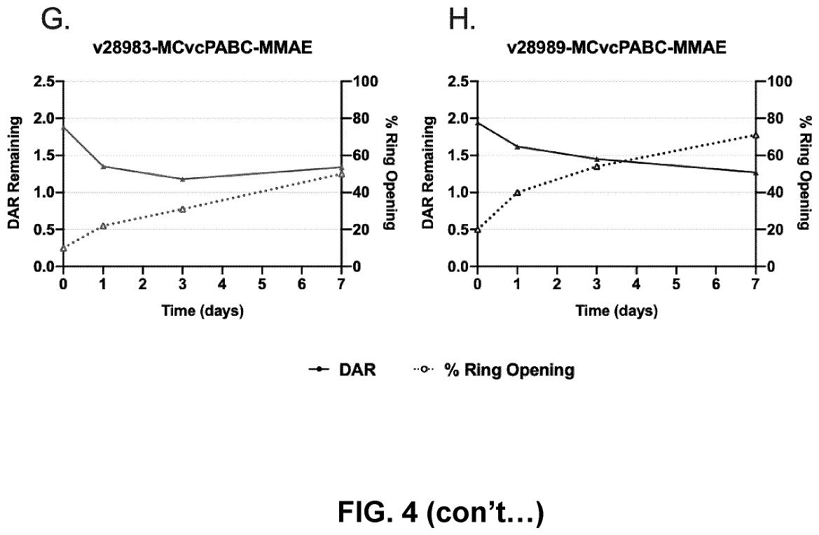

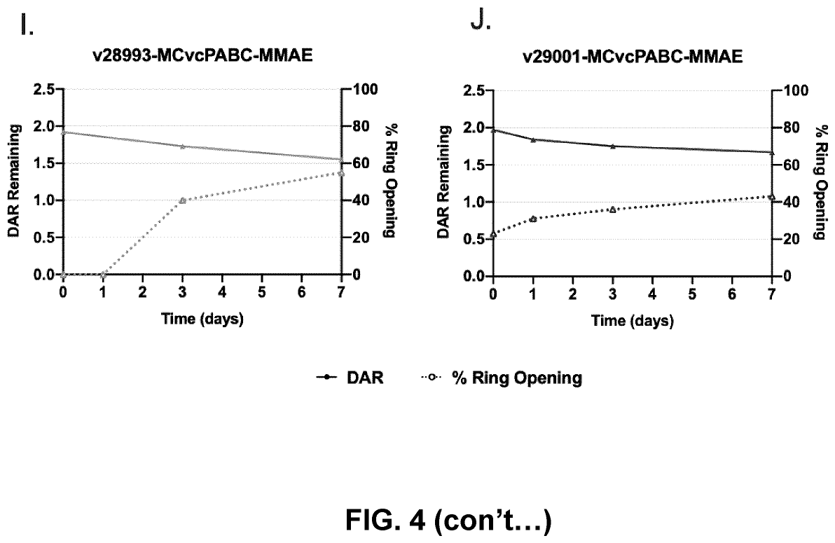

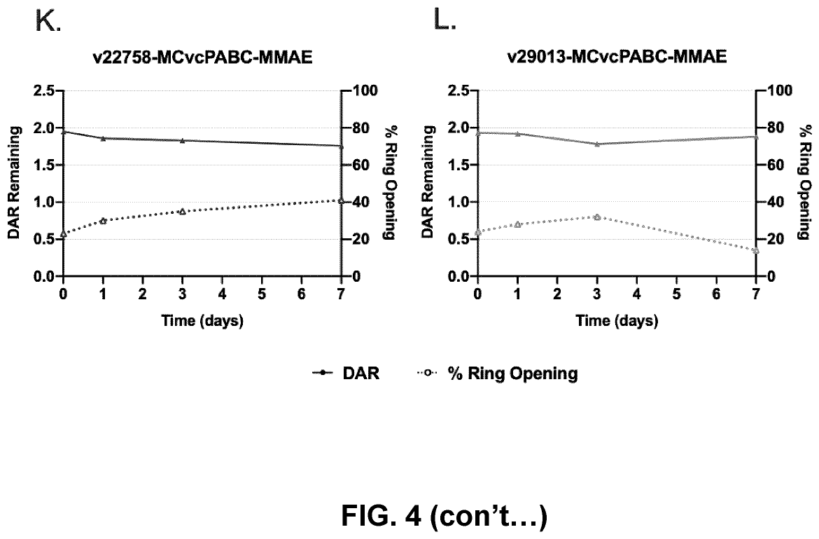

FIG. 4 shows that for the ADCs comprising the drug-linker MCvcPABC-MMAE, DAR loss was ˜10% for most variants over the incubation period. The least stable ADCs were those comprising variants v22760 and v22768, which lost 50% DAR over 7 days. For the MCvcPABC-MMAE ADCs, ring opening and DAR loss did not entirely correlate: ADCs comprising variants v22760 and v22768 showed ˜70% ring opening but also showed the highest DAR loss. The most stable ADCs were those comprising the controls v29013 and v22758, and those comprising the cysteine insertion variants v22761, v22765, v27321 and v27322, all of which showed <10% DAR loss.

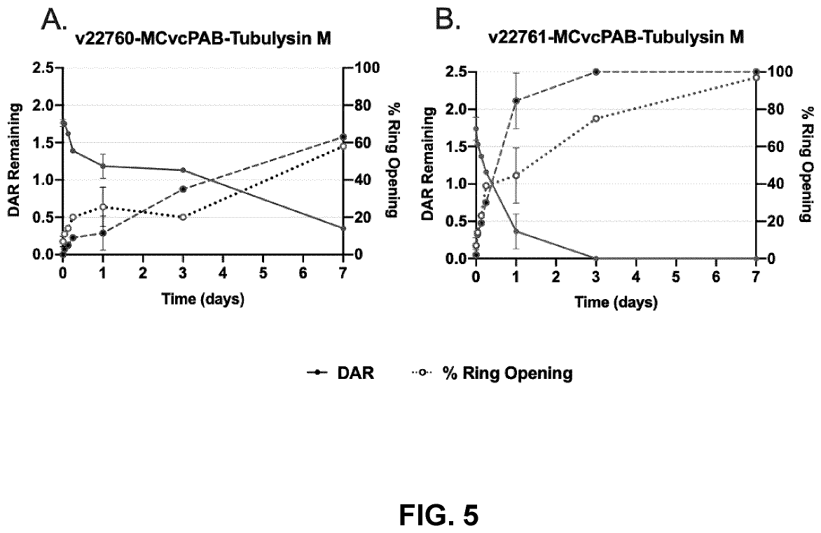

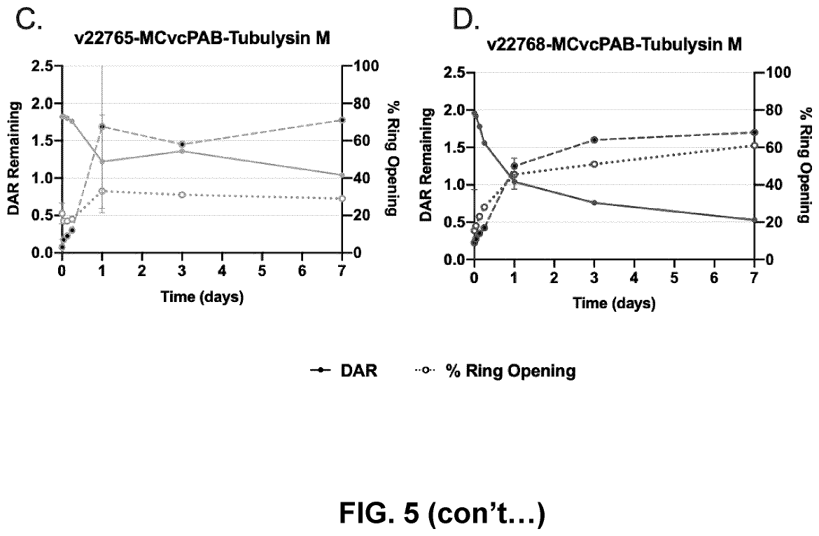

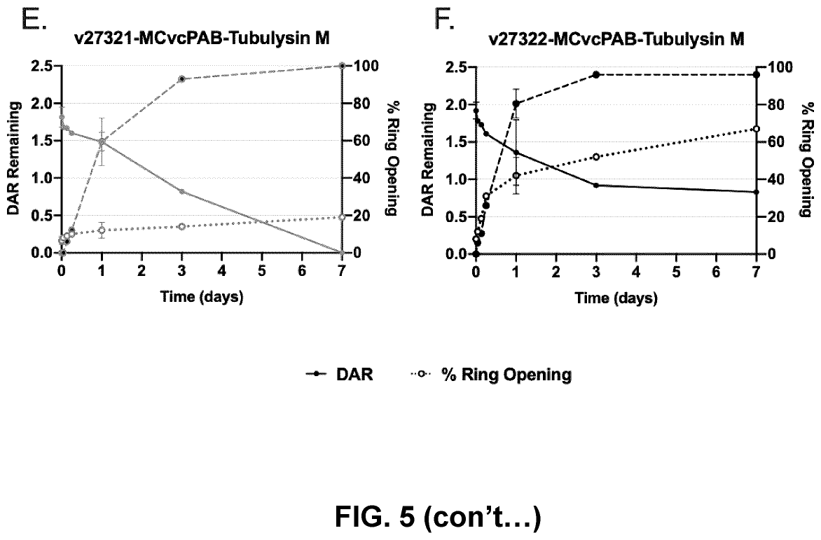

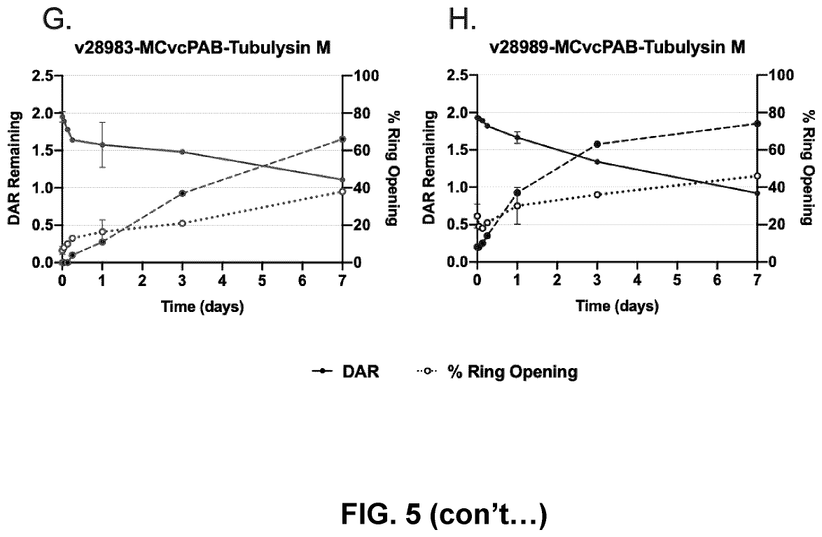

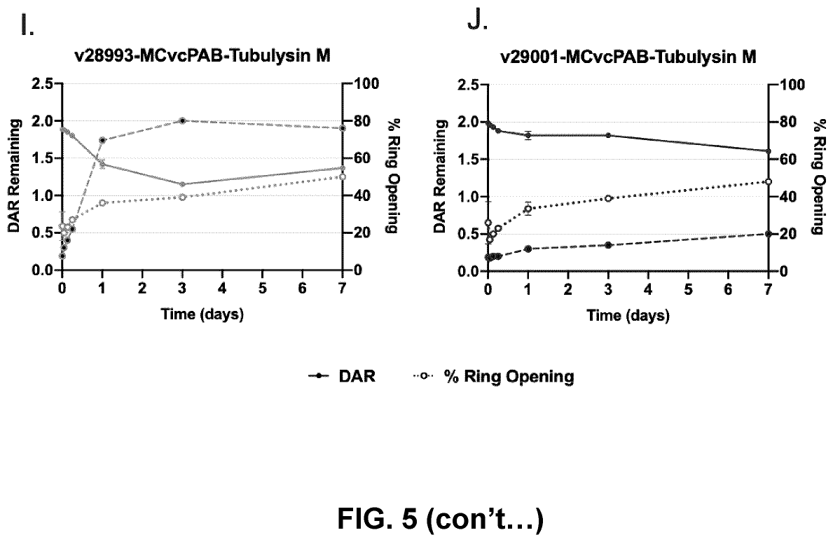

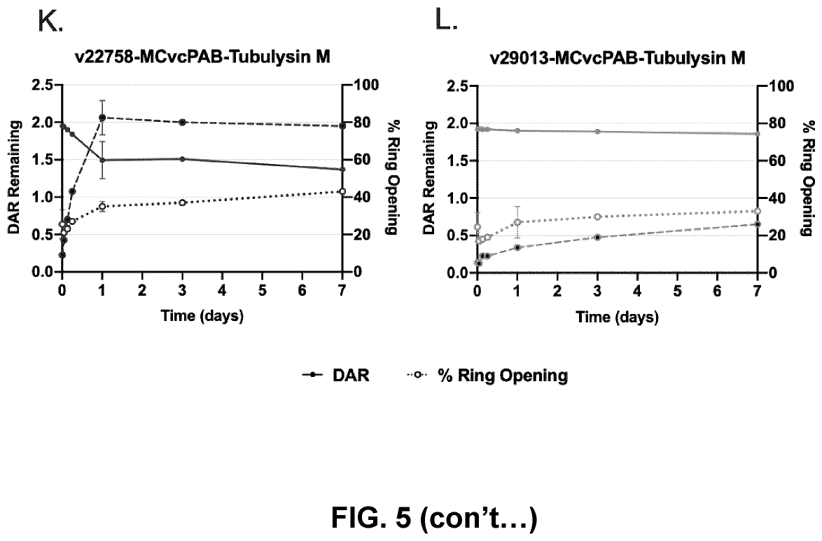

FIG. 5 shows that among the ADCs comprising the drug-linker MCvcPAB-Tubulysin M, those comprising variants v22761, v27321, v27322, and control v22758 displayed rapid Tubulysin M decomposition, with >70% decomposition at 24 h and 100% by day 7. The majority of the DAR loss in ADCs comprising variants v22761 and v27321 was due to this decomposition. The most stable MCvcPAB-Tubulysin M ADC comprised variant v29001 which displayed only ˜20% decomposition over 7 days, moderate ring opening, and very little DAR loss. Decomposition in this ADC was 5% less than the amount of decomposition displayed by the control v29013-MCvcPAB-Tubulysin M ADC.

### Example 11: Preparation of Dar-Tuned Antibody Drug Conjugates

The ADCs described in the previous Examples had an average DAR of ˜2, with the same cysteine insertion on either both heavy chains (1xcys HC) or both light chains (1xcys LC). In this Example, cysteine insertions were evaluated for potential combinations to generate constructs with more or less than two cysteine residue insertions per antibody allowing for generation of ADCs having an average DAR of 1, 2 or 3 as described below.

Constructs containing one insertion per antibody molecule (1xcys Ab) were generated by means of heterodimeric assembly of the heavy chains. These constructs contained one heavy chain without any insertion and one heavy chain with a single inserted cysteine residue (1xcys HC).

Constructs containing three insertions per antibody molecule (3xcys Ab) were generated by means of heterodimeric assembly of the heavy chains. This was achieved either by combining two light chains having a single cysteine insertion each (1xcys LC) with one heavy chain having a single cysteine insertion (1xcys HC) and one heavy chain without any insertion, or by combining one heavy chain having a single cysteine insertion (1xcys HC) with one heavy chain having two cysteine insertions (2xcys HC).

Details are provided in Table 11.1.

In addition, constructs with four inserted cysteines per antibody molecule (4xCys Ab) may be created by combining two different sets of insertion designs-either by combining two light chains having a single cysteine insertion each (1xCys LC) with two heavy chains having a single cysteine insertion each (1xCys HC), or by combining two heavy chains having two cysteine insertions each (2xCys HC) or two light chains having two cysteine insertions each (2xCys LC).

TABLE 11.1
Selected Cysteine Insertion Sites and Expected DAR Values

| | | | Cysteine Insertion Site | | |
|---|---|---|---|---|---|
| | | | | | Light |
| Variant | Variant | Target | Heavy | Heavy | Chain |
| (ADC) | (mAb) | DAR | Chain A | Chain B | (2x) |
| v34293 | v17427 | 2 | None | None | None |
| | (parental | (stochastic)¹ | | | |
| | antibody) | | | | |
| v34276 | v33967 | 1 | H_T299.5C | None | None |
| v34277 | v33968 | | H_G237.5C | None | None |
| v34278 | v33969 | | H_A9.5C | None | None |
| v34279 | v29001 | 2 | H_T299.5C | H_T299.5C | None |
| v34280 | v28983 | | None | None | L_P40.5C |
| v34281 | v33970 | 3 | H_T299.5C | None | L_P40.5C |
| v34282 | v33971 | | H_G237.5C, | H_T299.5C | None |
| | | | H_T299.5C | | |
| v34284 | v33974 | | H_A9.5C, | H_T299.5C | None |
| | | | H_T299.5C | | |
| v34285 | v33977 | | H_A9.5C | None | L_P40.5C |
| v34286 | v33979 | | H_A9.5C, | H_A9.5C | None |
| | | | H_G237.5C | | |

¹Lysine conjugation

Antibodies were prepared as described in General Procedure 1, and each antibody was conjugated to MTvcCompound 1 as described in General Procedure 2 with exception of v17427 which was stochastically conjugated to an NHS-ester activated Compound 1 at lysine residues at DAR2 to produce ADC v34293.

In vitro characterization of each resulting “DAR-tuned” ADC by HIC, UPLC-SEC, LC-MS and CE-SDS was conducted as described in General Procedures 4-7. The results are shown in Table 11.2.

TABLE 11.2
In vitro Characterization of DAR tuned ADCs

| | | | | | UPLC-SEC | | |
|---|---|---|---|---|---|---|---|
| Variant | Variant | Target | HIC | DAR | Monomer | HMWS | LMWS |
| (ADC) | (mAb) | DAR | RRT | (MS) | % | % | % |
| v34293 | v17427 | 2 | NA | 2.07 | 95 | 5 | 0 |
| | (parental | (stochastic)¹ | | | | | |
| | antibody) | | | | | | |
| v34276 | v33967 | 1 | 1.02 | 1.0 | 99.2 | 0 | 0.8 |
| v34277 | v33968 | | 1.06 | 0.9 | 97.7 | 0.8 | 1.5 |
| v34278 | v33969 | | 1.04 | 1.0 | 96.6 | 0.9 | 2.6 |
| v34279 | v29001 | 2 | 1.02 | 2.0 | 96.4 | 0.9 | 2.7 |
| v34280 | v28983 | | 1.09 | 1.9 | 95.3 | 0.4 | 0 |
| v34281 | v33970 | 3 | 1.07 | 3.0 | 96.1 | 2.4 | 1.5 |
| v34282 | v33971 | | 1.05 | 2.8 | 92.5 | 1.4 | 6.1 |
| v34284 | v33974 | | 1.11 | 2.8 | 92.8 | 1.3 | 5.9 |
| v34285 | v33977 | | 1.2 | 2.9 | 94.7 | 3.4 | 1.9 |
| v34286 | v33979 | | 1.15 | 2.8 | 97.7 | 1.6 | 0.7 |

¹Lysine conination

As can be seen from Table 11.2, all variants were successfully conjugated to MTvcCompound 1 at their respective target DAR as determined by LC-MS. HIC-RRT values for each ADC correlated well with estimated values for DAR 2 MTvcCompound 1 ADCs (see Examples 4 and 6). UPLC-SEC profiles showed >90% monomer for each of the DAR-tuned ADCs.

CE-SDS showed that all DAR-tuned ADCs appeared mostly as full-sized antibodies with insignificant amounts of non-specific conjugation.

### Example 12: On Cell Binding Assay-Dar-Tuned ADCS

In vitro characterization of the ADCs from Example 11 by on cell binding was evaluated as described in Example 8 on cMet-expressing cell lines EBC-1, H292 and BT-20. ADC v34281 (DAR 3) was tested in EBC-1 and H292 cell lines only. Parental antibody (v17427) conjugated to the drug-linker ADvcCompound 1 at DAR 2 (stochastic, lysine conjugation) or MTvcCompound 1 at DAR 4 (stochastic, cysteine conjugation) were included as additional controls.

The results are shown in Table 12.1. As expected, all DAR-tuned ADCs showed similar target binding to the unconjugated parental antibody (v17427), regardless of DAR.

TABLE 12.1
On-Cell Binding of DAR-Tuned ADCs

| | | | EBC-1 | | H292 | | BT-20 | |
|---|---|---|---|---|---|---|---|---|
| Variant | Variant | Target | | Apparent | | Apparent | | Apparent |
| (ADC) | (mAb) | DAR | Bmax | Kd (nM) | Bmax | Kd (nM) | Bmax | Kd (nM) |
| — | v17427 | — | 5,604 | 0.14 | 1,398 | 0.02 | 2,451 | 0.02 |
| v34276 | v33967 | 1 | 5,588 | 0.14 | 1,423 | 0.02 | 2,256 | 0.02 |
| v34277 | v33968 | | 5,731 | 0.13 | 1,435 | 0.01 | 2,276 | 0.02 |
| v34278¹ | v33969 | | 5,813 | 0.28 | 1,431 | 0.03 | 2,334 | 0.03 |
| v34279 | v29001 | 2 | 5,707 | 0.13 | 1,367 | 0.01 | 2,255 | 0.01 |
| v34280 | v28983 | | 5,455 | 0.16 | 1,432 | 0.01 | 2,241 | 0.01 |
| v34281 | v33970 | 3 | 5,345 | 0.13 | 1,335 | 0.01 | 2,141 | 0.01 |
| v34282 | v33971 | | 5,806 | 0.14 | 1,286 | 0.01 | 2,188 | 0.01 |
| v34284 | v33974 | | 5,611 | 0.23 | 1,306 | 0.02 | 2,047 | 0.01 |
| v34285 | v33977 | | 5,422 | 0.17 | 1,382 | 0.02 | 2,352 | 0.02 |
| v34286 | v33979 | | 5,441 | 0.20 | 1,297 | 0.02 | 2,443 | 0.02 |
| — | v22277 | — | 138 | >80 | 13 | >10 | 32 | >10 |

¹DAR of the ADC v34278 used in this experiment was 1.4

### Example 13: In Vitro Cytotoxicity-DAR-TUNED ADCS

In vitro cytotoxicity of the ADCs from Example 11 was assessed as described in Example 9. Parental antibody (v17427) conjugated to the drug-linker MTvcCompound 1 at DAR 4 (stochastic, cysteine conjugation) was included as an additional control.

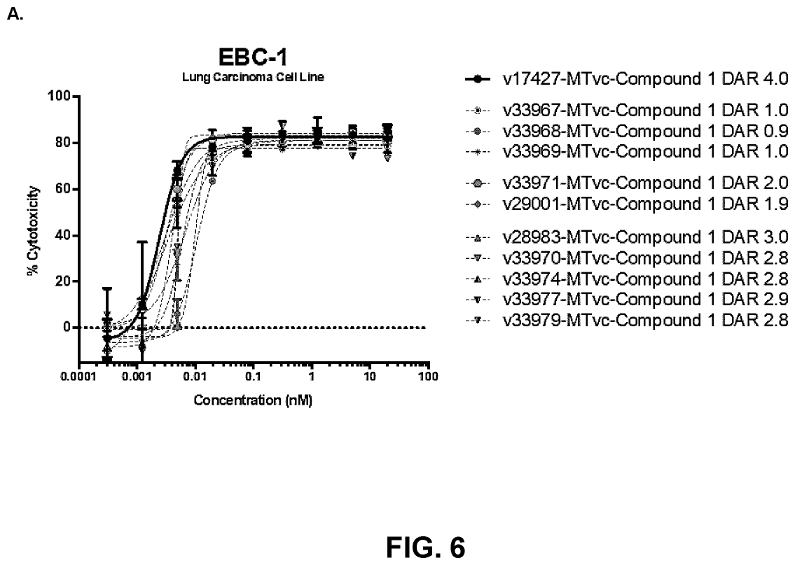

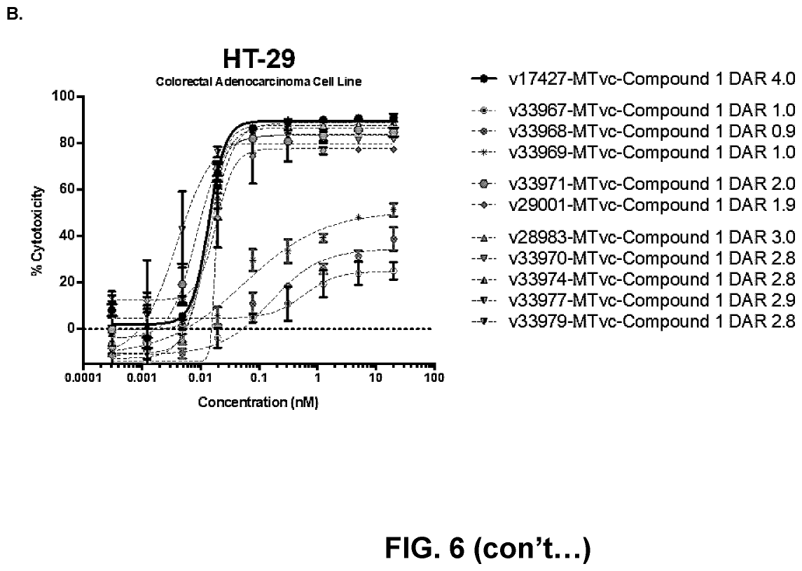

The results are shown in FIG. 6 and Table 13.1. In general, the cytotoxicity of the DAR 3 ADCs was greater than that of the DAR 2 and DAR 1 ADCs. Between the DAR 1 ADCs, no significant differences were observed in cytotoxicity.

TABLE 13.1
EC₅₀ Values for ADCs in cMet Expressing Tumor Cell-lines

| ADC | | | EC₅₀ (pM) | |
|---|---|---|---|---|
| Variant | Sample | DAR | EBC-1 | HT-29 |
| — | Compound 1 (free drug) | — | 55,320 | 66,850 |
| v34276 | v33967-MTvc-Compound 1 | 1.0 | 2.7 | 585.9 |
| v34277 | v33968-MTvc-Compound 1 | 0.9 | 10.5 | 156.4 |
| v34278 | v33969-MTvc-Compound 1 | 1.0 | 5.9 | 54.8 |
| v34282 | v33971-MTvc-Compound 1 | 2.0 | 3.2 | 8.4 |
| v34279 | v29001-MTvc-Compound 1 | 1.9 | 3.1 | 18.4 |
| v34280 | v28983-MTvc-Compound 1 | 3.0 | 3.9 | 13.7 |
| v34281 | v33970-MTvc-Compound 1 | 2.8 | 6.7 | 18.1 |
| v34284 | v33974-MTvc-Compound 1 | 2.8 | 5.0 | 13.9 |
| v34285 | v33977-MTvc-Compound 1 | 2.9 | 9.9 | 12.9 |
| v34286 | v33979-MTvc-Compound 1 | 2.8 | 5.0 | 3.9 |
| — | v17427-MTvc-Compound 1 | 4.0 | 2.4 | 13.8 |

Overall, the results show that the DAR-tuned ADCs were all active against the c-Met expressing cell-lines EBC-1 and HT-29. The in vitro potency of these ADCs correlated well with the DAR values (i.e. the number of conjugated toxins).

### Example 14: In vivo Anti-Tumor Activity

A selection of ADCs from Example 11 was assessed for in vivo anti-tumor activity in the high c-Met expressing non-small cell lung cancer xenograft model H1975 and in the mid c-Met expressing colorectal cancer xenograft model HT-29. Activities of the control ADCs v17427-MCvcPABC-MMAE (DAR4), v17427-MTvc-Compound 1 (DAR4) and v17427-ADvc-Compound 1 (DAR2) were assessed for comparison.

For the HT-29 model, tumor cell suspensions (3×10⁶ cells in 0.1 ml PBS) were implanted subcutaneously into balb/c nude mice. When mean tumor volume reached ˜160 mm3, the animals were randomly assigned to groups (n=8 per group) and treated with a single IV dose of test articles as shown in Table 14.1. Dose levels of ADCs at different DARs were molar matched to toxin. Tumor volume and body weight were measured twice weekly with a study duration of 32 days.

For the H1975 model, tumor cell suspensions (5×10⁶ cells in 0.1 ml PBS) were implanted subcutaneously into balb/c nude mice. When mean tumor volume reached ˜150 mm3, the animals were randomly assigned to groups (n=8 per group) and treated with a single IV dose of test articles as shown in Table 14.1. Tumor volume and body weight were measured twice weekly with a study duration of 38 days.

TABLE 14.1
Doses for in vivo Study

Doses (mg/kg)

| Test Article | DAR | HT-29 | H1975 |
|---|---|---|---|
| Vehicle | | 0 | 0 |
| v33967-MTvc-Compound 1 | 1 | 12, 6 | 24, 4 |
| v33968-MTvc-Compound 1 | 1 | 12, 6 | 24, 4 |
| v29001-MTvc-Compound 1 | 2 | 6, 3 | 12, 2 |
| v28983-MTvc-Compound 1 | 2 | 6, 3 | 12, 2 |
| v17427-ADvc-Compound 1 | 2 | 6, 3 | 12, 2 |
| (control) | | | |
| v33970-MTvc-Compound 1 | 3 | 4, 2 | 8, 1.3 |
| v33971-MTvc-Compound 1 | 3 | 4, 2 | 8, 1.3 |
| v33979-MTvc-Compound 1 | 3 | 4, 2 | 8, 1.3 |
| v17427-MTvc-Compound 1 | 4 | 3, 1.5 | 6, 1 |
| (control) | | | |
| v17427-MCvcPABC-MMAE | 4 | 1.5 | 6 |
| (control) | | | |

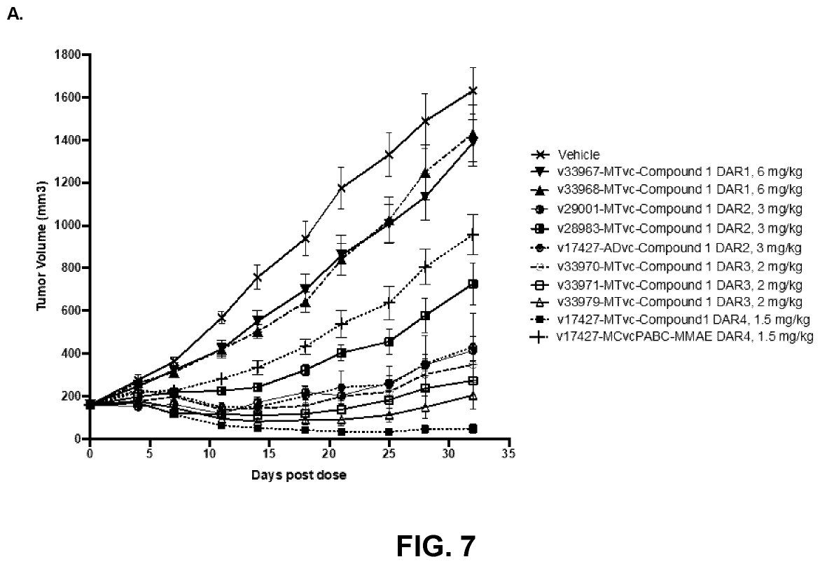

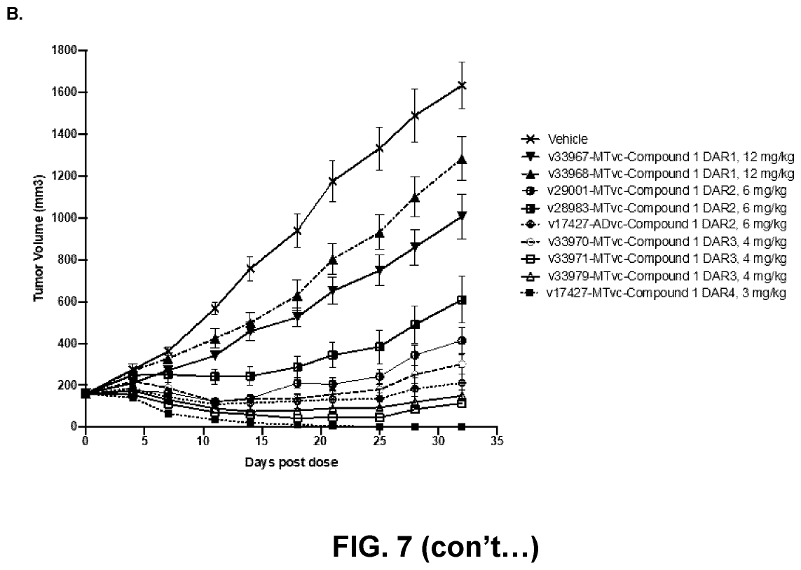

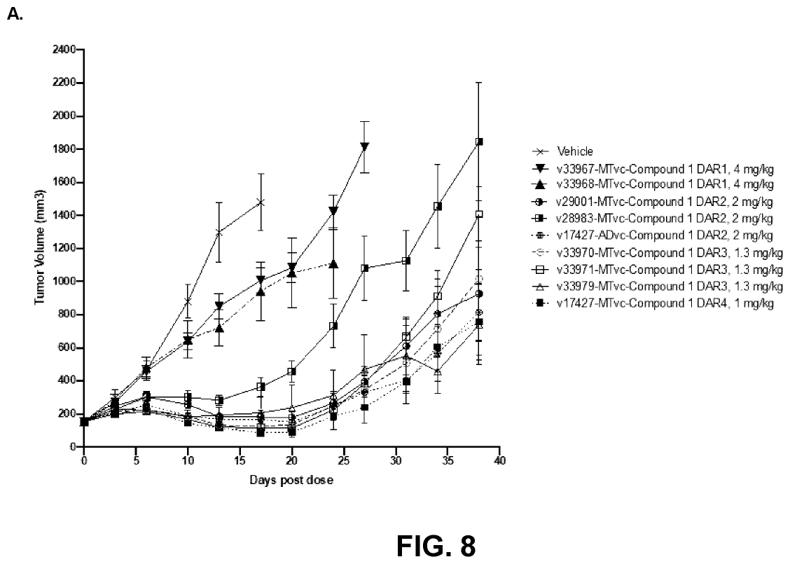

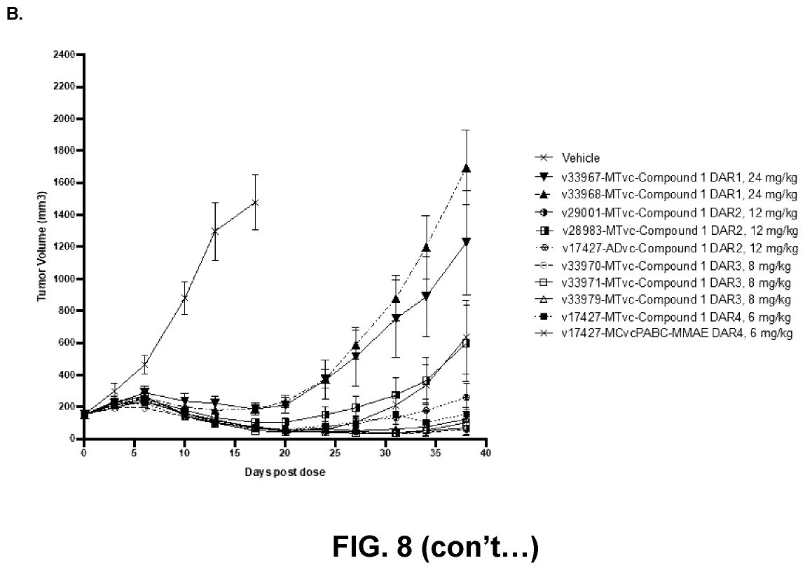

The results are shown in FIG. 7 and FIG. 8.

In the HT-29 model, all site-specific and stochastic conjugated DAR2, DAR3 and DAR4 Compound 1 ADCs significantly inhibited tumor growth compared to vehicle at the toxin-matched antibody doses of 6, 4 and 3 mg/kg respectively (p<0.05, mixed effects model for tumor growth rate) (FIG. 7B). DARI Compound 1 ADCs did not inhibit tumor growth at the toxin-matched dose of 12 mg/kg (FIG. 7B). All site-specific and stochastic conjugated DAR2, DAR3 and DAR4 Compound 1 ADCs, with the exception of v28983-MTvc-Compound 1, also significantly inhibited tumor growth compared to vehicle at the toxin-matched antibody doses of 3, 2 and 1.5 mg/kg respectively (FIG. 7A). By comparison, v17427-MCvcPABC-MMAE did not significantly inhibit tumor growth at the 1.5 mg/kg dose tested (FIG. 7A). There was a trend for a positive correlation of DAR with anti-tumor activity, when antibody dose was toxin-matched. Activity of the site-specific v29001-MTvc-Compound 1 DAR2 ADC was comparable to that of the v17427-ADvcCompound 1 stochastic DAR2 control (FIG. 7A).

For the H1975 model, all DARI, DAR2, DAR3 and DAR4 Compound 1 ADCs significantly inhibited tumor growth compared to vehicle at the toxin-matched antibody doses of 24, 12, 8 and 6 mg/kg respectively (p<0.05, mixed effects model for tumor growth rate) (FIG. 8B). While all DAR2, DAR3 and DAR4 Compound 1 ADCs also significantly inhibited tumor growth compared to vehicle at the toxin-matched antibody doses of 2, 1.3 and 1 mg/kg respectively, DAR1 ADCs did not significantly inhibit tumor growth at the toxin-matched antibody dose of 4 mg/kg (FIG. 8A).

No significant body weight loss was observed in any treatment group in either study.

### Example 15: FcγR AND FcRn Binding of Cysteine Insertion Variants

The 10 cysteine insertion variants shown in Table 2.2 were assessed for their ability to bind to the neonatal Fc receptor (FcRn) and the Fcγ receptors (FcγR) CD64a (FcγRI), CD32a (FcγRIIA; allelic forms His131 and Arg131), CD32b (FcγRIIB) and CD16a (FcγRIIIA; allelic forms V158 and F158) as described below.

Binding to FcγRs: Affinity of FcγRs for the tested variants was measured by surface plasmon resonance (SPR) using the Biacore™ T200 System (Cytiva, Marlborough, MA) with PBS buffer pH 7.4 containing 0.05% Tween 20 and 3.4 mM EDTA. Protein A (Genscript Biotech Corporation, Piscataway, NJ; Cat. Z02201) at 15 ug/mL in 10 mM sodium acetate pH 4.5 was covalently immobilized on a CM5 sensor chip through standard amine coupling to 2000 RU (response units). Each test variant at 2.5 ug/mL was injected at a flow rate of 10 uL/min for 30s for Protein A capture. FcγRs were injected at 25 uL/min over the antibody-immobilized surface using single cycle kinetics. For CD32aH, CD32aR, and CD32bY, which have weak affinity and fast on and off interactions, 15s of increasing concentrations between 0.15 and 12 uM were used. For CD16aF and CD16aV, 40s injections of increasing concentrations between 0.06 and 5 uM were used. For CD64a, 100s injections of increasing concentrations between 0.41 and 300 nM were used. For all FcγRs, a dissociation of 120s was used, and the protein A surfaces were regenerated with a 30s pulse of 10 mM glycine pH 1.5 between injection cycles. The sensorgrams were double referenced and fit to the steady state model for affinity determination or a 1:1 binding model for kinetics and affinity if the dissociation phase was sufficiently slow for CD64a. Reported Ky values are the mean of two independent runs. All experiments were conducted at 25° C.

Binding to FcRn: Affinity of FcRn for the tested variants was measured by surface plasmon resonance (SPR) using the Biacore™ T200 System (Cytiva, Marlborough, MA) with PBS buffer containing 0.05% Tween 20 and 3.4 mM EDTA adjusted to pH 5.9. Neutravidin (ThermoFisher Scientific, Waltham, MA; Cat. 31000) at 10 ug/mL in 10 mM sodium acetate pH 4.5 was covalently immobilized on a CM5 sensor chip through standard amine coupling to 2000 RU (response units). Human FcRn with a C-terminal biotin on the large subunit at 5 ug/mL was injected at a flow rate of 20 uL/min for 20s to reach an FcRn capture level of 70 RUs. Each test variant was injected over the FcRn-immobilized surface using single cycle kinetics. Antibody variants were injected with increasing concentration between 5-1200 nM for 45s at 50 uL/min with a 180s dissociation using a single-cycle methodology in pH 5.9 running buffer. The FcRn surface was regenerated with a 30s pulse of PBST pH 7.4 between injection cycles. Sensorgrams were double-referenced and fit to a steady state binding model to generate affinity values. Reported K_{D}) values are the mean of two independent runs. All experiments were conducted at 25° C.

Results: The results are shown in Table 15.1. All cysteine insertion variants bound to FcRn with similar affinity, within 2-fold of the K_{D} for the wild-type control (v17427). The majority of the cysteine insertion variants also bound to all FcγRs with similar affinity, within 2-fold of the K_{D}) of the wild-type control (v17427), with the exception of v29001 (H_T299.5C) and v22765 (H_G237.5C) which showed significantly reduced binding to the FcγRs, and variant, v22768 (H_Q295.5C) which showed a reduction in binding to CD16aF, CD16aV, CD32aH, and CD32aR (>2-fold of the K_{D} of the wild-type control, v17427). The control cysteine insertion variant v29013 (H_S239.5C) also showed significantly reduced binding to the FcγRs as expected.

TABLE 15.1
SPR Binding Affinity (K_{D}) for Cysteine Insertion Variants to FcγRs and FcRn

| Variant | Construct | FcRn | CD64a | CD32aH | CD32aR | CD32bY | CD16aF | CD16aV |
|---|---|---|---|---|---|---|---|---|
| v17427 | wt control | 2.97E−07 | 4.86E−11 | 4.10E−07 | 5.88E−07 | 2.63E−06 | 1.45E−06 | 4.61E−07 |
| v32634 | wt control | 3.81E−07 | 7.32E−11 | 7.25E−07 | 1.11E−06 | 5.30E−06 | 1.97E−06 | 6.33E−07 |
| | (homoFc) | | | | | | | |
| v29001 | H_T299.5C | 2.08E−07 | 1.43E−07 | ND¹ | ND | ND | ND | ND |
| v22765 | H_G237.5C | 3.42E−07 | ND | ND | ND | ND | ND | ND |
| v22768 | H_Q295.5C | 2.95E−07 | 8.55E−11 | 1.36E−06 | 1.39E−06 | 4.72E−06 | 4.18E−06 | 1.35E−06 |
| v27321 | L_W148.5C | 2.79E−07 | 4.53E−11 | 4.27E−07 | 5.75E−07 | 2.51E−06 | 1.12E−06 | 4.14E−07 |
| v27322 | L_K149.5C | 2.23E−07 | 4.26E−11 | 3.97E−07 | 5.42E−07 | 2.43E−06 | 1.32E−06 | 4.46E−07 |
| v28983 | L_P40.5C | 2.24E−07 | 4.51E−11 | 4.31E−07 | 6.12E−07 | 2.71E−06 | 1.47E−06 | 4.88E−07 |
| v28989 | H_A9.5C | 3.32E−07 | 4.95E−11 | 4.16E−07 | 5.97E−07 | 2.67E−06 | 1.46E−06 | 4.53E−07 |
| v28993 | H_G169.5C | 3.20E−07 | 4.99E−11 | 4.33E−07 | 6.19E−07 | 2.67E−06 | 1.34E−06 | 4.20E−07 |
| v34685² | L_K39.5C | 3.22E−07 | 5.24E−11 | 4.55E−07 | 6.22E−07 | 2.81E−06 | 1.40E−06 | 4.54E−07 |
| v34686³ | L_K126.5C | 3.28E−07 | 4.40E−11 | 3.99E−07 | 5.21E−07 | 2.36E−06 | 1.25E−06 | 4.13E−07 |
| v29013 | H_S239.5C | 1.92E−07 | 8.61E−09 | ND | ND | ND | ND | ND |
| | (control) | | | | | | | |

¹ND = not determined
²Variant is the equivalent of v22760 in Table 2.2, but lacks the additional peptide ESSCDVKLV [SEQ ID NO: 2] fused to the C-terminal residue of the light chain.
³Variant is the equivalent of v22761 in Table 2.2, but the additional peptide ESSCDVKLV [SEQ ID NO: 2] fused to the C-terminal residue of the light chain.

### Example 16: Transferability of Cysteine Insertion Mutations

To demonstrate that the cysteine insertion mutations are transferable to other antibodies, four of the insertion sites were selected and introduced into four different antibodies as detailed in Table 16.1 for a total of seven new variants.

TABLE 16.1
Variants Generated to Demonstrate Transferability
of Cysteine Insertion Mutations

| | Cys Insertion | Variant | | |
|---|---|---|---|---|
| Domain | Site | # | Antibody | Target |
| Fc | H_T299.5 | v34014 | Trastuzumab | HER2 |
| | | v34217 | Anti-FRα | FRα |
| Fc | H_G237.5 | v34012 | Trastuzumab | HER2 |
| Fab | L_P40.5 | v33996 | CR8071¹ | Hemagglutinin (HA) |
| | | v34004 | H3² | HER3 |
| Fab | L_A9.5 | v34015 | SGNCD19a³ | CD19 |
| | | v34010 | Trastuzumab | HER2 |

¹Dreyfus, et al., 2012, Science, 337: 1343-1348
²U.S. Pat. No. 9,249,230
³U.S. Pat. No. 10,808,039

Cysteine insertion variants were prepared in the various antibody backgrounds following the same protocol as described in General Protocol 1. Variants based on trastuzumab, CR8071, H3 and SGNCD19a (v34014, v34012, v34010, v33996, v34004 and v34015) were conjugated to the drug-linker MTvcCompound 1 as described in General Procedure 2, with the following exception. For variants v34012 and v34010, once the reduction was complete, the reduced variants were buffer exchanged to PBS, pH 6.5 for oxidation either at RT or followed by conjugation with MTvcCompound 1. Samples prepared with oxidation at 4° C. showed better biophysical properties.

Variant v34217 was conjugated to 3 different camptothecin-based drug-linkers (MC-GGFG-Camptothecin 1, MC-GGFG-Camptothecin 2 and MC-GGFG-* Camptothecin 2) as described below. Drug-linker MC-GGFG-* Camptothecin 2 contains the same camptothecin analogue as drug-linker MC-GGFG-Camptothecin 2 but the linker is attached to a different position in the drug molecule.

A solution (647.2 uL) of variant v34217 (6 mg) was diluted to 6.4 mg/mL with a 5 mM solution of DTPA (diethylenetriamine pentaacetic acid, final concentration of 1 mM, vol: 188 uL) in PBS (pH 7.4), and to this solution was added 25 mM tris(2-carboxyethyl) phosphine (TCEP) (25 eq, 104 uL). Following incubation for 3h in a 37° C. water bath, reduced antibody was purified using 40 kDa 5 mL Zeba™ Spin Desalting Column (Thermo Fisher Scientific, Waltham, MA) pre-conditioned with 10 mM sodium acetate pH 5.5. The reduced antibody was subjected to overnight oxidation (18 hrs) with 25 molar excess dehydroascorbic acid (DHAA) (assuming 100% recovery from the Zeba™ column purification) at 4° C. to re-form the interchain disulphide bonds while keeping the inserted cysteine in reduced (free thiol) form. The reoxidized antibody was split into 3 even aliquots and conjugated to maleimide functionalized drug-linker MC-GGFG-Camptothecin 1, MC-GGFG-Camptothecin 2 or MC-GGFG-* Camptothecin 2 by incubating with 4 molar excesses of 10 mM DMSO stock of the drug-linker in the presence of 10% (v/v) DMSO at room temperature for 60-75 minutes after mixing thoroughly by pipetting. The conjugates formed were then purified by 40K Zeba™ columns pre-equilibrated with 10 mM sodium acetate pH 4.5.

Once the conjugation was complete, all ADCs were analysed by hydrophobic interaction chromatography (HIC), size-exclusion chromatography (SEC), liquid chromatography-mass spectrometry (LC-MS) and capillary electrophoresis-SDS (CE-SDS) as described in General Protocols 4, 5, 6 and 7, respectively.

The results are shown in Table 16.2 and demonstrate that, overall, the cysteine insertion mutations could be used successfully in different antibodies and to conjugate different drug-linkers. For most of the ADCs, DAR measured by either HIC or LC-MS were as expected (˜ DAR 2). For v33996, elution on HIC even as a naked antibody was relatively broad and hence, no distinct peak was observed after conjugation and neither DAR nor HIC-RRT could be measured. For v34012, a certain degree of non-specific conjugation was observed which led to a DAR of 2.1. The ADCs showed at least 95% monomer on SEC with exception of two ADCs: v34012 and v34010 conjugated to MTvcCompound 1. For these two ADCs, ˜15% LMWS observed after the oxidation (prior to addition of drug-linker), which remained unchanged after conjugation. Further optimization of oxidation step will improve the amount of LMWS observed for these two variants.

TABLE 16.2
Biophysical Properties

| | Insertion | | HIC | | UPLC-SEC | | | DAR |
|---|---|---|---|---|---|---|---|---|
| Variant | Position | Drug-Linker | DAR | RRT | Monomer % | HMWS % | LMWS % | (LC-MS) |
| v33996 | L_P40.5 | MTvcCompound 1 | ND¹ | ND¹ | 95 | 1 | 4 | 1.9 |
| v34004 | L_P40.5 | MTvcCompound 1 | 1.7 | 1.12 | 96 | 1 | 3 | 2.0 |
| v34015 | H_A9.5 | MTvcCompound 1 | 1.8 | 1.16 | 96 | 1 | 3 | 2.0 |
| v34014 | H_T299.5 | MTvcCompound 1 | 2.0 | 1.01 | 99 | 1 | 0 | 2.0 |
| v34012 | H_G237.5 | MTvcCompound 1 | 2.1 | 1.10 | 83 | 1 | 16 | 2.1³ |
| v34010 | H_A9.5 | MTvcCompound 1 | ND² | ND² | 84 | 3 | 13 | 1.9 |
| v34217 | H_T299.5 | MC-GGFG- | 2.0 | 1.18 | 100 | 0 | 0 | 2.0 |
| | | Camptothecin 1 | | | | | | |
| | | MC-GGFG- | 2.0 | 1.12 | 100 | 0 | 0 | 2.0 |
| | | Camptothecin 2 | | | | | | |
| | | MC-GGFG- | 2.0 | 1.12 | 100 | 0 | 0 | 2.0 |
| | | *Camptothecin 2 | | | | | | |

¹The naked variant appeared as a broader peak on HIC and thus DAR by HIC and HIC-RRT could not be calculated
²Due to multiple peaks, DAR by HIC and HIC-RRT could not be calculated
³Due to some degree of non-specific conjugation, DAR of >2 was observed

### Example 17: Preparation of Additional Dar-Tuned Antibody Drug Conjugates

In this example, multiple cysteine insertion sites were combined to generate antibodies capable of site-specific conjugation to provide DAR 4 and 6 ADCs. The combinations of cysteine insertion sites employed are shown in Table 17.1.

TABLE 17.1
Combinations of Cysteine Insertion Sites and Target DARs

| Target DAR | Cysteine Insertion Positions | Antibody | Variant |
|---|---|---|---|
| DAR 4 | H_A9.5 + L_P40.5 | Anti-cMet | v33943 |
| | H_G237.5 + L_P40.5 | Anti-cMet | v33948 |
| | H_A9.5 + H_G237.5 | Anti-cMet | v33952 |
| | H_T299.5 + L-P40.5 | Anti-cMet | v33955 |
| | H_A9.5 + H_T299.5 | Anti-cMet | v33959 |
| | H_G237.5 + H_T299.5 | Anti-cMet | v33961 |
| DAR 4 | H_T299.5, L_K126.5 | Anti-FRα | v34218 |
| | H_T299.5, L_P40.5 | Anti-FRα | v34456 |
| DAR 6 | H_G237.5 + H_T299.5 + L_P40.5 | Anti-cMet | v35073 |
| | H_A9.5 + H_T299.5 + L_P40.5 | Anti-cMet | v35074 |

Cysteine insertion variants were prepared following the protocol described in General Protocol 1. DAR 4 and DAR 6 anti-cMet variants were expressed in cysteine capped form with a L-cysteine cap, a glutathione cap, or a combination of both. Reduction, oxidation and conjugation to the drug-linker MTvcCompound 1 was carried out as described in General Procedure 2 with the following modifications. To account for additional cysteine capping, for DAR 4 and DAR 6 variants, reductions were performed with 30 and 40 eq. molar excess of tris(2-carboxyethyl) phosphine (TCEP), respectively, under similar conditions. Oxidation was performed with 30 and 40 eq. molar excess dehydroascorbic acid (DHAA) for DAR 4 and DAR 6 variants, respectively.

Conjugation of the DAR 4 anti-FRα variants to the drug-linkers MC-GGFG-Camptothecin 1, MC-GGFG-Camptothecin 2 and MC-GGFG-* Camptothecin 2, was carried out as described in Example 16.

Once the conjugation was complete, all ADCs were analysed by hydrophobic interaction chromatography (HIC), size-exclusion chromatography (SEC), liquid chromatography-mass spectrometry (LC-MS) and capillary electrophoresis-SDS (CE-SDS) as described in General Protocols 4, 5, 6 and 7, respectively.

The results are shown in Table 17.2 (anti-cMet antibody) and Table 17.3 (anti-FRα antibody).

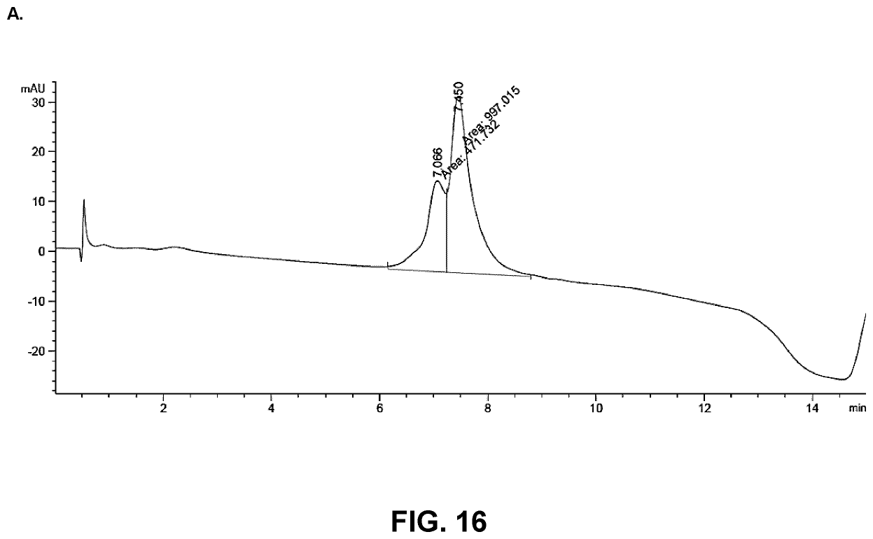

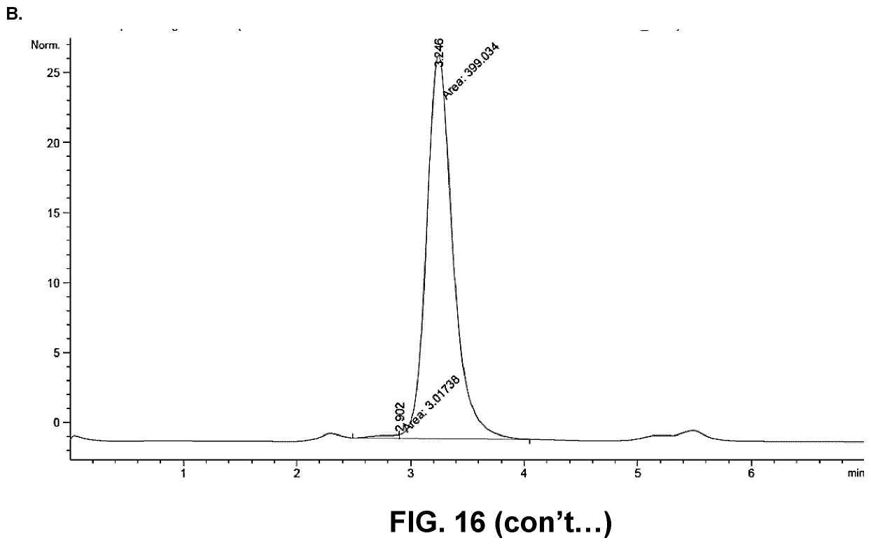

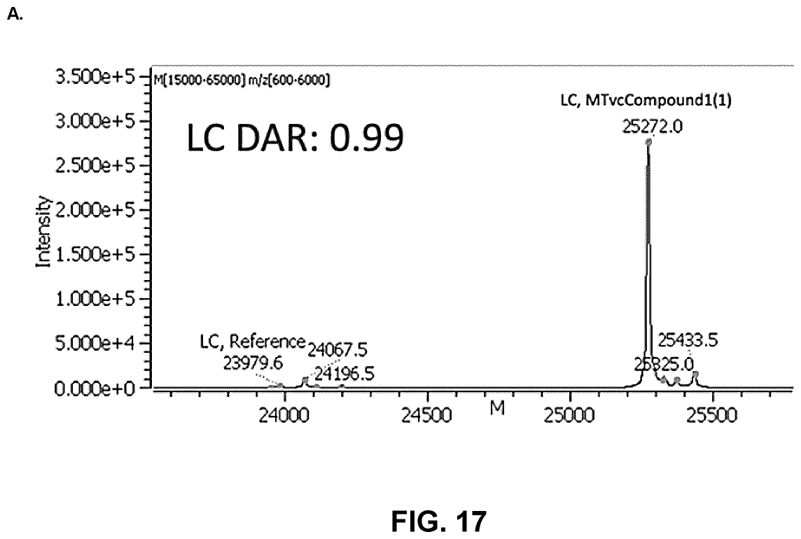

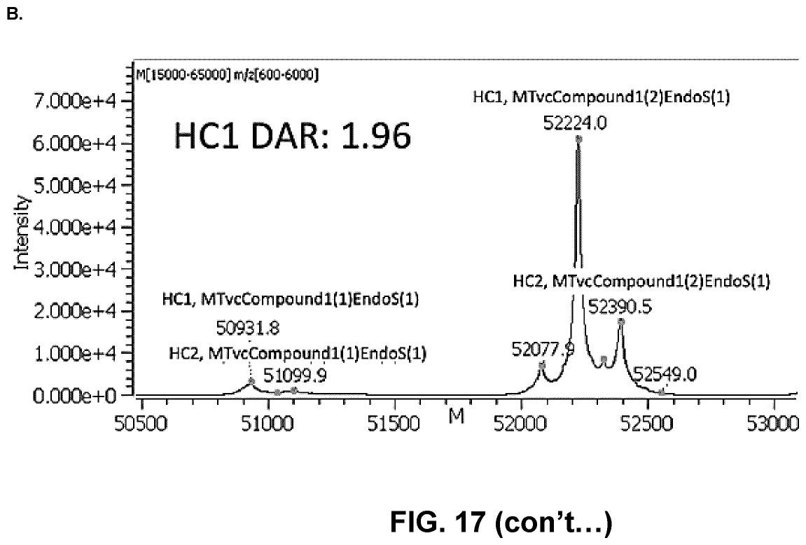

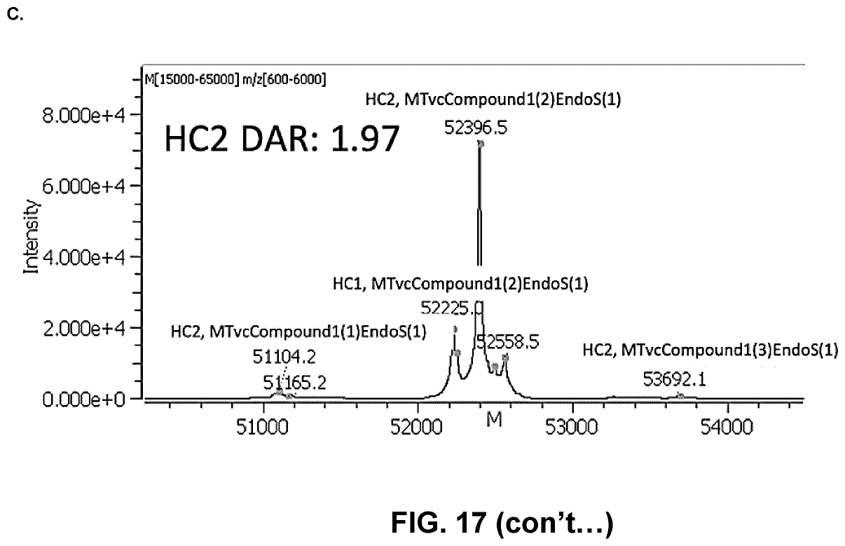

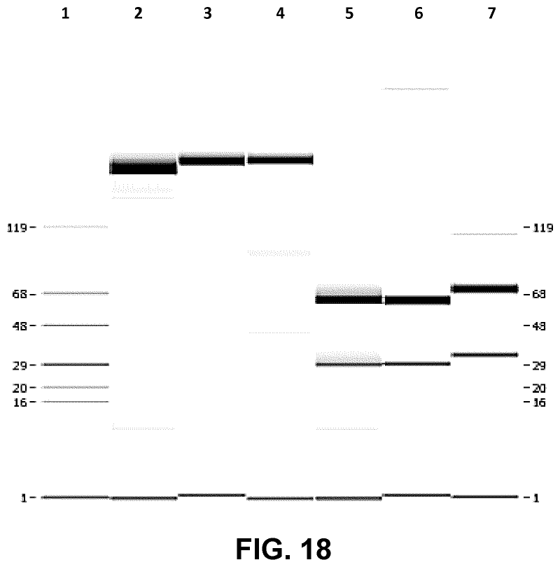

Overall, the site-specific DAR 4 and DAR 6 conjugations on the anti-cMet antibody backbone were successful with up to three different cysteine insertions being introduced into the antibody and conjugated to drug-linker. Representative HIC, SEC, LC-MS and CE-SDS profiles for the DAR 6 ADC, v35074-MTvcCompound 1 are shown in FIGS. 16-18.

For the ADCs v33943-MTvcCompound 1 and v35074-MTvcCompound 1, the DAR values calculated by HIC were less than expected due to the presence of co-eluting peaks. For the other anti-cMet ADCs, however, DAR calculated by HIC was close to the target DAR. DAR measured by LC-MS is more direct approach and should reflect closest to absolute values. With exception of v33952-MTvcCompound 1, all other anti-cMet ADCs showed at least 99% monomer on UPLC-SEC. Further optimization of the oxidation step for v33952 should reduce the amount of LMWS as this antibody showed >10% LMWS formation before the addition of drug-linker. CE-SDS run under non-denaturing conditions showed a higher proportion of half-antibodies compared to full size antibody for this variant.

For the site-specific DAR 4 conjugations on the anti-FRα antibody backbone, all ADCs appeared as >99% monomer on UPLC-SEC. By HIC, only one peak was observed for these ADCs. Both conjugated and unconjugated antibody had a very similar elution time, DAR estimates by HIC were less precise than LC-MS DAR calculations. Four out of six ADCs showed DAR 4 by LC-MS. Variant v34456 conjugated to either MC-GGFG-Camptothecin 1 or MC-GGFG-*Camptothecin 2 showed ˜25% non-specific conjugation, which can be reduced with further optimization.

TABLE 17.2
Biophysical Properties of Anti-cMet ADCs (DAR 4 and DAR 6)

| | | | | UPLC-SEC | | | |
|---|---|---|---|---|---|---|---|
| Target | | HIC | | Monomer | HMWS | LMWS | DAR |
| DAR | ADC | DAR | RRT | % | % | % | (LC-MS) |
| DAR 4 | v33943-MTvcCompound 1 | 3.2 | 1.28 | 100 | 0 | 0 | 4.0 |
| | v33948-MTvcCompound 1 | 4.0 | 1.21 | 100 | 0 | 0 | 3.9 |
| | v33952-MTvcCompound 1 | 3.9 | 1.26 | 88 | 1 | 11 | 4.0 |
| | v33955-MTvcCompound 1 | 4.0 | 1.11 | 100 | 0 | 0 | 4.0 |
| | v33959-MTvcCompound 1 | 4.0 | 1.13 | 99 | 1 | 0 | 4.0 |
| | v33961-MTvcCompound 1 | 4.0 | 1.08 | 99 | 1 | 0 | 3.8 |
| DAR 6 | v35073-MTvcCompound 1 | 6.0 | 1.16 | 99 | 1 | 1 | 5.9 |
| | v35074-MTvcCompound 1 | 5.4 | 1.31 | 99 | 1 | 0 | 5.9 |

TABLE 17.3
Biophysical Properties of Anti-FRα ADCs (DAR 4)

| Cysteine | | | | UPLC-SEC | | | |
|---|---|---|---|---|---|---|---|
| Insertion | | HIC | | Monomer | HMWS | LMWS | DAR |
| Positions | ADC | DAR^{#} | RRT | % | % | % | (LC-MS) |
| H_T299.5 | v34218-MC-GGFG- | 4.0 | 1.22 | 99 | 1 | 0 | 4.0 |
| L_126.5 | Camptothecin 1 | | | | | | |
| | v34218-MC-GGFG- | 4.0 | 1.11 | 99 | 1 | 0 | 4.0 |
| | Camptothecin 2 | | | | | | |
| | v34218-MC-GGFG- | 4.0 | 1.07 | 99 | 1 | 0 | 4.0 |
| | *Camptothecin 2 | | | | | | |
| H_T299.5 | v34456-MC-GGFG- | 4.0 | 1.17 | 100 | 0 | 0 | 4.4 |
| L_46.5 | Camptothecin 1 | | | | | | |
| | v34456-MC-GGFG- | 4.0 | 1.07 | 100 | 0 | 0 | 4.0 |
| | Camptothecin 2 | | | | | | |
| | v34456-MC-GGFG- | 4.0 | 1.06 | 99 | 1 | 0 | 4.3 |
| | *Camptothecin 2 | | | | | | |

^{#}Only a single HIC peak was observed

### Example 18: In Vitro Cytotoxicity of Dar-Tuned Anti-Frα ADCs

The cell growth inhibition (cytotoxicity) capabilities of the anti-FRα ADCs (DAR 2 and DAR 4) generated in Examples 16 and 17 were compared to stochastic DAR 4 ADCs in a 3D cytotoxicity assay as described below using the FRα-expressing cancer cell lines JEG-3 (placental choriocarcinoma) and T-47D (breast carcinoma).

Briefly, 3,000 cells/well were seeded in 384-well Ultra-Low Attachment (ULA) plates, centrifuged at 200×g for 2 minutes, and incubated under standard culturing conditions for 3 days to allow spheroid formation (1 spheroid/well). After 3 days, spheroids were treated with a titration of test article prepared in complete growth medium and incubated under standard culturing conditions for 6 days. After incubation, CellTiter-Glo® 3D reagent (Promega Corporation, Madison, WI) was spiked in all wells. Plates were incubated in the dark at room temperature for 1 hour and luminescence was quantified using a BioTek Cytation 5 Cell Imaging Multi-Mode Reader (Agilent Technologies, Inc., Santa Clara, CA). Based on blank wells (no test article added), percent cytotoxicity values were calculated and plotted against test article concentration using GraphPad Prism 9 software (GraphPad Software, San Diego, CA).

The results are shown in Table 18.1. Site-specific DAR 4 ADCs exhibited comparable 3D in vitro cytotoxicity to payload-matched stochastic DAR 4 ADCs against both JEG-3 and T-47D spheroids. Site-specific DAR 2 ADCs exhibited log-fold lower potency than payload-matched DAR 4 ADCs against JEG-3 and T-47D spheroids, as expected due to the lower drug loading on a DAR 2 ADC.

DAR 4 site-specific and stochastic ADCs including Camptothecin 2 showed similar potency to payload-matched DAR 8 stochastic ADC against both JEG-3 and T-47D spheroids.

DAR 4 site-specific and stochastic ADCs including Camptothecin 1 showed similar potency to payload-matched DAR 8 stochastic ADC against high FRα-expressing JEG-3 spheroids. In the lower FRα-expressing T-47D spheroids, the DAR 4 ADCs showed a drug-loading dependent dose response, with the payload-matched DAR 8 stochastic ADC showing 3-9-fold higher potency.

TABLE 18.1
In vitro Cytotoxicity of DAR-Tuned ADCs

| | | | 3D EC50 (nM) | |
|---|---|---|---|---|
| | | Insertion | JEG-3 | T-47D |
| Test Sample | DAR | Position | Spheroid | Spheroid |
| v30384-MT-GGFG-Camptothecin 1 | 8.0 | stochastic | 0.45 | 2.24 |
| v30384-MT-GGFG-Camptothecin 2 | 8.0 | stochastic | 0.71 | 0.73 |
| v36675-MT-GGFG-Camptothecin 1 | 3.5 | stochastic | 0.78 | 19.75 |
| v36675-MT-GGFG-Camptothecin 2 | 4.2 | stochastic | 0.66 | 0.80 |
| v34217-MC-GGFG-Camptothecin 1 | 2.0 | H_T299.5 | 9.40 | 15.57 |
| v34217-MC-GGFG-Camptothecin 2 | 2.0 | | 12.31 | 15.75 |
| v34218-MC-GGFG-Camptothecin 1 | 4.0 | H_T299.5 + | 0.79 | 9.51 |
| v34218-MC-GGFG-Camptothecin 2^{#} | 4.0 | L_K126.5 | 1.06 | 0.82 |
| v34456-MC-GGFG-Camptothecin 1^{#} | 4.4 | H_T299.5 + | 0.68 | 7.37 |
| v34456-MC-GGFG-Camptothecin 2^{#} | 4.0 | L_P40.5 | 1.02 | 0.57 |
| DXd1 (free payload) | — | — | 1.09 | 1.54 |
| Camptothecin 1 (free payload) | — | — | 1.80 | 1.56 |
| Camptothecin 2 (free payload) | — | — | 2.12 | 0.64 |

^{#}ADCs had more than 1 mol %/DAR free-toxin as measured by RP-LCMS

The disclosures of all patents, patent applications, publications and database entries referenced in this specification are hereby specifically incorporated by reference in their entirety to the same extent as if each such individual patent, patent application, publication and database entry were specifically and individually indicated to be incorporated by reference.

Modifications of the specific embodiments described herein that would be apparent to those skilled in art are intended to be included within the scope of the following claims.

1. A cysteine engineered antibody construct
comprising one or more cysteine insertion mutations selected from:
(a) an insertion of a cysteine residue between positions 39 and 40 in a VL domain;
(b) an insertion of a cysteine residue between positions 40 and 41 in a VL domain;
(c) an insertion of a cysteine residue between positions 126 and 127 in a CL domain;
(d) an insertion of a cysteine residue between positions 148 and 149 in a CL domain;
(e) an insertion of a cysteine residue between positions 149 and 150 in a CL domain;
(f) an insertion of a cysteine residue between positions 9 and 10 in a VH domain;
(g) an insertion of a cysteine residue between positions 169 and 170 in a CHI domain;
(h) an insertion of a cysteine residue between positions 237 and 238 in a CH2 domain;
(i) an insertion of a cysteine residue between positions 295 and 296 in a CH2 domain, and
(j) an insertion of a cysteine residue between positions 299 and 300 in a CH2 domain,
wherein the numbering of amino acids in the VL, CL, VH and CH1 domains is Kabat numbering and the numbering of amino acids in the CH2 domain is EU numbering, and
wherein antibody construct is based on an immunoglobulin G (IgG).

2. The cysteine engineered antibody construct according to claim 1, wherein the melting temperature (Tm) of the domain of the cysteine engineered antibody construct comprising the cysteine insertion mutation is within 0° C. to 8° C. of the Tm of the same domain in the corresponding parental antibody construct that lacks the cysteine insertion mutation.

3. The cysteine engineered antibody construct according to claim 1, wherein antibody construct comprises one or three cysteine insertion mutations.

4. The cysteine engineered antibody construct according to claim 1 or 2, wherein antibody construct comprises two cysteine insertion mutations.

5. The cysteine engineered antibody construct according to claim 4, wherein the two cysteine insertion mutations are symmetrical mutations.

6. (canceled)

7. The cysteine engineered antibody construct according to claim 3, wherein antibody construct comprises three cysteine insertion mutations, and wherein two of the three cysteine insertion mutations are symmetrical mutations.

8. The cysteine engineered antibody construct according to claim 3, wherein each cysteine insertion mutation is independently selected from:
(a) an insertion of a cysteine residue between positions 9 and 10 in a VH domain;
(b) an insertion of a cysteine residue between positions 40 and 41 in a VL domain;
(c) an insertion of a cysteine residue between positions 237 and 238 in a CH2 domain, and
(d) an insertion of a cysteine residue between positions 299 and 300 in a CH2 domain.

9. The cysteine engineered antibody construct according to claim 3, wherein antibody construct comprises one cysteine insertion mutation, and wherein the cysteine insertion mutation is selected from:
(a) an insertion of a cysteine residue between positions 9 and 10 in a VH domain;
(b) an insertion of a cysteine residue between positions 237 and 238 in a CH2 domain, and
(c) an insertion of a cysteine residue between positions 299 and 300 in a CH2 domain.

10. The cysteine engineered antibody construct according to claim 1, wherein antibody construct comprises four or six cysteine insertion mutations.

11. The cysteine engineered antibody construct according to claim 10, wherein the cysteine insertion mutations are selected from:
(a) an insertion of a cysteine residue between positions 40 and 41 in a VL domain;
(b) an insertion of a cysteine residue between positions 126 and 127 in a CL domain;
(c) an insertion of a cysteine residue between positions 9 and 10 in a VH domain;
(d) an insertion of a cysteine residue between positions 237 and 238 in a CH2 domain, and
(e) an insertion of a cysteine residue between positions 299 and 300 in a CH2 domain.

12. (canceled)

13. The cysteine engineered antibody construct according to claim 1, wherein antibody construct comprises one or more antigen-binding domains, at least one of the antigen-binding domains comprising a VH domain or a VH domain and a VL domain.

14. The cysteine engineered antibody construct according to claim 13, wherein the antibody construct comprises two antigen-binding domains.

15. The cysteine engineered antibody construct according to claim 14, wherein the antibody construct is bispecific.

16. The cysteine engineered antibody construct according to claim 13, wherein at least one of the antigen-binding domains binds to a tumor-associated antigen.

17. The cysteine engineered antibody construct according to claim 1, wherein antibody construct comprises an Fc region.

18. The cysteine engineered antibody construct according to claim 1, wherein antibody construct is based on an IgG1.

19. The cysteine engineered antibody construct according to claim 1, wherein the IgG is a human IgG.

20. The cysteine engineered antibody construct according to claim 1, wherein antibody construct comprises a heterodimeric Fc region.

21. The cysteine engineered antibody construct according to claim 20, wherein the heterodimeric Fc region comprises a modified CH3 domain comprising amino acid mutations that promote formation of the heterodimeric Fc over a homodimeric Fc.

22. A conjugate comprising the cysteine engineered antibody construct according to claim 1, and one or more active agents conjugated to each of the one or more inserted cysteine residues.

23. A conjugate having Formula (I): 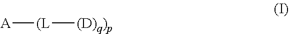
wherein:
A is a cysteine engineered antibody construct;
L is a linker;
D is an active agent;
q is an integer between 1 and 4, and
p is an integer between 1 and 8,
wherein the cysteine engineered antibody construct comprises one or more cysteine insertion mutations selected from:
(a) an insertion of a cysteine residue between positions 39 and 40 in a VL domain;
(b) an insertion of a cysteine residue between positions 40 and 41 in a VL domain;
(c) an insertion of a cysteine residue between positions 126 and 127 in a CL domain;
(d) an insertion of a cysteine residue between positions 148 and 149 in a CL domain;
(e) an insertion of a cysteine residue between positions 149 and 150 in a CL domain;
(f) an insertion of a cysteine residue between positions 9 and 10 in a VH domain;
(g) an insertion of a cysteine residue between positions 169 and 170 in a CHI domain;
(h) an insertion of a cysteine residue between positions 237 and 238 in a CH2 domain;
(i) an insertion of a cysteine residue between positions 295 and 296 in a CH2 domain, and
(j) an insertion of a cysteine residue between positions 299 and 300 in a CH2 domain,
wherein the numbering of amino acids in the VL, CL, VH and CH1 domains is Kabat numbering and the numbering of amino acids in the CH2 domain is EU numbering,
wherein the cysteine engineered antibody construct is based on an immunoglobulin G (IgG), and
wherein each D is linked to an inserted cysteine residue via L.

24. The conjugate according to claim 23, wherein the conjugate has Formula (II): 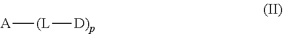
wherein:
A is the cysteine engineered antibody construct;
L is the linker;
D is the active agent, and
p is an integer between 1 and 8.

25. The conjugate according to claim 23, wherein active agent is a diagnostic agent or a labelling agent.

26. The conjugate according to claim 23, wherein active agent is a therapeutic agent.

27. A composition comprising the conjugate according to claim 23, and a pharmaceutically acceptable carrier or diluent.

28. A method of treating a disease or disorder in a subject in need thereof comprising administering an effective amount of the conjugate according to claim 26.

29.-30. (canceled)

31. A method of preparing the conjugate according to claim 23 comprising submitting the cysteine engineered antibody construct to reducing conditions such that the thiol group of the one or more inserted cysteine residues is reduced, and reacting a thiol reactive linker-active agent with the antibody construct under conditions that permit formation of a bond between the linker and the reduced thiol.

32. A method of preparing an antibody-drug conjugate having a pre-determined drug-to-antibody ratio (DAR), the method comprising:
(i) providing a cysteine engineered antibody construct comprising one or more cysteine insertion mutations selected from:
(a) an insertion of a cysteine residue between positions 39 and 40 in a VL domain;
(b) an insertion of a cysteine residue between positions 40 and 41 in a VL domain;
(c) an insertion of a cysteine residue between positions 126 and 127 in a CL domain;
(d) an insertion of a cysteine residue between positions 148 and 149 in a CL domain;
(e) an insertion of a cysteine residue between positions 149 and 150 in a CL domain;
(f) an insertion of a cysteine residue between positions 9 and 10 in a VH domain;
(g) an insertion of a cysteine residue between positions 169 and 170 in a CHI domain;
(h) an insertion of a cysteine residue between positions 237 and 238 in a CH2 domain;
(i) an insertion of a cysteine residue between positions 295 and 296 in a CH2 domain, and
(j) an insertion of a cysteine residue between positions 299 and 300 in a CH2 domain, and
(ii) reacting the cysteine engineered antibody construct with a drug-linker to provide the antibody-drug conjugate;
wherein the pre-determined DAR is 1, 2, 3, 4, 5, 6, 7 or 8, and the cysteine engineered antibody construct comprises the same number of cysteine insertion mutations as the pre-determined DAR,
wherein the numbering of amino acids in the VL, CL, VH and CH1 domains is Kabat numbering and the numbering of amino acids in the CH2 domain is EU numbering, and
wherein the cysteine engineered antibody construct is based on an immunoglobulin G (IgG).

33. The method according to claim 32, wherein the pre-determined DAR is 2.

34. The method according to claim 32, wherein;
(i) the pre-determined DAR is 1 or 3, and the cysteine insertion mutations are selected from:
(a) an insertion of a cysteine residue between positions 9 and 10 in a VH domain;
(b) an insertion of a cysteine residue between positions 40 and 41 in a VL domain;
(c) an insertion of a cysteine residue between positions 237 and 238 in a CH2 domain, and
(d) an insertion of a cysteine residue between positions 299 and 300 in a CH2 domain, or
(ii) the pre-determined DAR is 4 or 6, and the cysteine insertion mutations are selected from:
(a) an insertion of a cysteine residue between positions 40 and 41 in a VL domain;
(b) an insertion of a cysteine residue between positions 126 and 127 in a CL domain;
(c) an insertion of a cysteine residue between positions 9 and 10 in a VH domain;
(d) an insertion of a cysteine residue between positions 237 and 238 in a CH2 domain, and
(e) an insertion of a cysteine residue between positions 299 and 300 in a CH2 domain.

35.-37. (canceled)

38. A polynucleotide or set of polynucleotides encoding the cysteine engineered antibody construct according to claim 1.

39. A vector comprising one or more polynucleotides encoding the cysteine engineered antibody construct according to claim 1.

40. A host cell comprising the vector according to claim 39.
